(19) United States
(12) Patent Application Publication
(10) Pub. No.: US 2011/0171569 A1
(43) Pub. Date: Jul. 14, 2011

(54) SULFONIUM DERIVATIVES AND THE USE THEROF AS LATENT ACIDS

(76) Inventors: Yuichi Nishimae, Osaka (JP); Toshikage Asakura, Osaka (JP); Hitoshi Yamato, Hyogo (JP)

(21) Appl. No.: 12/996,795

(22) PCT Filed: Jun. 2, 2009
(86) PCT No.: PCT/EP2009/056703

§ 371 Date: Jan. 28, 2011

(30) Foreign Application Priority Data
Jun. 12, 2008 (EP) 08158090.4

(51) Int. Cl. G03F 7/004; C07D 335/08; C07D 339/08; C07D 413/02; C07D 285/16; C07D 327/08; C08F 228/06; G03F 7/20; G03F 1/00

(52) U.S. Cl. 430/7; 549/27; 549/17; 544/145; 544/5; 549/16; 526/256; 526/257; 430/286.1; 430/325

(57) ABSTRACT

Compounds of the formula (I), wherein R₁, R₂ and R₃ for example are hydrogen, halogen, CN, C₁-C₁₈alkyl, C₁-C₁₀ haloalkyl, (CO)R₈, (CO)OR₄, or (CO)NR₅R₆; Y is O, S or CO; D₂, D₃ and D₄ for example are a direct bond, O, S, NR₇, CO, O(CO), (CO)O, S(CO), (CO)S, NR₇(CO), (CO)NR₇, SO, SO₂, or OSO₂, C₁-C₁₈alkylene, C₃-C₃₀cycloalkylene, C₂-C₁₂alkenylene, C₄-C₃₀cycloalkenylene, Ar₁; Ar₁, Ar₂ and Ar₃ are for example phenylene, R₄, R₅, R₆, R₇ and R₈ are for example hydrogen, C₃-C₃₀-cycloalkyl, C₁-C₁₈alkyl, C₁-C₁₀haloalkyl, C₂-C₁₂alkenyl, C₄-C₃₀cycloalkenyl, phenyl-C₁C₃-alkyl; X⁻ is Formulae (IA), (IB) or (IC); R₁₀ is for example C₁-C₁₈alkyl, C₁-C₁₀haloalkyl, camphoryl, phenyl-C₁-C₃alkyl, C₃-C₃₀cycloalkyl; and R₁₁, R₁₂, R₁₃, R₁₄ and R₁₅ are for example C₁-C₁₀haloalkyl; are useful as polymerizable photolatent acids.

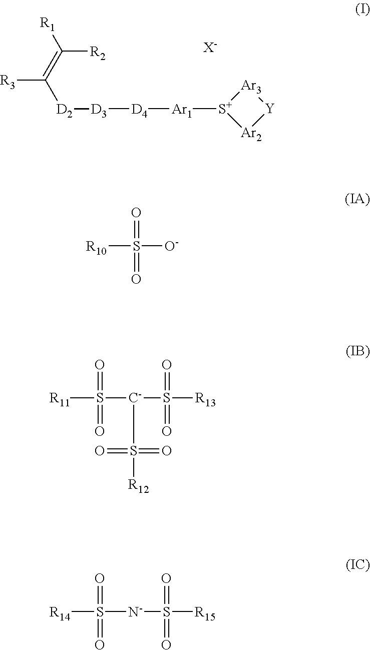

The invention relates to new sulfonium salts bearing polymerizable ethylenically unsaturated groups, polymers comprising repeating units derived from the said compounds, chemically amplified photoresist compositions comprising said compounds and/or said polymers and to the use of the compounds and/or polymers as latent acids, which can be activated by irradiation with actinic electromagnetic radiation and electron beams.

In EP473547 onium salt bearing an unsaturated double bond, e.g., 4-acryloyloxyphenyl-diphenylsulfonium salt, and a polymer comprising said onium salt are described. In WO2002-73308 and proceedings of SPIE (2001), 4345, 521-527 a polymerizable sulfonium salt compound, 4-methacryloyloxyphenyldimethylsulfonium salt, and polymers comprising said sulfonium salt are described as chemically amplified resist. In JP2005-84365 4-(meth)acryloyloxyphenyldiphenylsulfonium salt and resists containing a polymer comprising said sulfonium salt are disclosed. In JP2006-259508 and JP2006-259509 chemically amplified resist composed of photolatent acid and polymer comprising a triarylsulfonium salt repeating unit, e.g., 10-[4-(methacryloyloxy)phenyl]-9H-thioxanthenium, 5-(4-methacryloyloxyphenyl)dibenzothiophenium, and 5-[4-(4-vinylbenzyloxy)phenyl]dibenzothiophenium salts, is disclosed.

In the art exists a need for reactive latent acid donors that are thermally and chemically stable and that, after being activated by light, UV-radiation, X-ray irradiation or electron beams can be used as catalysts for a variety of acid-catalysed reactions, such as polycondensation reactions, acid-catalysed depolymerization reactions, acid-catalysed electrophilic substitution reactions or the acid-catalysed removal of protecting groups. A particular need exists for latent acid catalysts with high stability, high sensitivity and high resolution not only in the Deep-UV range but also in a wide range of wavelengths such as for example g-line (436 nm), i-line (365 nm), KrF (248 nm), ArF (193 nm) and EUV (13.5 nm; extreme-ultra-violet). In addition, a new need emerges for latent acid catalysts with non-outgassing properties even after the decomposition by exposure, especially for EUV and EB (electron beam) lithography, wherein the exposure is carried out under vacuum conditions.

Surprisingly, it has now been found that specific sulfonium salts and polymers attaching sulfonium salts via the chromophore moiety, as described below, are stable and highly active against the wide range of light sources. The sulfonium salts and polymers attaching sulfonium salts via the chromophore moiety in the present invention are especially suitable as catalysts for the aforementioned acid catalyzed reactions in chemically amplified photoresist applications. In addition, the sulfonium salts and polymers with sulfonium salts attached via the chromophore moiety in the present invention are suitable for EUV and EB lithography due to their non-outgassing properties. Furthermore, chemically amplified photoresist compositions comprising sulfonium salts and polymers with sulfonium salts attached via the chromophore moiety of the present invention provide a high photospeed and high resolution.

Subject of the invention is a compound of the formula I

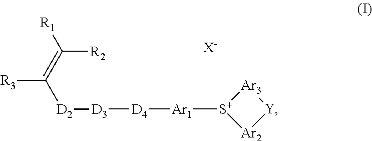

wherein
R₁, R₂ and R₃ independently of each other are hydrogen, halogen, CN, C₁-C₁₈alkyl, C₁-C₁₀haloalkyl, (CO)R₈, (CO)OR₄, or (CO)NR₅R₆; Y is O, S or CO;
D₂, D₃ and D₄ independently of each other are a direct bond, O, S, NR₇, CO, O(CO), (CO)O, S(CO), (CO)S, NR₇(CO), (CO)NR₇, SO, SO₂, or OSO₂, C₁-C₁₈alkylene, C₃-C₃₀cycloalkylene, C₂-C₁₂alkenylene, C₄-C₃₀cycloalkenylene, Ar₁;
or independently of each other are C₂-C₁₈alkylene which is interrupted by one or more O, S, NR₇, O(CO), (CO)O, (CO)NR₇ or NR₇(CO);
or independently of each other are C₃-C₃₀cycloalkylene which is interrupted by one or more O, S, NR₇, O(CO) (CO)O, (CO)NR₇, or NR₇(CO);
or independently of each other are C₄-C₃₀cycloalkenylene which is interrupted by one or more O, S, NR₇, O(CO), (CO)O, (CO)NR₇ or NR₇(CO);
wherein D₂, D₃ and D₄ as C₁-C₁₈alkylene, C₃-C₃₀cycloalkylene, C₂-C₁₂alkenylene, C₄-C₃₀cycloalkenylene, Ar₁, interrupted C₂-C₁₈alkylene, interrupted C₃-C₃₀cycloalkylene and interrupted C₄-C₃₀cycloalkenylene optionally are substituted by one or more Ar, OH, C₁-C₁₈alkyl, C₁-C₁₀haloalkyl, phenyl-C₁-C₃-alkyl, C₃-C₃₀cycloalkyl, halogen, NO₂, CN, C₁-C₁₈alkoxy, phenoxy, phenoxycarbonyl, phenylthio, phenylthiocarbonyl, NR₅R₆, C₁-C₁₂alkylthio, C₂-C₁₈alkoxycarbonyl, C₂-C₁₀haloalkanoyl, halobenzoyl, C₁-C₁₈alkylsulfonyl, phenylsulfonyl, (4-methylphenyl)sulfonyl, C₁-C₁₈alkylsulfonyloxy, phenylsulfonyloxy, (4-methylphenyl)sulfonyloxy, C₂-C₁₈alkanoyl, C₂-C₁₈alkanoyloxy, benzoyl and/or by benzoyloxy;
or R₂ and D₂, together with the ethylenically unsaturated double bond to which they are attached, form a 5-, 6- or 7-membered ring which optionally is interrupted by one or more O, S, NR₇ or CO;
or R₃ and D₂ together with the carbon of the ethylenically unsaturated double bond to which they are attached form C₃-C₃₀cycloalkyl which optionally is interrupted by one or more O, S, NR₇ or CO;
Ar₁ is phenylene, biphenylene, naphthylene,

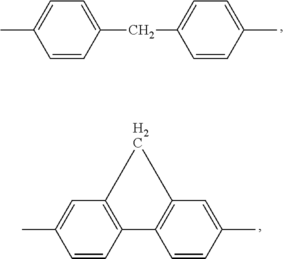

heteroarylene, oxydiphenylene or

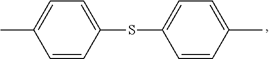

all of which are optionally substituted by one or more C₃-C₃₀cycloalkyl, C₁-C₁₈alkyl, C₁-C₁₀haloalkyl, C₂-C₁₂alkenyl, C₄-C₃₀cycloalkenyl, phenyl-C₁-C₃-alkyl,
or are substituted by C₂-C₁₈alkyl which is interrupted by one or more O, S, NR₇, O(CO), (CO)O, (CO)NR₇ and/or NR₇(CO);
or are substituted by C₃-C₃₀cycloalkyl which is interrupted by one or more O, S, NR₇, O(CO), (CO)O, (CO)NR₇ and/or NR₇(CO);
or are substituted by C₄-C₃₀cycloalkenyl which is interrupted by one or more O, S, NR₇, O(CO), (CO)O, (CO)NR₇ and/or NR₇(CO);
or are substituted by halogen, NO₂, CN, Ar, (CO)R₈, (CO)OR₄, (CO)NR₅R₆, O(CO)R₈, O(CO)OR₄, O(CO)NR₅R₆, NR₇(CO)R₈, NR₇(CO)OR₄, OR₄, NR₅R₆, SR₇, SOR₈, SO₂R₈ and/or —OSO₂R₈,
wherein optionally the substituents C₁-C₁₈alkyl, C₂-C₁₂alkenyl, (CO)R₈, (CO)OR₄, (CO)NR₅R₆, O(CO)R₈, O(CO)OR₄, O(CO)NR₅R₆, NR₇(CO)R₈, NR₇(CO)OR₄, OR₄, NR₅R₆, SR₇, SOR₈, SO₂R₈ and/or OSO₂R₈ form 5-, 6- or 7-membered rings, via the radicals C₁-C₁₈alkyl, C₂-C₁₂alkenyl, R₄, R₅, R₆, R₇ and/or R₈, with further substituents on the phenylene, biphenylene, naphthylene,

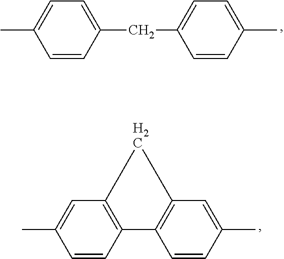

heteroarylene, oxydiphenylene or

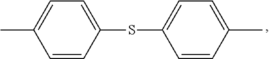

or with one of the carbon atoms of the phenylene, biphenylene, naphthylene,

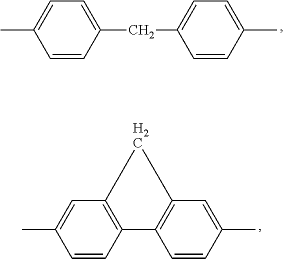

heteroarylene, oxydiphenylene or

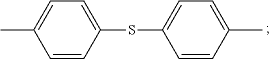

wherein all Ar₁ optionally additionally are substituted by a group having a —O—C-bond or a —O—Si-bond which cleaves upon the action of an acid;
Ar₂ and Ar₃ independently of each other are phenylene or naphthylene,
wherein the phenylene or naphthylene are optionally substituted by one or more C₃-C₃₀cycloalkyl, C₁-C₁₈alkyl, C₁-C₁₀haloalkyl, C₂-C₁₂alkenyl, C₄-C₃₀cycloalkenyl, phenyl-C₁-C₃-alkyl;
or by C₂-C₁₈alkyl which is interrupted by one or more O, S, NR₇, O(CO), (CO)O, (CO)NR₇, and/or NR₇(CO);
or by C₃-C₃₀cycloalkyl which is interrupted by one or more O, S, NR₇, O(CO), (CO)O, (CO)NR₇, and/or NR₇(CO);
or by C₄-C₃₀cycloalkenyl which is interrupted by one or more O, S, NR₇, O(CO), (CO)O, (CO)NR₇, and/or NR₇(CO);
or are substituted by halogen, NO₂, CN, Ar, (CO)R₈, (CO)OR₄, (CO)NR₅R₆, O(CO)R₈, O(CO)OR₄, O(CO)NR₅R₆, NR₇(CO)R₈, NR₇(CO)OR₄, OR₄, NR₅R₆, SR₇, SOR₈, SO₂R₈ and/or OSO₂R₈,
optionally the substituents C₁-C₁₈alkyl, C₂-C₁₂alkenyl, (CO)R₈, (CO)OR₄, (CO)NR₅R₆, O(CO)R₈, O(CO)OR₄, O(CO)NR₅R₆, NR₇(CO)R₈, NR₇(CO)OR₄, OR₄, NR₅R₆, SR₇, SOR₈, SO₂R₈ and/or OSO₂R₈ form 5-, 6- or 7-membered rings, via the radicals C₁-C₁₈alkyl, C₂-C₁₂alkenyl, R₄, R₅, R₆, R₇ and/or R₈, with further substituents on the phenylene or naphthylene or with one of the carbon atoms of the phenylene or naphthylene;
wherein all Ar₂ and Ar₃ optionally additionally are substituted by a group having a —O—C-bond or a —O—Si-bond which cleaves upon the action of an acid;
R₄ is hydrogen, C₃-C₃₀cycloalkyl, C₁-C₁₈alkyl, C₁-C₁₀haloalkyl, C₂-C₁₂alkenyl, C₄-C₃₀cycloalkenyl, phenyl-C₁-C₃-alkyl;
or is C₂-C₁₈alkyl which is interrupted by one or more O, S, NR₇, O(CO), (CO)O, (CO)NR₇, and/or NR₇(CO);
or is C₃-C₃₀cycloalkyl which is interrupted by one or more O, S, NR₇, O(CO), (CO)O, (CO)NR₇ and/or NR₇(CO);
or is C₄-C₃₀cycloalkenyl which is interrupted by one or more O, S, NR₇, O(CO), (CO)O, CO(CO)NR₇ and/or NR₇(CO);
or R₄ is Ar, (CO)R₈, (CO)OR₈, (CO)NR₅R₆, and/or SO₂R₈;
wherein R₄ as C₃-C₃₀cycloalkyl, C₁-C₁₈alkyl, C₁-C₁₀haloalkyl, C₂-C₁₂alkenyl, C₄-C₃₀cycloalkenyl, phenyl-C₁-C₃-alkyl, interrupted C₂-C₁₈alkyl, interrupted C₃-C₃₀cycloalkyl, interrupted C₄-C₃₀cycloalkenyl and Ar optionally is substituted by one or more Ar, OH, C₁-C₁₈alkyl, C₁-C₁₀haloalkyl, phenyl-C₁-C₃-alkyl, C₃-C₃₀cycloalkyl, halogen, NO₂, CN, C₁-C₁₈alkoxy, phenoxy, phenoxycarbonyl, phenylthio, phenylthiocarbonyl, NR₅R₆, C₁-C₁₂alkylthio, C₂-C₁₈alkoxycarbonyl, C₂-C₁₀haloalkanoyl, halobenzoyl, C₁-C₁₈alkylsulfonyl, phenylsulfonyl, (4-methylphenyl)sulfonyl, C₁-C₁₈alkylsulfonyloxy, phenylsulfonyloxy, (4-methylphenyl)sulfonyloxy, C₂-C₁₈alkanoyl, C₂-C₁₈alkanoyloxy, benzoyl and/or by benzoyloxy;
R₅ and R₆ independently of each other are hydrogen, C₃-C₃₀cycloalkyl, C₁-C₁₈alkyl, C₁-C₁₀haloalkyl, C₂-C₁₂alkenyl, C₄-C₃₀cycloalkenyl, phenyl-C₁-C₃-alkyl;
or independently of each other are C₂-C₁₈alkyl which is interrupted by one or more O, S, NR₇, O(CO), (CO)O, C(CO)NR₇, and/or NR₇(CO);
or independently of each other are C₃-C₃₀cycloalkyl which is interrupted by one or more O, S, NR₇, O(CO), (CO)O, (CO)NR₇, and/or NR₇(CO);
or independently of each other are C₄-C₃₀cycloalkenyl which is interrupted by one or more O, S, NR₇, O(CO), (CO)O, (CO)NR₇, and/or NR₇(CO);
or R₅ and R₆ independently of each other are Ar, (CO)R₈, (CO)OR₄ and/or —SO₂R₈;
wherein R₅ and R₆ as C₃-C₃₀cycloalkyl, C₁-C₁₈alkyl, C₁-C₁₀haloalkyl, C₂-C₁₂alkenyl, C₄-C₃₀cycloalkenyl, phenyl-C₁-C₃-alkyl, interrupted C₂-C₁₈alkyl, interrupted C₃-C₃₀cycloalkyl, interrupted C₄-C₃₀cycloalkenyl and Ar optionally are substituted by one or more Ar, OH, C₁-C₁₈alkyl, C₁-C₁₀haloalkyl, phenyl-C₁-C₃-alkyl, C₃-C₃₀cycloalkyl, halogen, NO₂, CN, C₁-C₁₈alkoxy, phenoxy, phenoxycarbonyl, phenylthio, phenylthiocarbonyl, C₁-C₁₈dialkylamino,
C₁-C₁₂alkylthio, C₂-C₁₈alkoxycarbonyl, C₂-C₁₀haloalkanoyl, halobenzoyl, C₁-C₁₈alkylsulfonyl, phenylsulfonyl, (4-methylphenyl)sulfonyl, C₁-C₁₈alkylsulfonyloxy, phenylsulfonyloxy, (4-methylphenyl)sulfonyloxy, C₂-C₁₈alkanoyl, C₂-C₁₈alkanoyloxy, benzoyl and/or by benzoyloxy;
or R₅ and R₆, together with the nitrogen atom to which they are attached, form a 5-, 6- or 7-membered ring which optionally is interrupted by one or more O, NR₇ or CO;
R₇ is hydrogen, C₃-C₃₀cycloalkyl, C₁-C₁₈alkyl, C₁-C₁₀haloalkyl, C₂-C₁₂alkenyl, C₄-C₃₀cycloalkenyl, phenyl-C₁-C₃-alkyl;
or is C₂-C₁₈alkyl which is interrupted by one or more O, S, O(CO) and/or (CO)O;
or is C₃-C₃₀cycloalkyl which is interrupted by one or more O, S, O(CO) and/or (CO)O;
or is C₄-C₃₀cycloalkenyl which is interrupted by one or more O, S, O(CO) and/or (CO)O; or R₇ is Ar, (CO)R₈, (CO)OR₄, (CO)NR₅R₆, and/or SO₂R₈;
wherein R₇ as C₃-C₃₀cycloalkyl, C₁-C₁₈alkyl, C₁-C₁₀haloalkyl, C₂-C₁₂alkenyl, C₄-C₃₀cycloalkenyl, phenyl-C₁-C₃-alkyl, interrupted C₂-C₁₈alkyl, interrupted C₃-C₃₀cycloalkyl, interrupted C₄-C₃₀cycloalkenyl and Ar optionally is substituted by one or more Ar, OH, C₁-C₁₈alkyl, C₁-C₁₀haloalkyl, phenyl-C₁-C₃-alkyl, C₃-C₃₀cycloalkyl, halogen, NO₂, CN, C₁-C₁₈alkoxy, phenoxy, phenoxycarbonyl, phenylthio, phenylthiocarbonyl, NR₅R₆, C₁-C₁₂alkylthio, C₂-C₁₈alkoxycarbonyl, C₂-C₁₀haloalkanoyl, halobenzoyl, C₁-C₁₈alkylsulfonyl, phenylsulfonyl, (4-methylphenyl)sulfonyl, C₁-C₁₈alkylsulfonyloxy, phenylsulfonyloxy, (4-methylphenyl)sulfonyloxy, C₂-C₁₈alkanoyl, C₂-C₁₈alkanoyloxy, benzoyl and/or by benzoyloxy;
R₈ is hydrogen, C₃-C₃₀cycloalkyl, C₁-C₁₈alkyl, C₁-C₁₀haloalkyl, C₂-C₁₂alkenyl, C₄-C₃₀cycloalkenyl, phenyl-C₁-C₃-alkyl, Ar, NR₅R₆;
or is C₂-C₁₈alkyl which is interrupted by one or more O, S, NR, O(CO), (CO)O, (CO)NR₇ and/or NR₇(CO);
or is C₃-C₃₀cycloalkyl which is interrupted by one or more O, S, NR₇, CO, O(CO), (CO)O, (CO)NR₇, and/or NR₇(CO);
or is C₄-C₃₀cycloalkenyl which is interrupted by one or more O, S, NR₇, O(CO), (CO)O, (CO)NR₇, and/or NR₇(CO);
wherein R₈ as C₃-C₃₀cycloalkyl, C₁-C₁₈alkyl, C₁-C₁₀haloalkyl, C₂-C₁₂alkenyl, C₄-C₃₀cycloalkenyl, phenyl-C₁-C₃-alkyl, Ar interrupted C₂-C₁₈alkyl, interrupted C₃-C₃₀cycloalkyl and interrupted C₄-C₃₀cycloalkenyl optionally is substituted by one or more Ar, OH, C₁-C₁₈alkyl, C₁-C₁₀haloalkyl, phenyl-C₁-C₃-alkyl, C₃-C₃₀cycloalkyl, halogen, NO₂, CN, C₁-C₁₈alkoxy, phenoxy, phenoxycarbonyl, phenylthio, phenylthiocarbonyl, NR₅R₆, C₁-C₁₂alkylthio, C₂-C₁₈alkoxycarbonyl, C₂-C₁₀haloalkanoyl, halobenzoyl, C₁-C₁₈alkylsulfonyl, phenylsulfonyl, (4-methylphenyl)sulfonyl, C₁-C₁₈alkylsulfonyloxy, phenylsulfonyloxy, (4-methylphenyl)sulfonyloxy, C₂-C₁₈alkanoyl, C₂-C₁₈alkanoyloxy, benzoyl and/or by benzoyloxy;
Ar is phenyl, biphenyl, fluorenyl, naphthyl, anthracyl, phenanthryl, or heteroaryl, wherein the phenyl, biphenyl, fluorenyl, naphthyl, anthracyl, phenanthryl, or heteroaryl optionally are substituted by one or more C₃-C₃₀cycloalkyl, C₁-C₁₈alkyl, C₁-C₁₀haloalkyl, C₂-C₁₂alkenyl, C₄-C₃₀cycloalkenyl, phenyl-C₁-C₃-alkyl;
or are substituted by C₂-C₁₈alkyl which is interrupted by one or more O, S, NR₇, O(CO), (CO)O, (CO)NR₇ and/or NR₇(CO);
or are substituted by C₃-C₃₀cycloalkyl which is interrupted by one or more O, S, NR₇, O(CO), (CO)O, (CO)NR₇ and/or NR₇(CO);
or are substituted by C₄-C₃₀cycloalkenyl which is interrupted by one or more O, S, NR₇, O(CO), (CO)O, (CO)NR₇, and/or NR₇(CO);
or are substituted by halogen, NO₂, CN, phenyl, biphenyl, naphthyl, heteroaryl, (CO)R₈, (CO)OR₄, (CO)NR₅R₆, O(CO)R₈, O(CO)OR₄, O(CO)NR₅R₆, NR₇(CO)R₈, NR₇(CO)OR₄, OR₄, NR₅R₆, SR₇, SOR₈, SO₂R₈ and/or OSO₂R₈, optionally the substituents C₁-C₁₈alkyl, C₂-C₁₂alkenyl, (CO)R₈, (CO)OR₄, (CO)NR₅R₆, O(CO)R₈, O(CO)OR₄, O(CO)NR₅R₆, NR₇(CO)R₈, NR₇(CO)OR₄, OR₄, NR₅R₆, SR₇, SOR₈, SO₂R₈ and/or OSO₂R₈, form 5-, 6- or 7-membered rings, via the radicals C₁-C₁₈alkyl, C₂-C₁₂alkenyl, R₄, R₅, R₆, R₇ and/or R₈, with further substituents on the phenyl, biphenyl, fluorenyl, naphthyl, anthracyl, phenanthryl or heteroaryl or with one of the carbon atoms of phenyl, biphenyl, fluorenyl, naphthyl, anthracyl, phenanthryl, or heteroaryl;

### X⁻ is

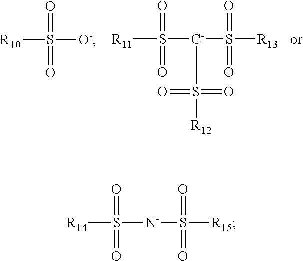

R₁₀ is C₁-C₁₈alkyl, C₁-C₁₀haloalkyl, camphoryl, phenyl-C₁-C₃alkyl, C₃-C₃₀cycloalkyl, Ar;
or is C₂-C₁₈alkyl which is interrupted by one or more O, S, NR₇, O(CO), (CO)O, (CO)NR₇, and/or NR₇(CO);
or is C₂-C₁₀haloalkyl which is interrupted by one or more O, S, NR₇, O(CO), (CO)O, (CO)NR₇, and/or NR₇(CO);
wherein R₁₀ as C₁-C₁₈alkyl, C₁-C₁₀haloalkyl, camphoryl, phenyl-C₁-C₃alkyl, C₃-C₃₀cycloalkyl, Ar, interrupted C₂-C₁₈alkyl or interrupted C₂-C₁₀haloalkyl optionally is substituted by one or more halogen, NO₂, CN, Ar, (CO)R₈, (CO)OR₄, (CO)NR₅R₆, O(CO)R₈, O(CO)OR₄, O(CO)NR₅R₆, NR₇(CO)R₈, NR₇(CO)OR₄, OR₄, NR₅R₆, SR₇, SOR₈, SO₂R₈ and/or OSO₂R₈;
wherein all R₁₀ optionally additionally are substituted by a group having a —O—C-bond or a —O—Si-bond which cleaves upon the action of an acid;
R₁₁, R₁₂ and R₁₃ independently of each other are C₁-C₁₀haloalkyl, Ar;
or are C₂-C₁₀haloalkyl which is interrupted by one or more O, S, NR₇, O(CO), (CO)O, (CO)NR₇, and/or NR₇(CO);
wherein R₁₁, R₁₂ and R₁₃ as C₁-C₁₀haloalkyl, Ar and interrupted C₂-C₁₀haloalkyl optionally are substituted by one or more NO₂, CN, Ar, (CO)R₈, (CO)OR₄, (CO)NR₅R₆, O(CO)R₈, O(CO)OR₄, O(CO)NR₅R₆, NR₇(CO)R₈, NR₇(CO)OR₄, OR₄, NR₅R₆, SR₇, SOR₈, SO₂R₈ and/or OSO₂R₈;
or R₁₁ and R₁₂, together with the

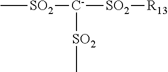

to which they are attached, form a 5-, 6- or 7-membered ring which optionally is interrupted by one or more O, NR₇, or CO;
R₁₄ and R₁₅ independently of each other are C₁-C₁₀haloalkyl, Ar;
or are C₂-C₁₀haloalkyl which is interrupted by one or more O, S, NR₇, O(CO), (CO)O, (CO)NR₇, and/or NR₇(CO);
wherein R₁₄, and R₁₅ as C₁-C₁₀haloalkyl, Ar and interrupted C₂-C₁₀haloalkyl optionally are substituted by one or more NO₂, CN, Ar, (CO)R₈, (CO)OR₄, (CO)NR₅R₆, O(CO)R₈, O(CO)OR₄, O(CO)NR₅R₆, NR₇(CO)R₈, NR₇(CO)OR₄, OR₄, NR₅R₆, SR₇, SOR₈, SO₂R₈ and/or OSO₂R₈;
or R₁₄ and R₁₅, together with —SO₂—N⁻—SO₂— to which they are attached, form a 5-, 6- or 7-membered ring which optionally is interrupted by one or more O, NR₇ or CO and which ring is unsubstituted or substituted by one or more halogen.

The compounds of the formula I are characterized in that two aryl rings of the sulfonium salt form a condensed ring structure with oxygen, sulfur or carbonyl group, and that they have at least one polymerizable ethylenically unsaturated group at the chromophore moiety.

Of interest are in particular compounds of the formula I, wherein

R₁, R₂ and R₃ independently of each other are hydrogen or C₁-C₁₈alkyl;
D₂, D₃ and D₄ independently of each other are a direct bond, O, S, NR₇, CO, (CO)O or (CO)NR₇, C₁-C₁₈alkylene, Ar₁, or C₂-C₁₈alkylene which is interrupted by one or more O, S, NR₇, O(CO) or NR₇(CO);
wherein D₂, D₃ and D₄ as C₁-C₁₈alkylene, interrupted C₂-C₁₈alkylene and Ar₁ optionally are substituted by one or more Ar, OH, C₁-C₁₈alkyl, C₁-C₁₀haloalkyl, halogen, NO₂, CN, C₁-C₁₈alkoxy and/or by phenoxy;
Ar₁ is phenylene, biphenylene or naphthylene; which phenylene, biphenylene or naphthylene optionally are substituted by one or more C₁-C₁₈alkyl, C₁-C₁₀haloalkyl, halogen, NO₂, CN, Ar, OR₄, NR₅R₆ and/or SR₇;
optionally the substituents C₁-C₁₈alkyl, OR₄, NR₅R₆ and/or SR₇ form 5-, 6- or 7-membered rings, via the radicals C₁-C₁₈alkyl, R₄, R₅, R₆ and/or R₇, with further substituents on the phenylene, biphenylene or naphthylene or with one of the carbon atoms of the phenylene, biphenylene or naphthylene;
Ar₂ and Ar₃ independently of each other are phenylene or naphthylene, which phenylene or naphthylene optionally are substituted by one or more C₁-C₁₈alkyl, C₁-C₁₀haloalkyl, halogen, NO₂, CN, Ar, OR₄, NR₅R₆ and/or SR₇;
optionally the substituents C₁-C₁₈alkyl, OR₄, NR₅R₆ and/or SR₇ form 5-, 6- or 7-membered rings, via the radicals C₁-C₁₈alkyl, R₄, R₅, R₆ and/or R₇, with further substituents on the phenylene or naphthylene or with one of the carbon atoms of the phenylene or naphthylene;
R₄ is hydrogen, C₁-C₁₈alkyl, Ar, (CO)R₈ or SO₂R₈;
R₅ and R₆ independently of each other are hydrogen, C₁-C₁₈alkyl, Ar, (CO)R₈ or SO₂R₈;
R₇ is hydrogen, C₁-C₁₈alkyl, Ar, (CO)R₈ or SO₂R₈;
R₈ is hydrogen, C₁-C₁₈alkyl or Ar;
Ar is phenyl, biphenyl or naphthyl, which phenyl, biphenyl or naphthyl optionally are substituted by one or more C₁-C₁₈alkyl, halogen, NO₂, CN, OR₄, NR₅R₆ and/or SR₇; optionally the substituents C₁-C₁₈alkyl, OR₄, NR₅R₆ and/or SR₇, form 5-, 6- or 7-membered rings, via the radicals C₁-C₁₈alkyl, R₄, R₅, R₆ and/or R₇, with further substituents on the phenyl, biphenyl or naphthyl or with one of the carbon atoms of the phenyl, biphenyl or naphthyl;
R₁₀ is C₁-C₁₈alkyl, C₁-C₁₀haloalkyl, camphoryl, phenyl-C₁-C₃alkyl, Ar; or is C₂-C₁₀haloalkyl which is interrupted by one or more O and/or O(CO);
wherein the C₁-C₁₈alkyl, C₁-C₁₀haloalkyl, camphoryl, phenyl-C₁-C₃alkyl, Ar or interrupted C₂-C₁₀haloalkyl optionally are substituted by one or more (CO)OR₄, O(CO)R₈, O(CO)OR₄ and/or OR₄;
R₁₁, R₁₂ and R₁₃ independently of each other are C₁-C₁₀haloalkyl; and
R₁₄ and R₁₅ independently of each other are C₁-C₁₀haloalkyl;
or R₁₄ and R₁₅, together with —SO₂—N⁻—SO₂— to which they are attached, form a 5-, 6- or 7-membered ring.

Especially interesting are compounds of the formula I described above, wherein

R₁, R₂ and R₃ independently of each other are hydrogen or C₁-C₁₈alkyl;
D₂, D₃ and D₄ independently of each other are a direct bond, O, S, CO, (CO)O, (CO)NR₇, C₁-C₁₈alkylene or Ar₁;
Ar₁ is phenylene, biphenylene or naphthylene; which phenylene, biphenylene or naphthylene optionally are substituted by one or more C₁-C₁₈alkyl, halogen and/or OR₄;
Ar₂ and Ar₃ independently of each other are phenylene, which optionally is substituted by one or more C₁-C₁₈alkyl, halogen and/or OR₄;
R₄ is hydrogen, C₁-C₁₈alkyl, (CO)R₈ and/or SO₂R₈;
R₇ is hydrogen or C₁-C₁₈alkyl;
Ar is phenyl, which optionally is substituted by one or more C₁-C₁₈alkyl, halogen, NO₂ and/or OR₄;
R₁₀ is C₁-C₁₈alkyl, C₁-C₁₀haloalkyl, camphoryl, phenyl-C₁-C₃alkyl, Ar;
or is C₂-C₁₀haloalkyl which is interrupted by one or more O and/or O(CO);
which C₁-C₁₈alkyl, C₁-C₁₀haloalkyl, camphoryl, phenyl-C₁-C₃alkyl, Ar and interrupted C₂-C₁₀haloalkyl optionally are substituted by one or more (CO)OR₄, O(CO)R₈, O(CO)OR₄ and/or OR₄;
R₁₁, R₁₂ and R₁₃ independently of each other are C₁-C₁₀haloalkyl;
R₁₄ and R₁₅ independently of each other are C₁-C₁₀haloalkyl;
or R₁₄ and R₁₅, together with —SO₂—N⁻—SO₂— to which they are attached, form a 5-, 6- or 7-membered ring.

A particular subject of the invention are compounds of the formula I, wherein

R₁, R₂ and R₃ independently of each other are hydrogen or C₁-C₁₈alkyl;

### Y is O or S;

D₂, D₃ and D₄ independently of each other are O, O(CO), C₁-C₁₈alkylene or Ar₁;
Ar₁ is phenylene,
Ar₂ and Ar₃ are phenylene;
Ar is phenyl;

### X⁻ is

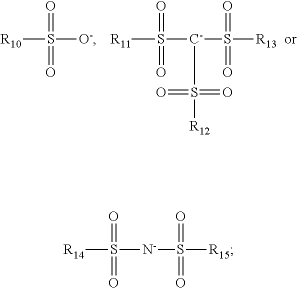

R₁₀ is C₁-C₁₀haloalkyl;
R₁₁, R₁₂ and R₁₃ independently of each other are C₁-C₁₀haloalkyl; and
R₁₄ and R₁₅ independently of each other are C₁-C₁₀haloalkyl;
or R₁₄ and R₁₅, together with —SO₂—N⁻—SO₂— to which they are attached, form a 5-, 6- or 7-membered ring which is substituted by one or more halogen.

The compounds of the formula I can be polymerized, either with one another or with other components comprising ethylenically unsaturated polymerizable groups.

Subject of the invention therefore also is a polymer comprising at least one repeating unit derived from the compound of the formula I as described above.

Interesting polymers are such additionally to the at least one repeating unit derived from the compound of the formula I, comprising one or more identical or different repeating units derived from ethylenically unsaturated compounds of formula II

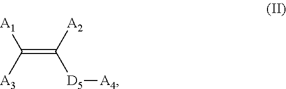

wherein
A₁, A₂ and A₃ independently of each other are hydrogen, halogen, CN, C₁-C₁₈alkyl, C₁-C₁₀haloalkyl, (CO)R₈, (CO)OR₄, (CO)NR₅R₆ or C₁-C₁₈alkyl which is substituted by OR₄;
A₄ is C₁-C₁₈alkyl, C₂-C₁₈alkyl which is interrupted by one or more O, S, NR₇, CO, SO and/or SO₂,
C₃-C₃₀cycloalkyl, C₃-C₃₀cycloalkyl which is interrupted by one or more O, S, NR₇, CO, SO and/or SO₂,
C₂-C₁₂alkenyl, C₂-C₁₂alkenyl which is interrupted by one or more O, S, NR₇, CO, SO and/or SO₂,
C₄-C₃₀cycloalkenyl, C₄-C₃₀cycloalkenyl which is interrupted by one or more O, S, NR₇, CO, SO and/or SO₂,
wherein the groups C₁-C₁₈alkyl, interrupted C₂-C₁₈alkyl, C₃-C₃₀cycloalkyl, interrupted C₃-C₃₀cycloalkyl, C₂-C₁₂alkenyl, interrupted C₂-C₁₂alkenyl, C₄-C₃₀cycloalkenyl and interrupted C₄-C₃₀cycloalkenyl optionally are substituted by one or more Ar, OR₄, (CO)OR₄, O(CO)R₈, halogen, NO₂, CN, NR₅R₆, C₁-C₁₂alkylthio, C₁-C₁₈alkylsulfonyloxy, phenylsulfonyloxy, and/or (4-methylphenyl)sulfonyloxy;
or A₄ is hydrogen, halogen, NO₂, CN, Ar, (CO)R₈, (CO)OR₄, (CO)NR₅R₆, O(CO)R₈, O(CO)OR₄, O(CO)NR₅R₆, NR₇(CO)R₈, NR₇(CO)OR₄, OR₄, NR₅R₆, SR₇, SOR₈, SO₂R₈ and/or OSO₂R₈;
D₅ is a direct bond, O, CO, (CO)O, (CO)S, (CO)NR₇, SO₂, OSO₂, C₁-C₁₈alkylene or Ar¹; optionally A₃ and D₅, together with the ethylenically unsaturated double bond to which they are attached form C₃-C₃₀cycloalkenyl which optionally is interrupted by one or more O, S, NR₇, CO, SO and/or SO₂;
or optionally the radicals A₂ and D₅ together with the carbon atom of the ethylenically unsaturated double bond to which they are attached form C₃-C₃₀cycloalkyl which optionally is interrupted by one or more O, S, N, NR₇, CO, SO and/or SO₂; and
R₄, R₅, R₆, R₇, R₈, Ar and Ar₁ are as defined above.
R₁ and R₂ are for example hydrogen or C₁-C₁₈alkyl, in particular are hydrogen or C₁-C₈alkyl, especially are hydrogen.
R₃ is for example hydrogen or C₁-C₁₈alkyl, in particular is hydrogen or C₁-C₈alkyl, especially is hydrogen.
Y is in particular S or O.
D₂ is for example C₁-C₁₈alkylene, O(CO) or An, e.g. C₁-C₁₈alkylene, O(CO) or phenylene, especially O(CO) or Ar₁, in particular Ar₁, preferably phenylene.
D₃ is for example C₁-C₁₈alkylene, e.g. C₁-C₁₂alkylene, in particular C₁-C₄alkylene, especially methylene or ethylene, preferably methylene.
D₄ preferably is O.
Ar₁ is for example phenylene, naphthylene or biphenylene, e.g. phenylene or naphthylene, or is phenylene or biphenylene, in particular phenylene.
Ar₂ and Ar₃ in particular are identical, and are for example phenylene or naphthylene, preferably phenylene. Preferably Ar₂ and Ar₃ are unsubstituted.
R₄ is for example C₃-C₃₀cycloalkyl which is interrupted by O(CO), or is Ar; in particular C₅-C₈cycloalkyl which is interrupted by O(CO), or is Ar, in particular C₅-C₈cycloalkyl which is interrupted by O(CO), or is phenyl.
R₄ in formula (II) is for example hydrogen or C₁-C₁₈alkyl, in particular hydrogen.
R₅ and R₆ are for example independently of each other hydrogen or C₁-C₄alkyl or together with the N-atom to which they are attached form a 6-membered ring which optionally is interrupted by O or NR₇. R₅ and R₆ especially together with the N-atom to which they are attached form a 6-membered ring which optionally is interrupted by O, preferably R₅ and R₆ especially together with the N-atom to which they are attached form a morpholino ring.
R₈ is for example NR₅R₆ or is C₃-C₃₀cycloalkyl which is interrupted by CO or O(CO).
R₈ in formula (II) is for example C₃-C₃₀cycloalkyl or C₁-C₈alkyl (which as defined below includes C₃-C₃₀cycloalkyl substituted by C₁-C₂₄alkyl), in particular is C₅-C₁₅cycloalkyl which is substituted by C₁-C₈alkyl, preferably is

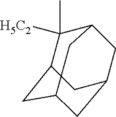

or R₈ is C₁-C₈alkyl.
Ar in formula (I) in particular is phenyl.
Ar in formula (II) is for example phenyl which is substituted by O(CO)R₈ or OR₄.

### X is

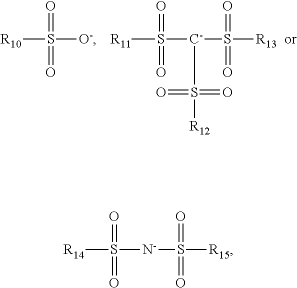

especially

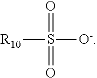

R₁₀ is for example C₁-C₁₀haloalkyl or C₃-C₃₀cycloalkyl which C₁-C₁₀haloalkyl and C₃-C₃₀cycloalkyl are unsubstituted or are substituted by for example (CO)R₈, (CO)OR₄, O(CO)R₈, OR₄ or SO₂R₈; R₁₀ in particular is C₁-C₁₀haloalkyl.
R₁₁, R₁₂, R₁₃ in particular are C₁-C₁₀haloalkyl.
R₁₄ and R₁₅ are for example C₁-C₁₀haloalkyl or together with —SO₂—N⁻—SO₂— to which they are attached, form a 6-membered ring which is unsubstituted or substituted by one or more halogen, preferably R₁₄ and R₁₅ together with —SO₂—N⁻—SO₂— to which they are attached, form a 6-membered ring which is unsubstituted or substituted by one or more halogen, in particular form a ring which is substituted by one or more halogen.
D₅ is in particular a direct bond or is (CO)OR₈, wherein R₈ os

Interesting are such compounds of the formula (I) which have a structure of the formula (Ia)

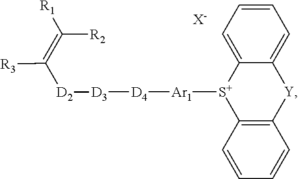

wherein R₁, R₂, R₃, D₂, D₃, D₄, An and X are as defined above;
as well as such compounds of the formula (I) which have a structure of the formula (Ib)

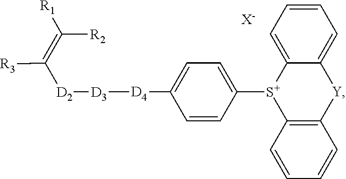

wherein R₁, R₂, R₃, D₂, D₃, D₄ and X are as defined above.

Interesting are such polymers as defined above which comprise

(i) repeating units of formula I; and
(ii) repeating units of the formula (II) wherein
A₁, A₂ and A₃ are hydrogen or C₁-C₄alkyl, in particular hydrogen,
D₅ is a direct bond,
A₄ is Ar,
Ar is phenyl which is substituted by OCOR₈ or OR₄,
R₄ is hydrogen or C₁-C₄alkyl, in particular hydrogen, and
R₈ is C₁-C₄alkyl; and
   (iii) repeating units of formula (II), wherein
A₁ and A₂ are hydrogen,
A₃ is hydrogen or C₁-C₄alkyl, in particular methyl,
D₅ is (CO)O,
A₄ is C₃-C₃₀cycloalkyl (in particular C₅-C₁₅cycloalkyl which is substituted by C₁-C₈alkyl), especially

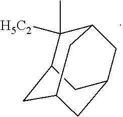

In particular interesting are the compounds of formula (I) as given in the example 1 and 2, as well as the compounds the following formulae (a)-(m):

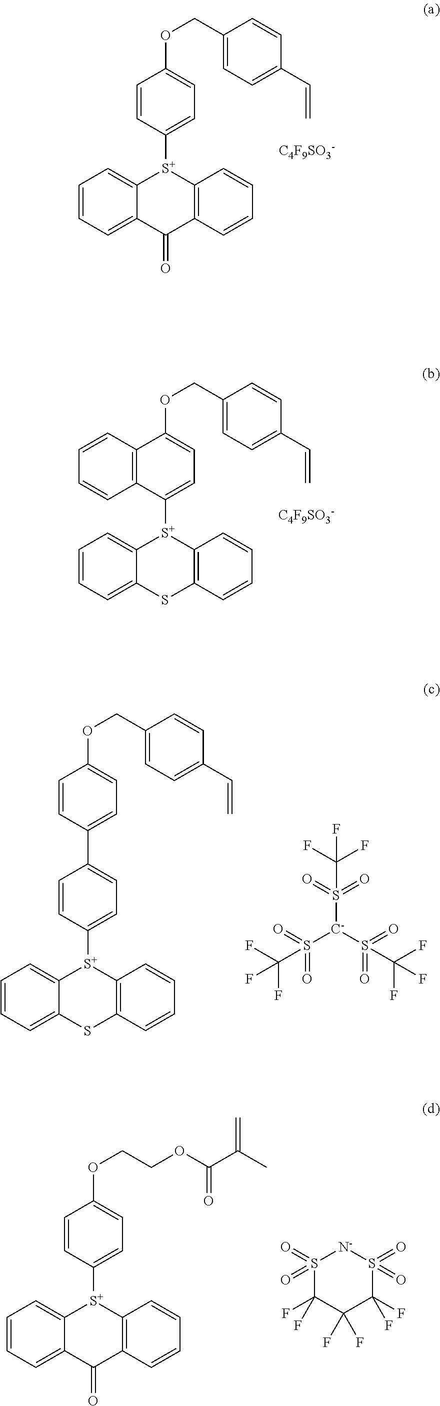

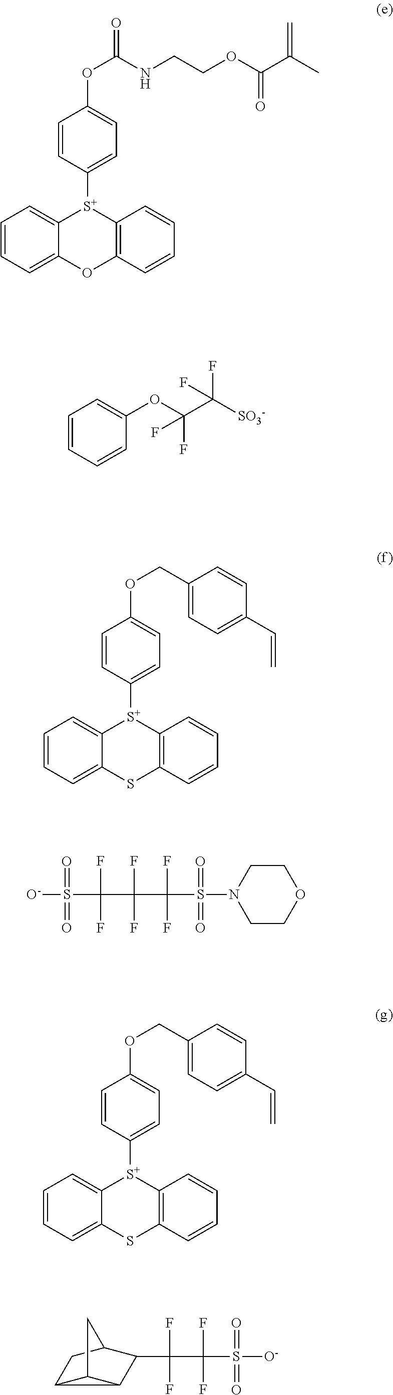

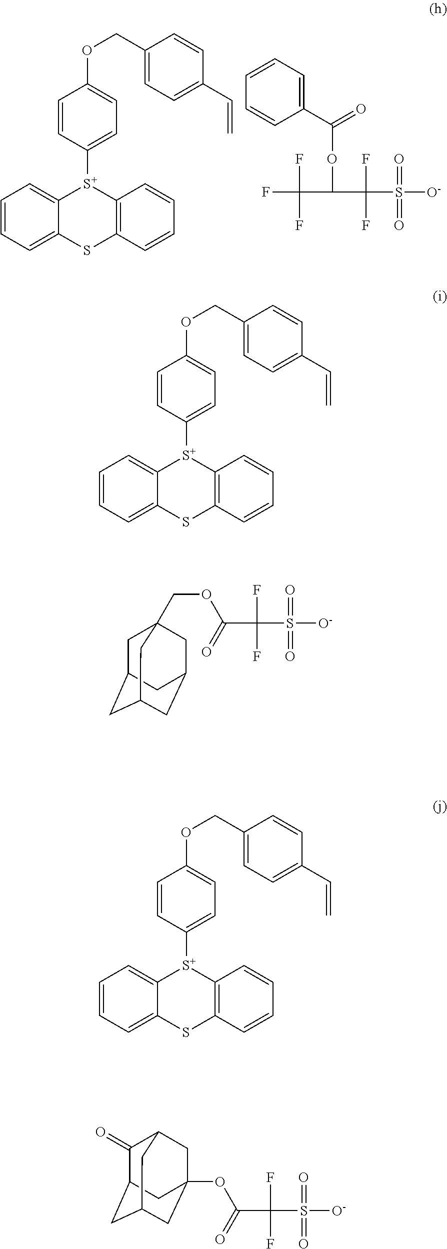

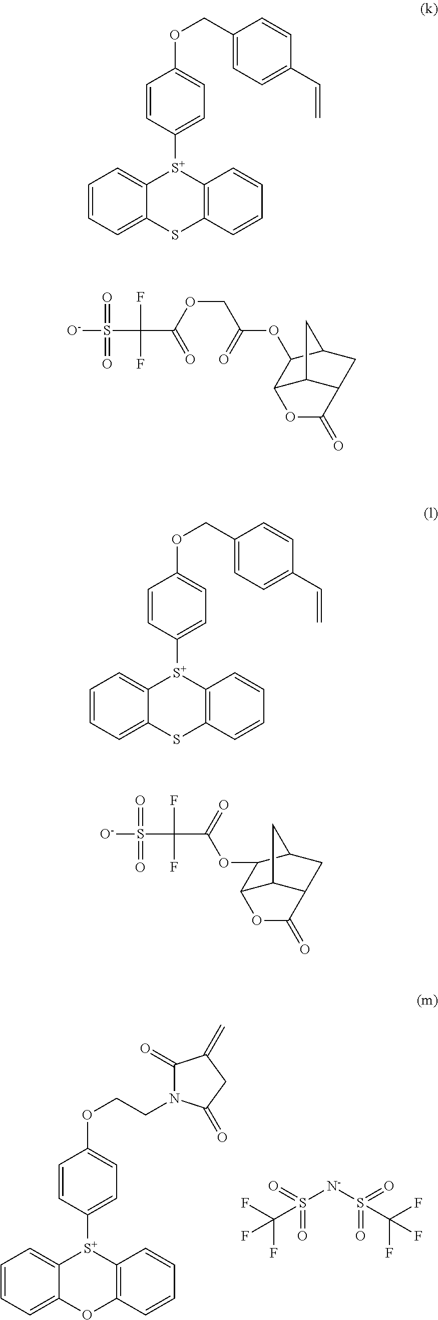

In particular interesting are the repeating units of formula (II) as given in the example 3, 4 and 5:

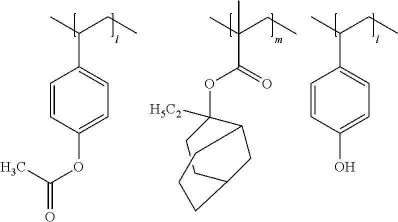

C₁-C₁₈alkyl is linear or branched and is, for example, C₁-C₁₆—, C₁-C₁₂—, C₁-C₈—, C₁-C₆— or C₁-C₄-alkyl. Examples are methyl, ethyl, propyl, isopropyl, n-butyl, sec-butyl, isobutyl, tert-butyl, pentyl, hexyl, heptyl, 2,4,4-trimethylpentyl, 2-ethylhexyl, octyl, nonyl, decyl, undecyl, dodecyl, tetradecyl, pentadecyl, hexadecyl, heptadecyl and octadecyl, preferably C₁-C₄alkyl, such as methyl, isopropyl or butyl.

C₂-C₁₈alkyl which is interrupted by one or more O, S, NR₇, O(CO) and/or NR₇(CO) is, for example, interrupted from one to five times, for example from one to three times or once or twice, by non-successive O, S, NR₇, O(CO) and/or NR₇(CO). Accordingly, resulting structural units are for example: O(CH₂)₂OH, O(CH₂)₂OCH₃, O(CH₂CH₂O)₂CH₂CH₃, CH₂—O—CH₃, CH₂CH₂—O—CH₂CH₃, [CH₂CH₂O]_{y}—CH₃, wherein y=1-5, (CH₂CH₂O)₅CH₂CH₃, CH₂—CH(CH₃)—O—CH₂—CH₂CH₃, CH₂—CH(CH₃)—O—CH₂—CH₃, S(CH₂)₂SCH₃, (CH₂)₂NHCH₃, (CH₂)₂O(CO)CH₃, (CH₂)₂(CO)OCH₃ or (CH₂)₂NH(CO)CH₃.

If, in the context of the present invention a group, e.g. alkyl or alkylene, is interrupted by one or more defined radicals, e.g. O, S, NR₇, O(CO) and/or NR₇(CO), the “interrupting” radicals not only are meant to be situated in between the interrupted group, for example the alkyl or alkylene, but also are meant to be terminal.

C₃-C₃₀cycloalkyl is a mono- or polycyclic aliphatic ring, for example a mono-, bi- or tricyclic aliphatic ring, e.g. C₃-C₂₀-, C₃-C₁₈—, C₃-C₁₂—, C₃-C₁₀cycloalkyl. C₃-C₃₀cycloalkyl in the context of the present application is to be understood as alkyl which at least comprises one ring, i.e. also carbocyclic aliphatic rings, which are substituted by C₁-C₂₄alkyl are covered by this definition. Examples of monocyclic rings are cyclopropyl, cyclobutyl, cyclopentyl, cyclohexyl, or cycloheptyl, especially cyclopentyl and cyclohexyl. Further examples are structures like

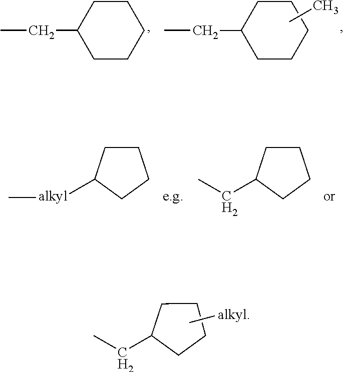

Examples of polycyclic rings are perhydroanthracyl, perhydrophenyathryl, perhydronaphthyl, perhydrofluorenyl, perhydrochrysenyl, perhydropicenyl, adamantyl, bicyclo[1.1.1]pentyl, bicyclo[4.2.2]decyl, bicyclo[2.2.2]octyl, bicyclo[3.3.2]decyl, bicyclo[4.3.2]undecyl, bicyclo[4.3.3]dodecyl, bicyclo[3.3.3]undecyl, bicyclo[4.3.1]decyl, bicyclo[4.2.1]nonyl, bicyclo[3.3.1]nonyl, bicyclo[3.2.1]octyl,

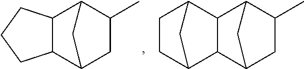

and the like. Also alkyl-substituted polycyclic and bridged rings are meant to be covered by the definition “cycloalkyl” in the context of the present invention, e.g.

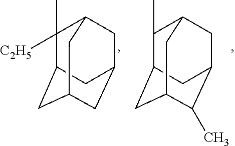

etc .

Also “spiro”-cycloalkyl compounds are covered by the definition C₃-C₃₀cycloalkyl in the present context, e.g. spiro[5.2]octyl, spiro[5.4]decyl, spiro[5.5]undecyl. More examples of polycyclic cycloalkyl groups, which are subject of the respective definition in the compounds of the present invention are listed in EP 878738, page 11 and 12, wherein to the formulae (1)-(46) a bond to achieve the “yl” has to be added. The person skilled in the art is aware of this fact.

In general, the cycloaliphatic rings may form repeating structural units.

C₃-C₃₀cycloalkyl which is interrupted by one or more O, S, NR₇, O(CO), (CO)O, (CO)NR₇, and/or NR₇(CO) is a mono- or polycyclic aliphatic ring which is interrupted by one or more O, S, NR₇, O(CO), (CO)O, (CO)NR₇, and/or NR₇(CO), for example,

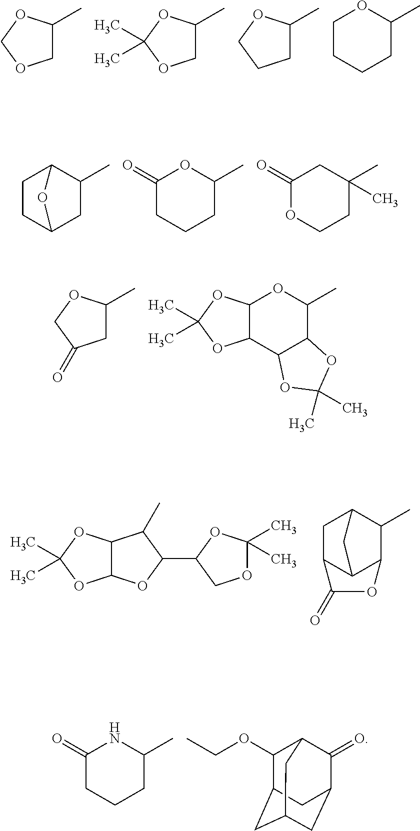

C₂-C₁₂alkenyl radicals are for example mono- or polyunsaturated, linear or branched and are for example C₂-C₈—, C₂-C₆— or C₂-C₄alkenyl. Examples are allyl, methallyl, vinyl, 1,1-dimethylallyl, 1-butenyl, 3-butenyl, 2-butenyl, 1,3-pentadienyl, 5-hexenyl or 7-octenyl, especially allyl or vinyl.

C₄-C₁₀cycloalkenyl is a mono- or polycyclic and mono- or polyunsaturated ring, for example a mono-, bi-, tri- or tetracyclic mono- or polyunsaturated ring, e.g. C₄-C₂₀—, C₄-C₁₈—, C₄-C₁₂—, C₄-C₁₀cycloalkenyl. Examples of cycloalkenyl are cyclobutenyl, cyclopentenyl, cyclohexenyl, cycloheptenyl. Also bridged alkenyl groups are covered by the above definition, for example

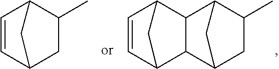

etc., especially cyclopentenyl, cyclohexenyl,

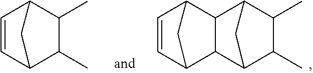

C₄-C₃₀cycloalkenyl which is interrupted by one or more O, S, NR₇, O(CO) and/or NR₇(CO) is a mono- or polycyclic and mono- or polyunsaturated ring, which is interrupted by one or more O, S, NR₇, O(CO) and/or NR₇(CO), for example,

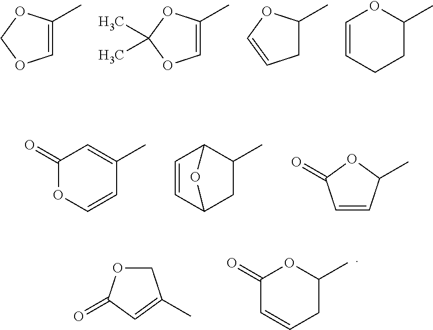

C₁-C₁₈alkylene is linear or branched alkylene. Examples are ethylene, propylene, butylene, pentylene, hexylene.

C₂-C₁₈alkylene which is interrupted by one or more O, S, NR₇, O(CO), (CO)O, (CO)NR₇, or NR₇(CO), is interrupted, for example, from one to five times, for example from one to three times or once or twice, by “non-successive O”, by, S, NR₇, O(CO), (CO)O, (CO)NR₇, or NR₇(CO). “Interrupted” in this definition in the context of the present application is also meant to comprise C₂-C₁₈alkylene having one or more of said defined groups attached at one end or both ends of the alkyl chain. Accordingly, resulting structural units are for example: —O(CH₂)₂—, —O(CH₂)₂OCH₂—, —O(CH₂CH₂O)₂— —S(CH₂)₂— —(CH₂)₂NH—, —(CH₂)₂O(CO)CH₂—, —CH₂CH₂NHCO—.

C₃-C₃₀cycloalkylene is a mono- or polycyclic aliphatic ring, for example a mono-, bi- or tricyclic aliphatic ring, e.g. C₃-C₂₀-, C₃-C₁₈—, C₃-C₁₂—, C₃-C₁₀cycloalkylene. Examples of monocyclic rings are cyclopropylene, cyclobutylene, cyclopentylene, cyclohexylene, or cycloheptylene. Examples of polycyclic rings are perhydroanthracylene, perhydrophenyathrylene, perhydro-naphthylene, perhydrofluorenylene, perhydrochrysenylene, perhydropicenylene, adamantylene, bicyclo[1.1.1]pentylene, bicyclo[4.2.2]decylene, bicyclo[2.2.2]octylene, bicyclo[3.3.2]decylene, bicyclo[4.3.2]undecylene, bicyclo[4.3.3]dodecylene, bicyclo[3.3.3]undecylene, bicyclo[4.3.1]decylene, bicyclo[4.2.1]nonylene, bicyclo[3.3.1]nonylene, bicyclo[3.2.1]octylene,

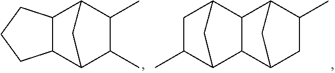

and the like. Also “spiro”-cycloalkylene compounds are covered by the definition C₃-C₃₀cycloalkylene in the present context, e.g. spiro[5.2]octylene, spiro[5.4]decylene, spiro[5.5]undecylene. More examples of polycyclic cycloalkylene groups, which are subject of the respective definition in the compounds of the present invention are listed in EP878738, page 11 and 12, wherein to the formulae (1)-(46) two bonds to achieve the “ylene” has to be added. The person skilled in the art is aware of this fact.

C₃-C₃₀cycloalkylene which is interrupted by one or more O, S, NR₇, O(CO), (CO)O, (CO)NR₇, or NR₇(CO), is a mono- or polycyclic aliphatic ring which is interrupted by one or more O, S, NR₇, O(CO), (CO)O, (CO)NR₇, or NR₇(CO), for example,

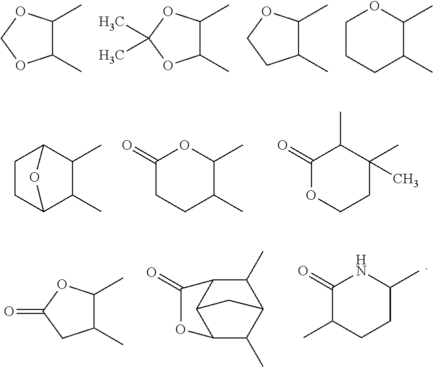

C₂-C₁₂alkenylene radicals are for example mono- or polyunsaturated, linear or branched and are for example C₂-C₈—, C₂-C₆— or C₂-C₄alkenylene. Examples are —CH═CHCH₂—, —CH═C(CH₃)CH₂—, —CH═C(CH₃)—,

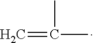

C₄-C₃₀cycloalkenylene is a mono- or polycyclic and mono- or polyunsaturated ring, for example a mono-, bi-, tri- or tetracyclic mono- or polyunsaturated ring, e.g. C₄-C₂₀-, C₄-C₁₈—, C₄-C₁₂—, C₄-C₁₀cycloalkenylene. Examples are

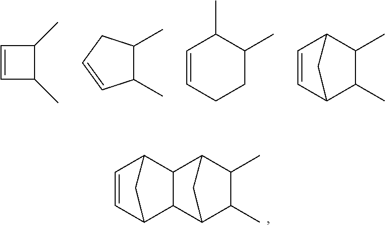

etc.

C₄-C₃₀cycloalkenylene which is interrupted by one or more O, S, NR₇, O(CO), or NR₇(CO), is a mono- or polycyclic and mono- or polyunsaturated ring, which is interrupted by one or more O, S, NR₇, O(CO), (CO)O, (CO)NR₇, or NR₇(CO), for example

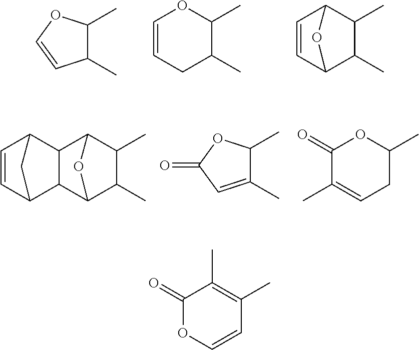

etc.

Substituted phenyl carries from one to five, for example one, two or three, especially one or two, substituents on the phenyl ring. The substitution is preferably in the 4-, 3,4-, 3,5- or 3,4,5-position of the phenyl ring.

When the radicals phenyl, biphenyl, naphthyl, fluorenyl, phenanthryl, anthracyl and heteroaryl are substituted by one or more radicals, they are, for example, mono- to penta-substituted, for example mono-, di- or tri-substituted, especially mono- or di-substituted.

When Ar is phenyl, biphenyl, fluorenyl, naphthyl, anthracyl, phenanthryl, or heteroaryl substituted by one or more C₁-C₁₈alkyl, C₂-C₁₂alkenyl, (CO)R₈, (CO)OR₄, (CO)NR₅R₆, O(CO)R₈, O(CO)OR₄, O(CO)NR₅R₆, NR₇(CO)R₈, NR₇(CO)OR₄, OR₄, NR₅R₆, SR₇, SOR₈, SO₂R₈ and/or OSO₂R₈ and the substituents C₁-C₁₈alkyl, C₂-C₁₂alkenyl, (CO)R₈, (CO)OR₄, (CO)NR₅R₆, O(CO)R₈, O(CO)OR₄, O(CO)NR₅R₆, NR₇(CO)R₈, NR₇(CO)OR₄, OR₄, NR₅R₆, SR₇, SOR₈, SO₂R₈ and/or OSO₂R₈, form 5-, 6- or 7-membered rings, via the radicals C₁-C₁₈alkyl, C₂-C₁₂alkenyl, R₄, R₅, R₆, R₇ and/or R₈, with further substituents on the phenyl, biphenyl, fluorenyl, naphthyl, anthracyl, phenanthryl, or heteroaryl or with one of the carbon atoms of the phenyl, biphenyl, fluorenyl, naphthyl, anthracyl, phenanthryl, or heteroaryl, for example the following structural units are obtained

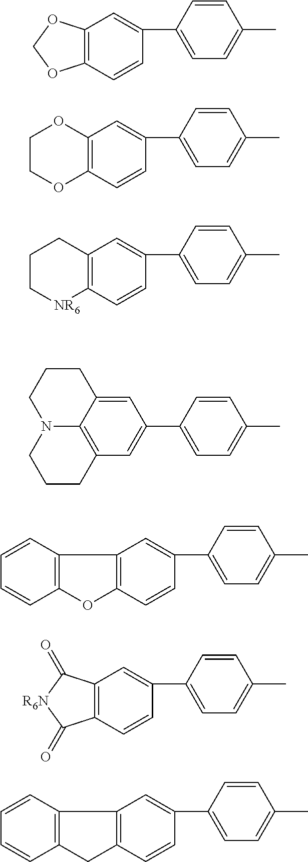

etc.

If in Ar the substituents C₁-C₁₈alkyl form alkylene bridges from one carbon atom of the biphenyl, naphthyl, or fluorenyl ring to another carbon atom of said ring, in particular ethylene, propylene and butylene bridges are formed and for example the following structures are obtained

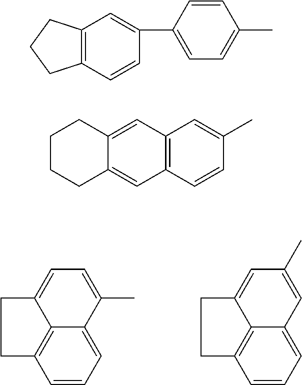

etc. The definition according to the present application in this connection also is intended to cover branched alkylene bridges:

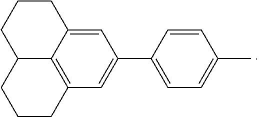

In case said alkylene bridges are condensed with further phenyl rings for example the following structure is given

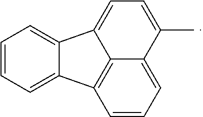

When Ar₁ is phenylene, biphenylene, naphthylene,

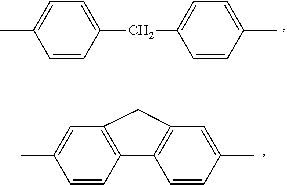

heteroarylene, oxydiphenylene or

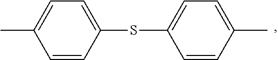

all of which are substituted by one or more C₁-C₁₈alkyl, C₂-C₁₂alkenyl, (CO)R₈, (CO)OR₄, (CO)NR₅R₆, O(CO)R₈, O(CO)OR₄, O(CO)NR₅R₆, NR₇(CO)R₈, NR₇(CO)OR₄, OR₄, NR₅R₆, SR₇, SOR₈, SO₂R₈ and/or OSO₂R₈, and the substituents C₁-C₁₈alkyl, C₂-C₁₂alkenyl, (CO)R₈, (CO)OR₄, (CO)NR₅R₆, O(CO)R₈, O(CO)OR₄, O(CO)NR₅R₆, NR₇(CO)R₈, NR₇(CO)OR₄, OR₄, NR₅R₆, SR₇, SOR₈, SO₂R₈ and/or OSO₂R₈ form 5-, 6- or 7-membered rings, via the radicals C₁-C₁₈alkyl, C₂-C₁₂alkenyl, R₄, R₅, R₆, R₇ and/or R₈, with further substituents on the phenylene, biphenylene, naphthylene,

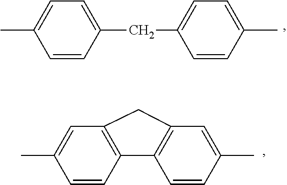

heteroarylene, oxydiphenylene or

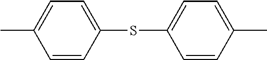

or with one of the carbon atoms of the phenylene, biphenylene, naphthylene,

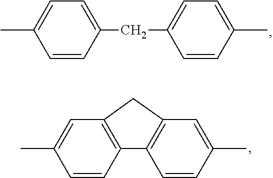

heteroarylene,

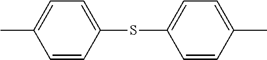

or oxydiphenylene, for example the following structural units are obtained

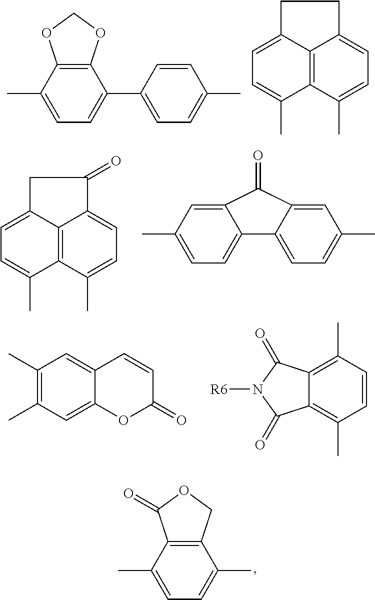

etc.

Camphoryl, 10-camphoryl, are camphor-10-yl, namely

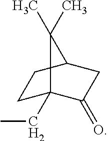

C₂-C₁₈alkanoyl is e.g. C₂-C₁₂, C₂-C₈—, C₂-C₆— or C₂-C₄alkanoyl, wherein the alkyl moiety is linear or branched. Examples are acetyl, propionyl, butanoyl or hexanoyl, especially acetyl.

C₁-C₁₈alkoxy is e.g. C₁-C₁₂—, C₁-C₈—, C₁-C₆—, C₁-C₄alkoxy, and is linear or branched. Examples are methoxy, ethoxy, propoxy, n-butoxy, t-butoxy, octyloxy and dodecyloxy.

In C₁-C₁₂alkylthio the alkyl moiety is for example linear or branched. Examples are methylthio, ethylthio, propylthio or butylhtio.

C₂-C₁₈alkoxycarbonyl is (C₁-C₁₇alkyl)-O—C(O)—, wherein C₁-C₁₇alkyl is linear or branched and is as defined above up to the appropriate number of carbon atoms. Examples are C₂-C₁₀—, C₂-C₈—, C₂-C₆— or C₂-C₄alkoxycarbonyl, such as methoxycarbonyl, ethoxycarbonyl, propoxycarbonyl, butoxycarbonyl or pentoxycarbonyl.

C₁-C₁₀haloalkyl are for example C₁-C₈—, C₁-C₆— or C₁-C₄-alkyl mono- or poly-substituted by halogen, the alkyl moieties being, for example, as defined above. There are, for example, from 1 to 23 halogen substituents at the alkyl radical. Examples are chloromethyl, trichloromethyl, trifluoromethyl, nonafluorobutyl or 2-bromopropyl, especially trifluoromethyl or trichloromethyl. Preferred is C₁-C₁₀fluoroalkyl.

C₂-C₁₀haloalkanoyl is (C₁-C₉haloalkyl)-C(O)—, wherein C₁-C₉haloalkyl is as defined above up to the appropriate number of carbon atoms. Examples are chloroacetyl, trichloroacetyl, trifluoroacetyl, pentafluoropropionyl, perfluorooctanoyl, or 2-bromopropionyl, especially trifluoroacetyl or trichloroacetyl.

Halobenzoyl is benzoyl which is mono- or poly-substituted by halogen and/or C₁-C₄haloalkyl, C₁-C₄-haloalkyl being as defined above. Examples are pentafluorobenzoyl, trichlorobenzoyl, trifluoromethylbenzoyl, especially pentafluorobenzoyl.

Halogen is fluorine, chlorine, bromine or iodine, especially chlorine or fluorine, preferably fluorine.

Phenyl-C₁-C₃alkyl is, for example, benzyl, 2-phenylethyl, 3-phenylpropyl, α-methylbenzyl or α,α-dimethylbenzyl, especially benzyl.

If R₅ and R₆ together with the nitrogen atom to which they are bonded form a 5-, 6- or 7-membered ring that optionally is interrupted by O, NR₇ or CO, for example the following structures are obtained

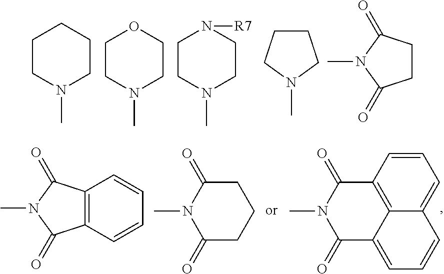

etc.

The definition C₁-C₁₈alkylsulfonyl, refers to the corresponding radical C₁-C₁₈alkyl, as described in detail above, being linked to a sulfonyl group (—SO₂—). Accordingly, also phenylsulfonyl and (4-methylphenyl)sulfonyl refer to the corresponding radicals linked to a sulfonyl group.

C₂-C₁₈alkanoyloxy is (C₁-C₁₇alkyl)-C(O)—O—, wherein C₁-C₁₇alkyl is linear or branched and is as defined above up to the appropriate number of carbon atoms. Examples are C₂-C₁₀—, C₂-C₈—, C₂-C₆— or C₂-C₄alkanoyloxy, such as acetyloxy, ethanoyloxy, propanoyloxy, butanoyloxy or hexanoyloxy.

C₁-C₁₈alkylsulfonyloxy is (C₁-C₁₈alkyl)-S(O)₂—O—, wherein C₁-C₁₈alkyl is linear or branched and is as defined above up to the appropriate number of carbon atoms. Examples are C₁-C₁₀—, C₁-C₈—, C₁-C₆— or C₁-C₄alkylsulfonyloxy, such as methanesulfonyloxy, propanesulfonyloxy or hexanesulfonyloxy.

Accordingly, also phenylsulfonyloxy and (4-methylphenyl)sulfonyloxy refer to the corresponding radicals linked to a —S(O)₂—O— group.

In the present application, the term “heteroaryl” denotes unsubstituted and substituted radicals, for example 3-thienyl, 2-thienyl,

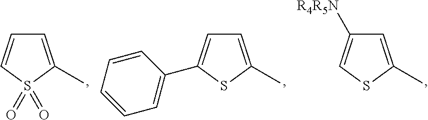

wherein R₄ and R₅ are as defined above, thianthrenyl, isobenzofuranyl, xanthenyl, phenoxathiinyl,

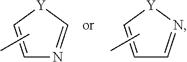

wherein Y is S, O or NR₆ and R₆ is as defined above. Examples thereof are pyrazolyl, thiazolyl, oxazolyl, isothiazolyl or isoxazolyl. Also included are, for example, furyl, pyrrolyl, 1,2,4-triazolyl,

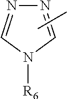

or 5-membered ring heterocycles having a fused-on aromatic group, for example benzimidazolyl, benzothienyl, benzofuranyl, benzoxazolyl and benzothiazolyl.

Other examples of “heteroaryls” are pyridyl, especially 3-pyridyl,

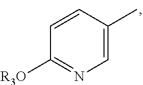

wherein R₃ is as defined above, pyrimidinyl, pyrazinyl, 1,3,5-triazinyl, 2,4-, 2,2- or 2,3-diazinyl, indolizinyl, isoindolyl, indolyl, indazolyl, purinyl, isoquinolyl, quinolyl, phenoxazinyl or phenazinyl. In this Application, the term “heteroaryl” also denotes the radicals thioxanthyl, xanthyl,

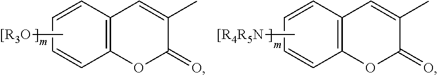

wherein m is 0 or 1 and R₃, R₄, R₅ are as defined above,

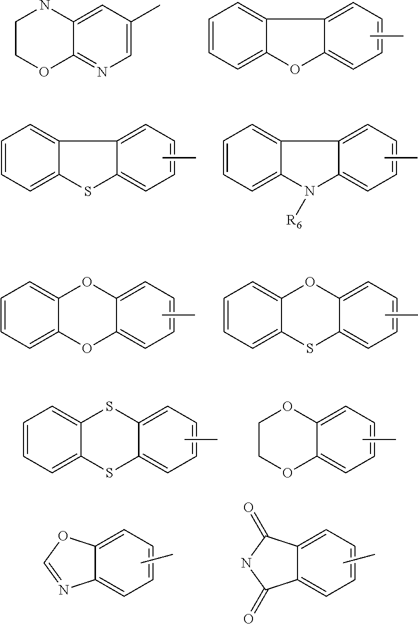

anthraquinonyl. Each of the heteroaryls may carry the substituents indicated above or in claim 1.

Phenylene is

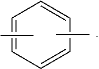

### Naphthylene is

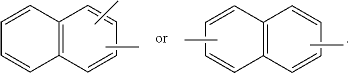

### Biphenylene is

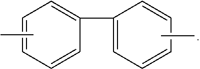

### Oxydiphenylene is

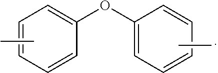

Heteroarylene is a divalent radical of the heteroaryl rings as described above, for example

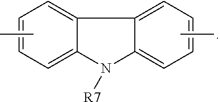

If the radicals R₂ and D₂, together with the ethylenically unsaturated double bond to which they are attached, form a 5-, 6- or 7-membered ring which optionally is interrupted by one or more O, S, NR₇ or CO, for example the following structures are obtained

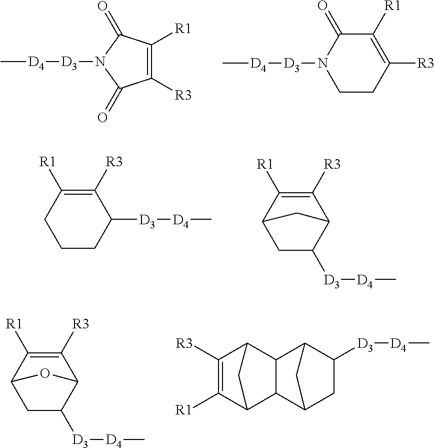

etc.

If the radicals R₃ and D₂ together with the carbon of the ethylenically unsaturated double bond to which they are attached form C₃-C₃₀cycloalkyl which optionally is interrupted by one or more O, S, NR₇ or CO, for example the following structures are obtained

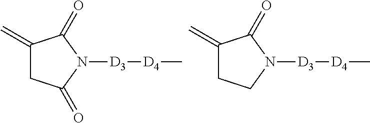

etc.

Groups having a —O—C-bond or a —O—Si-bond which cleaves upon the action of an acid, and being substituents of the radicals Ar₁, Ar₂ and Ar₃ are acid cleavable groups which increase the solubility of the compounds of formula I in the alkaline developer after reaction with an acid. This effect is for example described in U.S. Pat. No. 4,883,740.

Examples of groups suitable as such substitutents are for example known orthoesters, trityl and benzyl groups, tert.-butyl esters of carboxylic acids, tert.-butyl carbonates of phenols or silyl ethers of phenols, e.g. OSi(CH₃)₃, CH₂(CO)OC(CH₃)₃, (CO)OC(CH₃)₃, O(CO)OC(CH₃)₃ or

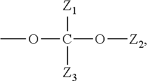

wherein Z₁ and Z₂ independently of one another are hydrogen, C₁-C₅alkyl, C₃-C₈-cycloalkyl, phenyl-C₁-C₃-alkyl, or Z₁ and Z₂ together are C₂-C₅alkylene, and
Z₃ is unsubstituted or halogen-substituted C₁-C₅alkyl, unsubstituted or halogen-substituted C₃-C₈cycloalkyl, or phenyl-C₁-C₃-alkyl, or, if Z₁ and Z₂ together are no C₂-C₅alkylene, Z₃ and Z₂ together may be C₂-C₅alkylene, which may be interrupted by O or S.

Examples of anions X⁻ as

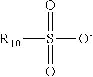

are: haloalkylSC₃⁻, e.g. C₄F₉SC₃⁻, (haloalkylSO₂)₃C⁻,

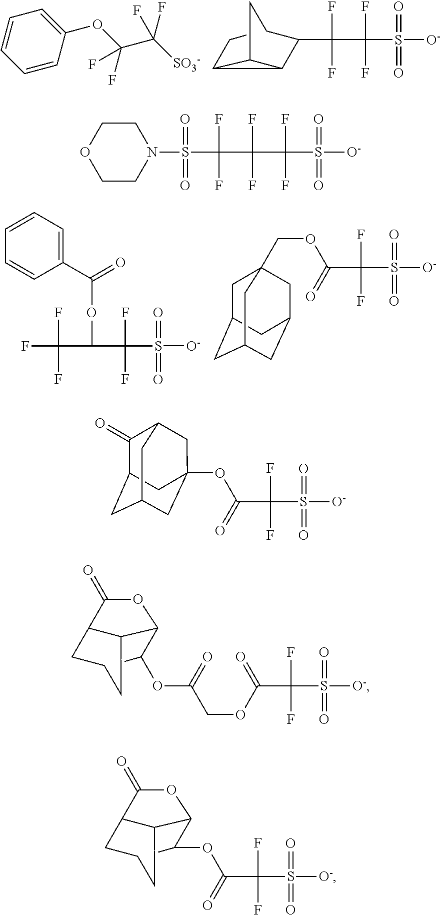

etc.

Examples of anions X⁻ as

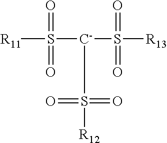

### are e.g. (CF₃—SO₂)₃C⁻, (C₄F₉—SO₂)₃C⁻, etc.

An example of anion X⁻ as

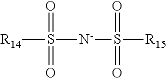

is

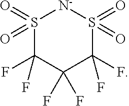

The terms “and/or” or “or/and” in the claims and throughout the specification are meant to express that not only one of the defined alternatives (substituents) may be present, but also several of the defined alternatives (substituents) together, namely mixtures of different alternatives (substituents).

The term “optionally substituted” means unsubstituted or substituted.

The term “optionally interrupted” means uninterrupted or interrupted.

“optionally” is intended to cover both corresponding options which are defined.

The term “at least” is meant to define one or more than one, for example one or two or three, preferably one or two.

The preferences referring to the compounds of the formula I as given hereinbefore and in the context of the whole text, are intended not to refer to the compounds as such only, but to all categories of the claims. That is to the compositions, comprising the compounds of the formula I, to the photoinitiator mixtures comprising said compounds, as well as the use or process claims in which said compounds are employed.

The sulfonium salts of formulae I can generally be prepared by a variety of methods described, for instance, by J. V. Crivello in Advances in Polymer Science 62, 1-48, (1984). For example, the desired sulfonium salts can be prepared by reaction of an aryl compound with sulfur monochloride in the presence of chlorine and a Lewis acid, reaction of an aryl Grignard reagent with a diaryl sulfoxide, condensation of a diaryl sulfoxide with an aryl compound in the presence of an acid, or the reaction of a diaryl sulfide with a diaryliodonium salt in the presence of a copper(II) salt. The person skilled in the art is well aware of the appropriate reactions as well as of the reaction conditions which have to be taken.

Sulfonium salts of the formula I have at least one polymerizable ethylenically unsaturated double bond. Hence polymers can be prepared employing the sulfonium salts of the formula I by methods described in the literature, for example by free radical polymerization, anionic polymerization, cationic polymerization, controlled free radical polymerization and so on.

The free radical polymerizations usually are carried out in an inert solvent such as for example water, methanol, 2-propanol, 1,4-dioxane, acetone, methyl isobutyl ketone, toluene, tetrahydrofuran (THF), propylene glycol monomethyl ether acetate (PGMEA), propylene glycol monomethyl ether (PGME), ethyl lactate (EL), or without solvent under an oxygen-free atmosphere. Peroxides such as for example dibenzoyl peroxide, diacetyl peroxide, di-t-butyl peroxalate and dicumyl peroxide; azocompounds such as for example azobis(isobutyronitrile) (AIBN), 1,1′-azobis(1-cyclohexanenitrile), 2,2-azobis(2-amidinopropane)dihydrochloride, dimethyl 2,2′-azobis(isobutyrate) and 2,2′-azobis[2-methyl-N-(2-hydroxyethyl)propionamide]; and redox systems such as for example Fe²⁺/H₂O₂ and dibenzoyl peroxide/dimethylaniline, are used as the initiator for free radical polymerization. Such reactions are well known to those skilled in the art, and generally carried out at temperature in the range of −10° C. to 150° C., preferably 40° C. to 120° C. Furthermore, for the free radical polymerization anionic surfactants, cationic surfactant or non-ionic surfactant can be added, i.e., emulsion polymerization.

The anionic polymerizations usually carried out in an inert solvent such as for example toluene, hexane, cyclohexane, tetrahydrofuran (THF), 1,4-dioxane, 1,2-dimethoxyethane, pyridine, dimethyl sulfoxide under a water- and oxygen-free atmosphere. Alkaline metals such as for example Li, Na and K; and organometallic compounds such as for example butyllithium, benzyllithium, trimethylsilylmethyllithium, phenylmagnesium bromide are used as the initiator for anionic polymerization. Such reactions are well known to those skilled in the art, and generally carried out at temperature in the range of −100° C. to 80° C., preferably −80° C. to 50° C. The cationic polymerizations usually are carried out in an inert solvent such as for example toluene, hexane, cyclohexane, dichloromethane, dioxane. Broensted acids such as for example HCl, sulfuric acid, methanesulfonic acid, trifluoromethanesulfonic acid, fluorosulfonic acid; and Lewis acids such as for example BF₃, AlCl₃, TiCl₄, SnCl₄, FeCl₃ with co-catalysts such as for example HCl, H₂O, trifluoroacetic acid, methanol are used as the initiator for cationic polymerization. Such reactions are well known to those skilled in the art, and generally carried out at temperature in the range of −100° C. to 80° C., preferably −80° C. to 50° C.

The preparation of the polymers by radical, anionic and cationic polymerizations is described in standard chemistry textbooks, for instance, G. Allen and J. C. Bevington, Comprehensive Polymer Science, Vol 3, Pergamon Press, 1989.

The polymer comprising repeating units derived from the compound of the formula I can be also synthesized by controlled free radical polymerization such as for example, nitro-oxide mediated radical polymerization (NOR) described in C. J. Hawker, A. W. Bosman, E. Harth, Chem. Rev. 101, 3661 (2001), atom transfer radical polymerization (ATRP) described in K. Matyjaszewski, J. Xia, Chem. Rev. 101, 2921 (2001), radical addition-fragmentation chain transfer mediated polymerization (RAFT) described in G. Moad, Y. K. Chong, A. Postma, E. Rizzardo, S. H. Thang, Polymer 46 8458 (2005) and so on.

Homo-polymers comprising one repeating unit derived from the compound of the formula I; and co-polymers comprising at least one repeating unit derived from the compound of the formula I and optionally repeating units derived from ethylenically unsaturated compounds selected from the group of formula II can be prepared by the polymerization methods described above.

The compounds of the formula I, and the polymers comprising repeating units derived from a compound of the formula I can be used as photosensitive acid donors.

Subject of the invention therefore is a composition comprising (b) at least one polymer comprising at least one repeating unit derived from the compound of the formula I according to claim 1 and repeating units derived from ethylenically unsaturated compounds of formula II as described above;

as well as a composition comprising
(a) a compound which cures upon the action of an acid or a compound whose solubility is increased upon the action of an acid; and
(b) at least one compound of the formula I as described above; and/or a polymer comprising at least one repeating unit derived from the compound of the formula I as described above and optionally repeating units derived from ethylenically unsaturated compounds of formula II as described above.

The compounds of the formula I, and the polymers comprising repeating units derived from a compound of the formula I can be used as photosensitive acid donors in a photoresist. They optionally also function as a compound whose solubility is increased upon the action of an acid, that is as part of component (a) as defined above. Resist systems can be prepared by image-wise irradiation of systems comprising compounds of formula I and/or a polymer comprising repeating units derived from a compound of the formula I followed by a developing step.

The invention accordingly relates to a chemically amplified photoresist composition comprising
(a) a compound which cures upon the action of an acid; or a compound whose solubility is increased upon the action of an acid; and/or
(b) at least one compound of the formula I according to claim 1; and/or a polymer as described above.

In general,

(i) the composition according to the present invention comprises as component (a) a compound which cures upon the action of an acid; or a compound whose solubility is increased upon the action of an acid and, as component (b), a photolatent acid generator compound of the formula I; or
(ii) the composition comprises component (a) as described above and a polymer prepared by polymerizing or copolymerizing a compound of the formula I, not containing an acid-labile group; or
(iii) the composition comprises component (a) as described above and a polymer prepared by polymerizing or copolymerizing a compound of the formula I, containing an acid-labile group; or
(iv) the composition comprises a polymer prepared by polymerizing or copolymerizing a compound of the formula I, containing an acid-labile group.

In the latter case (iv), the polymer constitutes both, component (a) and component (b) as well.

A chemically amplified photoresist is understood to be a resist composition wherein the radiation sensitive component provides a catalytic amount of acid which subsequently catalyses a chemical reaction of at least one acid-sensitive component of the resist. Resulting is the induction of a solubility difference between the irradiated and non-irradiated areas of the resist. Because of the catalytic nature of this process one acid molecule can trigger reactions at multiple sites as it diffuses through the reactive polymer matrix, from one reaction site to the next, as long as it is not trapped or destroyed by any secondary reaction. Therefore, a small acid concentration is sufficient to induce a high difference in the solubility between exposed and unexposed areas in the resist. Thus, only a small concentration of the latent acid compound is necessary. As a result, resists with high contrast and high transparency at the exposure wavelength in optical imaging can be formulated, which in turn produce steep, vertical image profiles at high photosensitivity. However, as a result of this catalytic process, it is required that the latent acid catalysts are chemically and thermally very stable (as long as not irradiated) in order not to generate acid during resist storage or during processing, which—in most cases—requires a post exposure bake step to start or to complete the catalytic reaction which leads to the solubility differential. It is also required to have good solubility of the latent catalysts in the liquid resist formulation and the solid resist film to avoid any particle generation which would interfere with the application of these resists in microelectronic manufacturing processes.

In contrast, positive resist materials which are not based on the chemical amplification mechanism must contain a high concentration of the latent acid, because it is only the acid concentration which is generated from the latent acid under exposure which contributes to the increased solubility of the exposed areas in alkaline developer. Because small acid concentration has only a little effect on the change of the dissolution rate of such resist and the reaction proceeds typically without a post exposure bake here, the requirements regarding chemical and thermal stability of the latent acid are less demanding than for chemically amplified positive resists. These resists require also a much higher exposure dose to generate enough acid for achieving sufficient solubility in the alkaline developer in the exposed areas and also suffer from the relatively low optical transparency (due to the high concentration of latent acid necessary) and thus also lower resolution and sloped images. Resist compositions based on non-chemically amplified technology are therefore inferior in photosensitivity, resolution and image quality compared to chemically amplified resists.

From the above it becomes clear that chemical and thermal stability of a latent catalyst is vital for a chemically amplified resist and that latent acids which can work in a non-chemically amplified resist are not necessarily applicable to chemically amplified resists because of the different acid diffusion requirements, acid strength requirements and thermal and chemical stability requirements.

The difference in resist solubility between irradiated and non-irradiated sections that occurs as a result of the acid-catalysed reaction of the resist material during or after irradiation of the resist may be of two types depending upon which further constituents are present in the resist. If the compositions according to the invention comprise components that increase the solubility of the composition in the developer after irradiation, the resist is positive.

The invention accordingly relates to a chemically amplified photoresist composition, which is a positive resist.

If, on the other hand, the components of the formulation reduce the solubility of the composition after irradiation, the resist is negative.

The invention accordingly relates also to a chemically amplified photoresist composition, which is a negative photoresist.

Interesting is a chemically amplified positive photoresist composition as describe above, as component (b) comprising a polymer, comprising repeating units derived from a compound of the formula I as described above.

In particular preferred is a chemically amplified positive photoresist composition, wherein component (b) is at least one polymer comprising at least one repeating unit derived from a compound of the formula I according to claim 1; and

at least one repeating unit derived from ethylenically unsaturated of formula II,

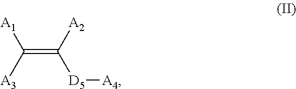

wherein
A₁, A₂, A₃, A₄ and D₅ are as defined above.

A monomeric or polymeric compound which—in the unexposed areas—reduces the dissolution rate of an additionally present alkaline soluble binder resin in the resist formulation and which is essentially alkali-insoluble in the unexposed areas so that the resist film remains in the unexposed area after development in alkaline solution, but which is cleaved in the presence of acid, or is capable of being rearranged, in such a manner that its reaction product becomes soluble in the alkaline developer is referred to hereinafter as dissolution inhibitor.

The invention includes, as a special embodiment a chemically amplified positive alkaline-developable photoresist composition, comprising

(a1) at least one polymer having acid-labile groups which decompose in the presence of an acid and increase the solubility of the resist film in an aqueous alkaline developer solution in the exposed area and/or
(b) at least one compound of formula I, and/or a polymer comprising repeating units derived from a compound of the formula I.

A further embodiment of the invention is a chemically amplified positive alkaline-developable photoresist composition, comprising

(a2) at least one monomeric or oligomeric dissolution inhibitor having at least one acid-labile group which decomposes in the presence of acid and increases the solubility in an aqueous alkaline developer solution and at least one alkali-soluble polymer and/or,
(b) at least one compound of formula I and/or a polymer comprising repeating units derived from a compound of the formula I.

Another specific embodiment of the invention resides in a chemically amplified positive alkaline-developable photoresist composition, comprising

(a1) at least one polymer having acid labile groups which decompose in the presence of an acid and increase the solubility in an alkaline developer in the exposed area;
(a2) at least one a monomeric or oligomeric dissolution inhibitor, having at least one acid labile group, which decomposes in the presence of an acid and increase the alkaline solubility in the exposed area;
(a3) at least one an alkali-soluble monomeric, oligomeric or polymeric compound at a concentration which still keeps the resist film in the unexposed area essentially insoluble in the alkaline developer, and/or
(b) at least one compound of formula I and/or a polymer comprising repeating units derived from a compound of the formula I.

The invention therefore pertains to a chemically amplified photoresist composition, comprising

(a1)) at least one polymer having an acid-labile group which decomposes in the presence of an acid to increase the solubility in aqueous alkaline developer solution and/or
(a2) at least one monomeric or oligomeric dissolution inhibitor having an acid-labile group which decomposes in the presence of an acid to increase the solubility in aqueous alkaline developer solution and/or
(a3) at least one alkali-soluble monomeric, oligomeric or polymeric compound; and
(b) as photosensitive acid donor, at least one compound of formula I and/or a polymer comprising repeating units derived from a compound of the formula I.

Preferably the composition is a chemically amplified positive photoresist composition, comprising
(al) at least one polymer having an acid-labile group which decomposes in the presence of an acid to increase the solubility in aqueous alkaline developer solution; and/or
(a2) at least one monomeric or oligomeric dissolution inhibitor having an acid-labile group which decomposes in the presence of an acid to increase the solubility in aqueous alkaline developer solution; and/or
(a3) at least one alkali-soluble monomeric, oligomeric or polymeric compound; and
(b) at least one compound of the formula I; and/or a polymer comprising at least one repeating unit derived from the compound of the formula I and at least one repeating unit derived from ethylenically unsaturated compounds of formula II as described above.

The compositions may comprise additionally to the component (b) other photosensitive acid donors and/or (c) other additives.

A polymer having acid labile groups which decompose in the presence of an acid and increase the solubility in an alkaline developer can comprise photosensitive acid donor groups in the polymer. Such polymer can simultaneously work as photosensitive acid donor and as polymer whose solubility is increased upon the action of an acid in a chemically amplified positive photoresist composition.

The present invention pertains to a chemically amplified positive photoresist composition, comprising

(b) as photosensitive acid donor and as compound whose solubility is increased upon the action of an acid, at least one compound of the formula I; and/or a polymer comprising at least one repeating unit derived from a compound of the formula I and repeating units derived from ethylenically unsaturated compounds of formula II as described above.

Chemically amplified positive resist systems are described, for example, in E. Reichmanis, F. M. Houlihan, O. Nalamasu, T. X. Neenan, Chem. Mater. 1991, 3, 394; or in C. G. Willson, “Introduction to Microlithography, 2nd. Ed.; L. S. Thompson, C. G. Willson, M. J. Bowden, Eds., Amer. Chem. Soc., Washington D.C., 1994, p. 139.

Suitable examples of acid-labile groups which decompose in the presence of an acid to produce aromatic hydroxy groups, carboxylic groups, keto groups and aldehyde groups and increase the solubility in aqueous alkaline developer solution are, for example, alkoxyalkyl ether groups, tetrahydrofuranyl ether groups, tetrahydropyranyl ether groups, tert.-alkyl ester groups, trityl ether groups, silyl ether groups, alkyl carbonate groups as for example tert.-butyloxycarbonyloxy-, trityl ester groups, silyl ester groups, alkoxymethyl ester groups, cumyl ester groups, acetal groups, ketal groups, tetrahydropyranyl ester groups, tetrafuranyl ester groups, tertiary alkyl ether groups, tertiary alkyl ester groups, and the like. Examples of such group include alkyl esters such as methyl ester and tert-butyl ester, acetal type esters such as methoxymethyl ester, ethoxymethyl enter, 1-ethoxyethyl ester, 1-isobutoxyethyl ester, 1-iso-propoxyethyl ester, 1-ethoxypropyl ester, 1-(2-methoxyethoxy)ethyl ester, 1-(2-acetoxyethoxy)ethyl ester, 1-[2-(1-adamantyloxy)ethoxy]ethyl ester, 1-[2-(1-adamantylcarbonyloxy)ethoxy]ethyl ester, tetrahydro-2-furyl ester and tetrahydro-2-pyranyl ester, and alicyclic ester such as isobornyl ester.

The polymer having functional groups capable of decomposing by the action of an acid to enhance solubility of the resist film comprising this polymer in an alkaline developing solution, which can be incorporated in the positive resist according to the present invention, may have the acid-labile groups in the backbone and/or side chains thereof, preferably in side chains thereof.

The polymer having acid-labile groups suitable for the use in the present invention can be obtained with a polymer analogous reaction where the alkaline soluble groups are partially or completely converted into the respective acid labile groups or directly by (co)-polymerization of monomers which have the acid labile groups already attached, as is for instance disclosed in EP 254853, EP 878738, EP 877293, JP-A-2-25850, JP-A-3-223860, and JP-A-4-251259.

The polymers which have acid labile groups pendant to the polymer backbone, in the present invention preferably are polymers which have, for example silylether, acetal, ketal and alkoxyalkylester groups (called “low-activation energy blocking groups”) which cleave completely at relatively low post exposure bake temperatures (typically between room temperature and 110° C.) and polymers which have, for example, tert-butylester groups or tert.-butyloxycarbonyl (TBOC) groups or other ester groups which contain a secondary or tertiary carbon atom next to the oxygen atom of the ester bond (called “high-activation energy blocking groups”) which need higher bake temperatures (typically >110° C.) in order to complete the deblocking reaction in the presence of acid. Hybrid systems can also be applied, wherein, both, high activation energy blocking groups as well as low activation energy blocking groups are present within one polymer. Alternatively, polymer blends of polymers, each utilizing a different blocking group chemistry, can be used in the photosensitive positive resist compositions according to the invention.

Preferred polymers which have acid labile groups are polymers and co-polymers comprising the following distinct monomer types:

1) monomers that contain acid-labile groups which decompose in the presence of an acid to increase the solubility in aqueous alkaline developer solution and
2) monomers that are free of acid labile groups and free of groups that contribute to the alkaline solubility and/or
3) monomers that contribute to aqueous alkaline solubility of the polymer.

Examples of monomers of type 1) are:

non-cyclic or cyclic secondary and tertiary-alkyl (meth)acrylates such as butyl acrylate, including t-butyl acrylate, butyl methacrylate, including t-butyl methacrylate, 3-oxocyclohexyl (meth)acrylate, tetrahydropyranyl (meth)acrylate, 2-methyl-adamantyl (meth)acrylate, cyclohexyl (meth)acrylate, norbornyl (meth)acrylate, (2-tetrahydropyranyl)oxynorbonylalcohol acrylates, (2-tetrahydropyranyl)oxymethyltricyclododecanemethanol methacrylates, trimethylsilylmethyl (meth)acrylate, (2-tetrahydropyranyl)oxynorbonylalcohol acrylates, (2-tetrahydropyranyl)oxymethyltricyclododecanemethanol methacrylates, trimethylsilylmethyl (meth)acrylate o-/m-/p- (3-oxocyclohexyloxy)styrene, o-/m-/p- (1-methyl-1-phenylethoxy)styrene, o-/m-/p- tetrahydropyranyloxystyrene, o-/m-/p- adamantyloxystyrene, o-/m-/p- cyclohexyloxystyrene, o-/m-/p- norbornyloxystyrene, non-cyclic or cyclic alkoxycarbonylstyrenes such as o-/m-/p- butoxycarbonylstyrene, including p- t-butoxycarbonylstyrene, o-/m-/p- (3-oxocyclohexyloxycarbonyl)-styrene, o-/m-/p- (1-methyl-1-phenylethoxycarbonyl)styrene, o-/m-/p- tetrahydropyranyloxycarbonylstyrene, o-/m-/p- adamantyloxycarbonylstyrene, o-/m-/p- cyclohexyloxycarbonylsyrene, o-/m-/p- norbornyloxycarbonylstyrene, non-cyclic or cyclic alkoxycarbonyloxystyrenes such as o-/m-/p- butoxycarbonyloxystyrene, including p- t-butoxycarbonyloxystyrene, o-/m-/p- (3-oxocyclohexyloxycarbonyloxy)styrene, o-/m-/p- (1-methyl-1-phenylethoxycarbonyloxy)styrene, o-/m-/p- tetrahydropyranyloxycarbonyloxystyrene, o-/m-/p- adamantyloxycarbonyloxystyrene, o-/m-/p- cyclohexyloxycarbonyloxystyrene, o-/m-/p- norbornyloxycarbonyloxystyrene, non-cyclic or cyclic alkoxycarbonylalkoxystyrenes such aso/m/p- butoxycarbonylmethoxystyrene, p- t-butoxycarbonylmethoxystyrene, o-/m-/p- (3-oxocyclohexyloxycarbonylmethoxy)styrene, o-/m-/p- (1-methyl-1-phenylethoxycarbonylmethoxy)styrene, o-/m-/p- tetra-hydropyranyloxycarbonylmethoxystyrene, o-/m-/p- adamantyloxycarbonylmethoxystyrene, o-/m-/p- cyclohexyloxycarbonylmethoxystyrene, o-/m-/p- norbornyloxycarbonylmethoxystyrene, trimethylsiloxystyrene, dimethyl(butyl)siloxystyrene, unsaturated alkyl acetates such as isopropenyl acetate and the derivatives of thereof.

Monomers of type 1) bearing low activation energy acid labile groups include, for example, p- or m-(1-methoxy-1-methylethoxy)-styrene, p- or m-(1-methoxy-1-methylethoxy)-methylstyrene, p- or m-(1-methoxy-1-methylpropoxy)styrene, p- or m-(1-methoxy-1-methylpropoxy) methylstyrene, p- or m-(1-methoxyethoxy)-styrene, p- or m-(1-methoxyethoxy)-methylstyrene, p- or m-(1-ethoxy-1-methylethoxy)styrene, p- or m-(1-ethoxy-1-methylethoxy)-methylstyrene, p- or m-(1-ethoxy-1-methylpropoxy)styrene, p- or m-(1-ethoxy-1-methylpropoxy)-methylstyrene, p- or m-(1-ethoxyethoxy)styrene, p- or m-(1-ethoxyethoxy)-methylstyrene, p- (1-ethoxyphenyl-ethoxy)styrene, p- or m-(1-n-propoxy-1-methylethoxy)styrene, p- or m-(1-n-propoxy-1-methylethoxy)-methylstyrene, p- or m-(1-n-propoxyethoxy)styrene, p- or m-(1-n-propoxyethoxy)-methylstyrene, p- or m-(1-isopropoxy-1-methylethoxy)styrene, p- or m-(1-isopropoxy-1-methylethoxy)-methylstyrene, p- or m-(1-isopropoxyethoxy)styrene, p- or m-(1-isopropoxyethoxy)-methylstyrene, p- or m-(1-isopropoxy-1-methylpropoxy)styrene, p- or m-(1-isopropoxy-1-methylporpoxy)-methylstyrene, p- or m-(1-isopropoxypropoxy)styrene, p- or m-(1-isopropoxypropoxy)-methylstyrene, p- or m-(1-n-butoxy-1-methylethoxy)styrene, p- or m-(1-n-butoxyethoxy)styrene, p- or m-(1-isobutoxy-1-methylethoxy)styrene, p- or m-(1-tert-butoxy-1-methylethoxy)styrene, p- or m-(1-n-pentoxy-1-methylethoxy)styrene, p- or m-(1-isoamyloxy-1-methylethoxy)styrene, p- or m-(1-n-hexyloxy-1-methylethoxy)styrene, p- or m-(1-cyclohexyloxy-1-methylethoxy)styrene, p- or m-(1-trimethylsilyloxy-1-methylethoxy)-styrene, p- or m-(1-trimethylsilyloxy-1-methylethoxy)-methylstyrene, p- or m-(1-benzyloxy-1-methylethoxy)styrene, p- or m-(1-benzyloxy-1-methylethoxy)-methylstyrene, p- or m-(1-methoxy-1-methylethoxy)styrene, p- or m-(1-methoxy-1-methylethoxy)-methylstyrene, p- or m-(1-trimethylsilyloxy-1-methylethoxy)styrene p- or m-(1-trimethylsilyloxy-1-methylethoxy)-methylstyrene. Other examples of polymers having alkoxyalkylester acid labile groups are given in U.S. Pat. No. 5,225,316 and EP 829766. Examples of polymers with acetal blocking groups are given in U.S. Pat. No. 5,670,299, EP 780732, U.S. Pat. No. 5,627,006, U.S. Pat. No. 5,558,976, U.S. Pat. No. 5,558,971, U.S. Pat. No. 5,468,589, EP 704762, EP 762206, EP 342498, EP 553737 and described in ACS Symp. Ser. 614, Microelectronics Technology, pp. 35-55 (1995) and J. Photopolymer Sci. Technol. Vol. 10, No. 4 (1997), pp. 571-578. The polymer used in the present invention is not limited thereto.

With respect to polymers having acetal groups as acid-labile groups, it is possible to incorporate acid labile crosslinks as for example described in H.-T. Schacht, P. Falcigno, N. Muenzel, R. Schulz, and A. Medina, ACS Symp. Ser. 706 (Micro- and Nanopatterning Polymers), p. 78-94, 1997; H.-T. Schacht, N. Muenzel, P. Falcigno, H. Holzwarth, and J. Schneider, J. Photopolymer Science and Technology, Vol. 9, (1996), 573-586. This crosslinked system is preferred from the standpoint of heat resistance of the resist patterns.

Monomers with high activation energy acid labile groups are, for example, p-tert.-butoxycarbonyloxystyrene, tert.-butyl-acrylate, tert.-butyl-methacrylate, 2-methyl-2-adamantyl-methacrylate, isobornyl-methacrylate.

Monomers of type 1) suitable for ArF resist technology in particular include, for example, 2-methyl-2-adamantyl acrylate, 2-ethyl-2-adamantyl acrylate, 2-n-butyl-2-adamantyl acrylate, 2-n-butyl-2-adamantyl methacrylate, 2-methyl-2-adamantyl methacrylate, 2-ethyl-2-adamantyl methacrylate2-(1-adamantyl)isopropyl methacrylate, 2-(1-adamantyl)isopropyl acrylate, 2-(1-adamantyl)isobutyl methacrylate, 2-(1-adamantyl) isobutyl acrylate, t-butyl methacrylate, t-butyl acrylate, 1-methylcyclohexyl methacrylate, 1-methylcyclohexyl acrylate, 1-ethylcyclohexyl methacrylate, 1-ethylcyclohexyl acrylate, 1-(n-propyl)cyclohexyl methacrylate, 1-(n-propyl)cyclohexyl acrylate, tetrahydro-2-methacryloyloxy-2H-pyran and tetrahydro-2-acryloyloxy-2H-pyran. Other monomers comprising acid-labile adamantyl moieties are disclosed in JP-A-2002-1265530, JP-A-2002-338627, JP-A-2002-169290, JP-A-2002-241442, JP-A-2002-145954, JP-A-2002-275215, JP-A-2002-156750, JP-A-2002-268222, JP-A-2002-169292, JP-A-2002-162745, JP-A-2002-301161, WO02/06901 A2, JP-A-2002-311590, JP-A-2002-182393, JP-A-2002-371114, JP-A-2002-162745.

Particular olefins with acid labile-group are also suitable for ArF resist technology as shown in, for example, JP-A-2002-308938, JP-A-2002-308869, JP-A-2002-206009, JP-A-2002-179624, JP-A-2002-161116.

Examples of comonomers according to type 2) are:

aromatic vinyl monomers, such as styrene, α-methylstyrene, acetoxystyrene, α-methylnaphthylene, acenaphthylene, vinyl alicyclic compounds such as vinyl norbornane, vinyl adamantine. vinyl cyclohexane, alkyl (meth)acrylates such as methyl methacrylate, (meth)-acrylonitrile, vinylcyclohexane, vinylcyclohexanol, itaconic anhydride, as well as maleic anhydride.

Comonomers according to type 2) suitable for ArF resist technology in particular include, for example, alpha-acryloyloxy-gamma-butyrolactone, alpha-methacryloyloxy-gamma-butyrolactone, alpha-acryloyloxy-beta,beta-dimethyl-gamma-butyro-lactone, alpha-methacryloyloxy-beta,beta-dimethyl-gamma-butyrolactone, alpha-acryloyloxy-alpha-methyl-gamma-butyrolactone, alpha-methacryloyloxy-alpha-methyl-gamma-butyrolactone, beta-acryloyloxy-gamma,beta-methacryloyloxy-alpha-methyl-gamma-butyrolactone, 5-acryloyloxy-2,6-norbornanecarbolactone, 5-methacryloyloxy-2,6-norbolnanecarbolactone, 2-norbornene, methyl 5-norbornene-2-carboxylate, tert-butyl 5-norbornene-2-carboxylate, 1-cyclohexyl-1-methylethyl 5-norbornene-2-carboxylate, 1-(4-methylcyclohexyl)-1-methylethyl 5-norbornene-2-carboxylate, 1-methyl-1-(4-oxocyclohexyl)ethyl 5-norbornene-2-carboxylate, 1-(1-adamatyl)-1-methylethyl 5-norbornene-2-carboxylate,1-methylcyclohexyl 5-norbornene-2-carboxylate, 2-methyl-2-adamantyl 5-norbornene-2-carboxylate, 2-ethyl-2-adamantyl 5-norbornene-2-carboxylate, 5-norbornene-2,3-dicarboxylic acid anhydrate, 2(5H)-furanone, 3-vinyl-gamma-butyrolactone, 3-methacryloyloxybicyclo[4,3,0]nonane, 3-acryloyloxybicyclo[4,3,0]nonane, 1-adamantyl methacrylate, 1-adamantyl acrylate, 3-methacryloyloxymethyltetracyclo[4,4,0,1^{2,5},1^{7,10}]dodecane, 3-acryloyloxymethyltetracyclo[4,4,0,1^{2,5},1^{7,10}]dodecane, 2-methacryloyloxynorbornane, 2-acryloyloxynorbornane, 2-methacryloyloxyisobornane, 2-acryloyloxyisobornane, 2-methacryloyloxymethylnorbornane, 2-acryloyloxymethylnorbornane.

Examples of comonomers according to type 3) are:

vinyl aromatic compounds such as hydroxystyrene, acrylic acid compounds such as methacrylic acid, ethylcarbonyloxystyrene and derivatives of thereof. These polymers are described, for example, in U.S. Pat. No. 5,827,634, U.S. Pat. No. 5,625,020, U.S. Pat. No. 5,492,793, U.S. Pat. No. 5,372,912, EP 660187, U.S. Pat. No. 5,679,495, EP 813113 and EP 831369. Further examples are crotonic acid, isocrotonic acid, 3-butenoic acid, acrylic acid, 4-pentenoic acid, propiolic acid, 2-butynoic acid, maleic acid, fumaric acid, and acetylenecarboxylic acid. The polymer used in the present invention is not limited thereto.

Comonomers according to type 3) suitable for ArF resist technology in particular include, for example, 3-hydroxy-1-adamantyl acrylate, 3-hydroxy-1-adamantyl methacrylate, 3,5-dihydroxy-1-adamantyl acrylate, 3,5-dihydroxy-1-adamantyl methacrylate, 2-hydroxy-5-norbornene, 5-norbornene-2-carboxylic acid, 1-(4-hydroxycyclohexyl)-1-methylethyl 5-norbornene-2-carboxylate, 2-hydroxy-1-ethyl 5-norbornene-2-carboxylate, 5-norbornene-2-methanol, 8-hydroxymethyl-4-methacryloyloxymethyltricyclo[5.2.1.0^{2.6}]decane, 8-hydroxymethyl-4-acryloyloxymethyltricyclo[5.2.1.0^{2.6}]decane, 4-hydroxymethyl-8-methacryloyloxymethyltricyclo[5.2.1.0^{2.6}]decane, 4-hydroxymethyl-8-acryloyloxymethyltricyclo[5.2.1.0²⁶]decane.

Other monomers comprising lactone moieties suitable for ArF technology are disclosed in, for example, JP-A-2002-6502, JP-A-2002-145955, EP1127870A1, JP-A-2002-357905, JP-A-2002-296783. Other olefins suitable for ArF technology are published in, for example, JP-A-2002-351078, JP-A-2002-234918, JP-A-2002-251009, EP 1127870A1, JP-A-2002-328475, JP-A-2002-278069, JP-A-2003-43689, JP-A-2002-202604, WO01/86353, JP-A-2002-23371, JP-A-2002-72484, JP-A-2002-202604, JP-A-2001-330959, JP-A-2002-3537, JP-A-2002-30114, JP-A-2002-278071, JP-A-2002-251011, JP-A-2003-122010, JP-A-2002-139837, JP-A-2003-195504, JP-A-2001-264984, JP-A-2002-278069, JP-A-2002-328475, U.S. Pat. No. 6,379,861, U.S. Pat. No. 6,599,677, US2002/119391, U.S. Pat. No. 6,277,538, US2003/78354.

The content of acid labile monomers in the polymer may vary over a wide range and depends on the amount of the other comonomers and the alkaline solubility of the deprotected polymer. Typically, the content of monomers with acid labile groups in the polymer is between 5 and 60 mol %. If the content is too small, too low development rates and residues of the resist in the exposed areas result. If the content of acid labile monomers is too high, resist patterns are poorly defined (eroded) after development and narrow features cannot be resolved anymore and/or the resist looses its adhesion to the substrate during development. Preferably the copolymers which have acid labile groups have a M_{W} of from about 3′000 to about 200′000, more preferably from about 5′000 to about 50′000 with a molecular weight distribution of about 3 or less, more preferably a molecular weight distribution of about 2 or less. Non-phenolic polymers, e.g. a copolymer of an alkyl acrylate such as t-butyl acrylate or t-butyl-methacrylate and a vinyl alicyclic compound, such as a vinyl norbornanyl or vinyl cyclohexanol compound, also may be prepared by such free radical polymerization or other known procedures and suitably will have a M_{W} of from about 8′000 to about 50′000, and a molecular weight distribution of about 3 or less.

Other comonomers may suitably be added in an appropriate amount for the purpose of controlling the glass transition point of the polymer and the like.

In the present invention a mixture of two or more polymers having acid-labile groups may be used. For example, use may be made of a mixture of a polymer having acid-labile groups, which are cleaved very easily, such as acetal groups or tetrahydropyranyloxy-groups and a polymer having acid-cleavable groups, that are less easily cleaved, such as for example tertiary alkyl ester groups. Also, acid cleavable groups of different size can be combined by blending two or more polymers having different acid cleavable groups, such as a tert-butylester group and 2-methyl-adamantyl group or an 1-ethoxy-ethoxy group and a tetrahydropyranyloxy group. A mixture of a non-crosslinked resin and a crosslinked resin may also be used. The amount of these polymers in the present invention is preferably from 30 to 99% by weight, more preferably from 50 to 98% by weight, based on the total amount of all solid components. An alkali-soluble resin or monomeric or oligomeric compound having no acid-labile groups may be further incorporated into the composition in order to control the alkali solubility.

Examples of polymer blends with polymers having different acid-labile groups are given in EP 780732, EP 679951 and U.S. Pat. No. 5,817,444.

Preferably monomeric and oligomeric dissolution inhibitors (a2) are used in the present invention.

The monomeric or oligomeric dissolution inhibitor having the acid-labile group for use in the present invention is a compound which has at least one acid-labile group in the molecular structure, which decomposes in the presence of acid to increase the solubility in aqueous alkaline developer solution. Examples are alkoxymethyl ether groups, tetrahydrofuranyl ether groups, tetrahydropyranyl ether groups, alkoxyethyl ether groups, trityl ether groups, silyl ether groups, alkyl carbonate groups, trityl ester groups, silyl ester groups, alkoxymethyl ester groups, vinyl carbamate groups, tertiary alkyl carbamate groups, trityl amino groups, cumyl ester groups, acetal groups, ketal groups, tetrahydropyranyl ester groups, tetrafuranyl ester groups, tertiary alkyl ether groups, tertiary alkyl ester groups, and the like. The molecular weight of the acid-decomposable dissolution inhibitive compound for use in the present invention is 3′000 or lower, preferably from 100 to 3′000, more preferably from 200 to 2′500.

Examples of monomeric and oligomeric dissolution inhibitors having acid-labile groups are described as formulae (I) to (XVI) in EP 0831369. Other suitable dissolution inhibitors having acid-labile groups are shown in U.S. Pat. No. 5,356,752, U.S. Pat. No. 5,037,721, U.S. Pat. No. 5,015,554, JP-A-1-289946, JP-A-1-289947, JP-A-2-2560, JP-A-3-128959, JP-A-3-158855, JP-A-3-179353, JP-A-3-191351, JP-A-3-200251, JP-A-3-200252, JP-A-3-200253, JP-A-3-200254, JP-A-3-200255, JP-A-3-259149, JA-3-279958, JP-A-3-279959, JP-A-4-1650, JP-A-4-1651, JP-A-11260, JP-A-4-12356, JP-A-4-123567, JP-A-1-289946, JP-A-3-128959, JP-A-3-158855, JP-A-3-179353, JP-A-3-191351, JP-A-3-200251, JP-A-3-200252, JP-A-3-200253, JP-A-3-200254, JP-A-3-200255, JP-A-3-259149, JP-A-3-279958, JP-A-3-279959, JP-A-4-1650, JP-A-4-1651, JP-A-11260, JP-A-4-12356, JP-A-4-12357 and Japanese Patent Applications Nos. 3-33229, 3-230790, 3-320438, 4-254157, 4-52732, 4-103215, 4-104542, 4-107885, 4-107889, 4-152195, 4-254157, 4-103215, 4-104542, 4-107885, 4-107889, and 4-152195.

The composition can also contain polymeric dissolution inhibitors, for example, polyacetals as described for example in U.S. Pat. No. 5,354,643 or poly-N,O-acetals for example those described in U.S. Pat. No. 5,498,506, either in combination with an alkaline soluble polymer, or in combination with a polymer containing acid labile groups which increase the solubility of the resist film in the developer after exposure, or with a combination of both types of polymers.

In the case where the dissolution inhibitor having acid-labile groups is used in the present invention in combination with the compounds of formula I, the alkali-soluble polymer and/or the polymer having acid-labile groups, the amount of the dissolution inhibitor is from 3 to 55% by weight, preferably from 5 to 45% by weight, most preferably from 10 to 35% by weight, based on the total amount of all solid components of the photosensitive composition.

A polymer soluble in an aqueous alkali solution (a3) is preferably used in the present invention. Examples of these polymers include novolak resins, hydrogenated novolak resins, acetone-pyrogallol resins, poly(o-hydroxystyrene), poly(m-hydroxystyrene), poly(p-hydroxystyrene), hydrogenated poly(hydroxystyrene)s, halogen- or alkyl-substituted poly(hydroxystyrene)s, hydroxystyrene/N-substituted maleimide copolymers, o/p- and m/p-hydroxystyrene copolymers, partially o-alkylated poly(hydroxystyrene)s, [e.g., o-methylated, o-(1-methoxy)ethylated, o-(1-ethoxy)ethylated, o-2-tetrahydropyranylated, and o-(t-butoxycarbonyl)methylated poly(hydroxystyrene)s having a degree of substitution of from 5 to 30 mol % of the hydroxyl groups], o-acylated poly(hydroxystyrene)s [e.g., o-acetylated and o-(t-butoxy)carbonylated poly(hydroxystyrene)s having a degree of substitution of from 5 to 30 mol % of the hydroxyl groups], styrene/maleic anhydride copolymers, styrene/hydroxystyrene copolymers, α-methylstyrene/hydroxystyrene copolymers, carboxylated methacrylic resins, and derivatives thereof. Further suitable are poly (meth)acrylic acid [e.g. poly(acrylic acid)], (meth)acrylic acid/(meth)acrylate copolymers [e.g. acrylic acid/methyl acrylate copolymers, methacrylic acid/methyl methacrylate copolymers or methacrylic acid/methyl methacrylate/t-butyl methacrylate copolymers], (meth)acrylic acid/alkene copolymers [e.g. acrylic acid/ethylene copolymers], (meth)acrylic acid/(meth)acrylamide copolymers [e.g. acrylic acid/acrylamide copolymers], (meth)acrylic acid/vinyl chloride copolymers [e.g. acrylic acid/vinyl chloride copolymers], (meth)acrylic acid/vinyl acetate copolymer [e.g. acrylic acid/vinyl acetate copolymers], maleic acid/vinyl ether copolymers [e.g. maleic acid/methyl vinyl ether copolymers], maleic acid mono ester/methyl vinyl ester copolymers [e.g. maleic acid mono methyl ester/methyl vinyl ether copolymers], maleic acid/(meth)acrylic acid copolymers [e.g. maleic acid/acrylic acid copolymers or maleic acid/methacrylic acid copolymers], maleic acid/(meth)acrylate copolymers [e.g. maleic acid/methyl acrylate copolymers], maleic acid/-vinyl chloride copolymers, maleic acid/vinyl acetate copolymers and maleic acid/alkene copolymers [e.g. maleic acid/ethylene copolymers and maleic acid/1-chloropropene copolymers]. However, the alkali-soluble polymer for use in the present invention should not be construed as being limited to these examples.

Especially preferred alkali-soluble polymers (a3) are novolak resins, poly(o-hydroxystyrene), poly(m-hydroxystyrene), poly(p-hydroxystyrene), copolymers of the respective hydroxystyrene monomers, for example with p-vinylcyclohexanol, alkyl-substituted poly(hydroxystyrene)s, partially o- or m-alkylated and o- or m-acylated poly(hydroxystyrene)s, styrene/hydroxystyrene copolymer, and α-methylstyrene/hydroxystyrene copolymers. The novolak resins are obtained by addition-condensing one or more given monomers as the main ingredient with one or more aldehydes in the presence of an acid catalyst.

Examples of monomers useful in preparing alkaline soluble resins include hydroxylated aromatic compounds such as phenol, cresols, i.e., m-cresol, p-cresol, and o-cresol, xylenols, e.g., 2,5-xylenol, 3,5-xylenol, 3,4-xylenol, and 2,3-xylenol, alkoxyphenols, e.g., p-methoxyphenol, m-methoxyphenol, 3,5-dimethoxyphenol, 2-methoxy-4-methylphenol, m-ethoxyphenol, p-ethoxyphenol, m-propoxyphenol, p-propoxyphenol, m-butoxyphenol, and p-butoxyphenol, dialkylphenols, e.g., 2-methyl-4-isopropylphenol, and other hydroxylated aromatics including m-chlorophenol, p-chlorophenol, o-chlorophenol, dihydroxybiphenyl, bisphenol A, phenylphenol, resorcinol, and naphthol. These compounds may be used alone or as a mixture of two or more thereof. The main monomers for novolak resins should not be construed as being limited to the above examples.

Examples of the aldehydes for polycondensation with phenolic compounds to obtain novolaks include formaldehyde, p-formaldehyde, acetaldehyde, propionaldehyde, benzaldehyde, phenylacetaldehyde, α-phenylpropionaldehyde, β-phenylpropionaldehyde, o-hydroxybenzaldehyde, m-hydroxybenzaldehyde, p-hydroxybenzaldehyde, o-chlorobenzaldehyde, m-chlorobenzaldehyde, p-chlorobenzaldehyde, o-nitrobenzaldehyde, m-nitrobenzaldehyde, o-methylbenzaldehyde, m-methylbenzaldehyde, p-methylbenzaldehyde, p-ethylbenzaldehyde, p-n-butylbenzaldehyde, furfural, chloroacetaldehyde, and acetals derived from these, such as chloroacetaldehyde diethyl acetal. Preferred of these is formaldehyde.

These aldehydes may be used alone or in combination of two or more thereof. Examples of the acid catalyst include hydrochloric acid, sulfuric acid, formic acid, acetic acid, and oxalic acid.

The weight-average molecular weight of the thus-obtained novolak resin suitably is from 1′000 to 30′000. If the weight-average molecular weight thereof is lower than 1′000, the film reduction at unexposed parts during development is liable to be large. If the weight-average molecular weight there of exceeds 50′000, the developing rate may be too low. The especially preferred range of the molecular weight of the novolak resin is from 2′000 to 20′000.

The poly(hydroxystyrene)s and derivatives and copolymers thereof shown above as alkali-soluble polymers other than novolak resins each have a weight-average molecular weight of 2′000 or higher, preferably from 4′000 to 200′000, more preferably from 5′000 to 50′000. From the standpoint of obtaining a polymer film having improved heat resistance, the weight-average molecular weight thereof is desirably at least 5′000 or higher.

Weight-average molecular weight in the context of the present invention is meant to be the one determined by gel permeation chromatography and calibrated for with polystyrene standard.

In the present invention the alkali-soluble polymers may be used as a mixture of two or more thereof. In the case where a mixture of an alkali-soluble polymer and the polymer having groups which decompose by the action of an acid to enhance solubility in an alkaline developing solution is used, the addition amount of the alkali-soluble polymer is preferably up to 80% by weight, more preferably up to 60% by weight, most preferably up to 40% by weight, based on the total amount of the photosensitive composition (excluding the solvent). The amount exceeding 80% by weight is undesirable because the resist pattern suffers a considerable decrease in thickness, resulting in poor images and low resolution.

In the case where an alkali-soluble polymer is used together with a dissolution inhibitor, without the polymer having groups which decompose by the action of an acid, to enhance solubility in an alkaline developing solution, the amount of the alkali-soluble polymer is preferably from 40% to 90% by weight, more preferably from 50 to 85% by weight, most preferably 60 to 80% by weight. If the amount thereof is smaller than 40% by weight, undesirable results such as reduced sensitivity are caused. On the other hand, if it exceeds 90% by weight, the resist pattern suffers a considerable decrease in film thickness, resulting in poor resolution and image reproduction.

The use of the sulfonium salt derivatives according to the invention in chemically amplified systems, which operates on the principle of the removal of a protecting group from a polymer, generally produces a positive resist. Positive resists are preferred over negative resists in many applications, especially because of their higher resolution. There is, however, also interest in producing a negative image using the positive resist mechanism, in order to combine the advantages of the high degree of resolution of the positive resist with the properties of the negative resist. This can be achieved by introducing a so-called image-reversal step as described, for example, in EP 361906. For this purpose, the image-wise irradiated resist material is before the developing step treated with, for example, a gaseous base, thereby image-wise neutralizing the acid which has been produced. Then, a second irradiation, over the whole area, and thermal aftertreatment are carried out and the negative image is then developed in the customary manner.

The compounds of the formula I, and the polymers comprising repeating units derived from a compound of the formula I according to the present invention are in particular suitable as photolatent acids in the ArF resist technology, i.e. a technology using ArF excimer lasers (193 nm) for the imaging step. This technology requests the use of specific polymers/copolymers. Suitable formulations and the preparation of suitable polymer/copolymers are for example published in

Proceeding of SPIE 2438, 474 (1995); Proceeding of SPIE 3049, 44 (1997); Proceeding of SPIE 3333, 144 (1998); J. Photopolym. Sci. Technol. 14, 631 (2001); Proceeding of SPIE 3333, 546 (1998); J. Photopolym. Sci. Technol. 13, 601 (2000); JP2001-242627A; JP2001-290274A; JP2001-235863A; JP2001-228612A; Proceeding of SPIE 3333, 144 (1998); JP2001-5184A, commercially available as Lithomax alpha-7K from Mitsubishi Rayon; JP2001-272783A; U.S. patent application Ser. No. 09/413,763 (filed Oct. 7, 1999); EP 1091249; JP2000-292917A; JP2003-241385A; J. Photopolym. Sci. Technol. 14, 631 (2001); Proceeding of SPIE 3333, 11 (1998); ACS 1998 (University of Texas); JP2001-290274A; JP2001-235863A; JP2001-228612A; Proceeding of SPIE 3999, 13 (2000); JP2001-296663A; U.S. patent application Ser. No. 09/567,814 (filed 2000.5.9); EP 1128213; Proceeding of SPIE 3049, 104 (1997); J. Photopolym. Sci. Technol. 10, 521 (1997); JP2001-290274A; JP2001-235863A; JP2001-228612A; Proceeding of SPIE 4345, 680 (2001); J. Vac. Sci. Technol. B 16(6), p. 3716, 1998; Proceeding of SPIE 2724, 356 (1996); Proceeding of SPIE 4345, 67 (2001); Proceeding of SPIE 3333, 546 (1998); Proceeding of SPIE 4345, 87 (2001); Proceeding of SPIE 4345, 159 (2001); Proceeding of SPIE 3049, 92 (1997); Proceeding of SPIE 3049, 92 (1997); Proceeding of SPIE 3049, 92 (1997); Proceeding of SPIE 3999, 2 (2000); Proceeding of SPIE 3999, 23 (2000); Proceeding of SPIE 3999, 54 (2000); Proceeding of SPIE 4345, 119 (2001).

The formulations disclosed in the aforementioned publications are incorporated herein by reference. It is understood, that the compounds of the present invention are in particular suitable for use as photolatent acid in all the polymers/copolymers and compositions described in these cited publications.

The compounds of the formula I, and the polymers comprising repeating units derived from a compound of the formula I according to the present invention are suitable as photolatent acids in the bi-layer resist. This technology requests the use of specific polymers/copolymers. Suitable formulations and the preparation of suitable polymer/copolymers are for example published in Proc. SPIE 4345, 361-370 (2001), Proc. SPIE 4345, 406-416 (2001), JP-A-2002-278073, JP-A-2002-30116, JP-A-2002-30118, JP-A-2002-72477, JP-A-2002-348332, JP-A-2003-207896, JP-A-2002-82437, US2003/65101, US2003/64321.

The compounds of the formula I, and the polymers comprising repeating units derived from a compound of the formula I according to the present invention are suitable as photolatent acids in the multi-layer resist. This technology requests the use of specific polymers/copolymers. Suitable formulations and the preparation of suitable polymer/copolymers are for example published in JP-A-2003-177540, JP-A-2003-280207, JP-A-2003-149822, JP-A-2003-177544.

In order to make fine hole pattern, thermal flow process or chemical shrink technology, so-called RELACS (resolution enhancement lithography assisted by chemical shrink) process, are applied for chemically amplified resist. The compounds of the formula I, and the polymers comprising repeating units derived from a compound of the formula I according to the present invention are suitable as photolatent acids in the resists for thermal flow process or RELACS process. These technologies request the use of specific polymers/copolymers. Suitable formulations and the preparation of suitable polymer/copolymers are for example published in JP-A-2003-167357, JP-A-2001-337457, JP-A-2003-66626, US2001/53496, Proceeding of SPIE 5039, 789 (2003), IEDM98, Dig., 333 (1998), Proceeding Silicon Technology 11, 12 (1999),

The compounds of the formula I, and the polymers comprising repeating units derived from a compound of the formula I according to the present invention are suitable as photolatent acids in the F₂ resist technology, i.e. a technology using F₂ excimer lasers (157 nm) for the imaging step. This technology requests the use of specific polymers/copolymers which have high transparency at 157 nm. Examples of polymer suitable for this application are fluoropolymers described in, for example, Proc. SPIE 3999, 330-334 (2000), Proc. SPIE 3999, 357-364 (2000), Proc. SPIE 4345, 273-284 (2001), Proc. SPIE 4345, 285-295 (2001), Proc. SPIE 4345, 296-307 (2001), Proc. SPIE 4345, 327-334 (2001), Proc. SPIE 4345, 350-360 (2001), Proc. SPIE 4345, 379-384 (2001), Proc. SPIE 4345, 385-395 (2001), Proc. SPIE 4345, 417-427 (2001), Proc. SPIE 4345, 428-438 (2001), Proc. SPIE 4345, 439-447 (2001), Proc. SPIE 4345, 1048-1055 (2001), Proc. SPIE 4345, 1066-1072 (2001), Proc. SPIE 4690, 191-199 (2002), Proc. SPIE 4690, 200-211 (2002), Proc. SPIE 4690, 486-496 (2002), Proc. SPIE 4690, 497-503 (2002), Proc. SPIE 4690, 504-511 (2002), Proc. SPIE 4690, 522-532 (2002), US 20020031718, US 20020051938, US 20020055060, US 20020058199, US 20020102490, US 20020146639, US 20030003379, US 20030017404, WO 2002021212, WO 2002073316, WO 2003006413, JP-A-2001-296662, JP-A-2001-350263, JP-A-2001-350264, JP-A-2001-350265, JP-A-2001-356480, JP-A-2002-60475, JP-A-2002-90996, JP-A-2002-90997, JP-A-2002-155112, JP-A-2002-155118, JP-A-2002-155119, JP-A-2002-303982, JP-A-2002-327013, JP-A-2002-363222, JP-A-2003-2925, JP-A-2003-15301, JP-A-2003-2925, JP-A-2003-177539, JP-A-2003-192735, JP-A-2002-155115, JP-A-2003-241386, JP-A-2003-255544, US2003/36016, US2002/81499. Other suitable polymer for F₂ resist is silicon-containing polymers described in, for example, Proc. SPIE 3999, 365-374 (2000), Proc. SPIE 3999, 423-430 (2000), Proc. SPIE 4345, 319-326 (2001), US 20020025495, JP-A-2001-296664, JP-A-2002-179795, JP-A-2003-20335, JP-A-2002-278073, JP-A-2002-55456, JP-A-2002-348332. Polymers containing (meth)acrylonitrile monomer unit described in, for example, JP-A-2002-196495 is also suitable for F₂ resist.

The compounds of the formula I, and the polymers comprising repeating units derived from a compound of the formula I according to the present invention are suitable as photolatent acids in the EUV resist, i.e. a technology using light source of extreme ultra violet (13 nm) for the imaging step. This technology requests the use of specific polymers/copolymers. Suitable formulations and the preparation of suitable polymer/copolymers are for example published in JP-A-2002-55452, JP-A-2003-177537, JP-A-2003-280199, JP-A-2002-323758, US2002/51932.

The compounds of the formula I, and the polymers comprising repeating units derived from a compound of the formula I according to the present invention are suitable as photolatent acids in the EB (electron beam) or X-ray resist, i.e. a technology using EB or X-ray for the imaging step. These technologies request the use of specific polymers/copolymers. Suitable formulations and the preparation of suitable polymer/copolymers are for example published in JP-A-2002-99088, JP-A-2002-99089, JP-A-2002-99090, JP-A-2002-244297, JP-A-2003-5355, JP-A-2003-5356, JP-A-2003-162051, JP-A-2002-278068, JP-A-2002-333713, JP-A-2002-31892.

The compounds of the formula I, and the polymers comprising repeating units derived from a compound of the formula I according to the present invention are suitable as photolatent acids in the chemically amplified resist for immersion lithography. This technology reduces minimum feature size of resist pattern using liquid medium between the light source and the resist as described in Proceeding of SPIE 5040, 667 (2003), Proceeding of SPIE 5040, 679 (2003), Proceeding of SPIE 5040, 690 (2003), Proceeding of SPIE 5040, 724 (2003).

The compounds of the formula I, and the polymers comprising repeating units derived from a compound of the formula I according to the present invention are suitable as photolatent acids in the positive and negative photosensitive polyimide. This technology requests the use of specific polymers/copolymers. Suitable formulations and the preparation of suitable polymer/copolymers are for example published in JP-A-9-127697, JP-A-10-307393, JP-A-10-228110, JP-A-10-186664, JP-A-11-338154, JP-A-11-315141, JP-A-11-202489, JP-A-11-153866, JP-A-11-84653, JP-A-2000-241974, JP-A-2000-221681, JP-A-2000-34348, JP-A-2000-34347, JP-A-2000-34346, JP-A-2000-26603, JP-A-2001-290270, JP-A-2001-281440, JP-A-2001-264980, JP-A-2001-255657, JP-A-2001-214056, JP-A-2001-214055, JP-A-2001-166484, JP-A-2001-147533, JP-A-2001-125267, JP-A-2001-83704, JP-A-2001-66781, JP-A-2001-56559, JP-A-2001-33963, JP-A-2002-356555, JP-A-2002-356554, JP-A-2002-303977, JP-A-2002-284875, JP-A-2002-268221, JP-A-2002-162743, JP-A-2002-122993, JP-A-2002-99084, JP-A-2002-40658, JP-A-2002-37885, JP-A-2003-26919.

The formulations disclosed in the aforementioned publications are incorporated herein by reference. It is understood, that the compounds of the present invention are in particular suitable for use as photolatent acid in all the polymers/copolymers and compositions described in these cited publications.

Acid-sensitive components that produce a negative resist characteristically are especially compounds which, when catalysed by an acid (e.g. the acid formed during irradiation of the compounds of formulae I, or the polymers comprising repeating units derived from a compound of the formula I are capable of undergoing a crosslinking reaction with themselves and/or with one or more further components of the composition. Compounds of this type are, for example, the known acid-curable resins, such as, for example, acrylic, polyester, alkyd, melamine, urea, epoxy and phenolic resins or mixtures thereof. Amino resins, phenolic resins and epoxy resins are very suitable. Acid-curable resins of this type are generally known and are described, for example, in “Ullmann's Encyclopädie der technischen Chemie” [Ullmanns Enceclopedia of Technical Chemistry], 4th Edition, Vol. 15 (1978), p. 613-628. The crosslinker components should generally be present in a concentration of from 2 to 40, preferably from 5 to 30, percent by weight, based on the total solids content of the negative resist composition.

Subject of the invention also is a chemically amplified negative photoresist composition.

The invention also pertains to a chemically amplified negative photoresist composition, comprising

(a5) a component which, when catalysed by an acid undergoes a crosslinking reaction with itself and/or with the other components; and
(b) as photosensitive acid donor, at least one compound of the formula I and/or a polymer comprising at least one repeating unit derived from a compound of the formula I and optionally repeating units derived from ethylenically unsaturated compounds of formula II.

The invention includes, as a special embodiment, chemically amplified negative, alkali-developable photoresists, comprising

(a4) an alkali-soluble resin as binder
(a5) a component that when catalysed by an acid undergoes a crosslinking reaction with itself and/or with the binder, and
(b) as photosensitive acid donor at least one compound of the formula I and/or polymer comprising at least one repeating unit derived from a compound of the formula I and optionally repeating units derived from ethylenically unsaturated compounds selected from the group of formula II.

The composition may comprise additionally to the component (b) or components (a) and (b) [or components (a1), (a2), (a3) and (b), or components (a5) and (b), or components (a4), (a5) and (b)] other photosensitive acid donors (b1), other photoinitiators (d) and/or sensitizers (e) and optionally (c) other additives.

Especially preferred as acid-curable resins (a5) are amino resins, such as non-etherified or etherified melamine, urea, guanidine or biuret resins, especially methylated melamine resins or butylated melamine resins, corresponding glycolurils and urones. By “resins” in this context, there are to be understood both customary technical mixtures, which generally also comprise oligomers, and pure and high purity compounds. N-hexa(methoxymethyl) melamine and tetramethoxymethyl glucoril and N,N′-dimethoxymethylurone are the acid-curable resins given the greatest preference.

The concentration of the compound of formula I in negative resists in general is from 0.1 to 30, preferably up to 20, percent by weight, based on the total solids content of the compositions. From 1 to 15 percent by weight is especially preferred.

Where appropriate, the negative compositions may comprise a film-forming polymeric binder (a4). This binder is preferably an alkali-soluble phenolic resin. Well suited for this purpose are, for example, novolaks, derived from an aldehyde, for example acetaldehyde or furfuraldehyde, but especially from formaldehyde, and a phenol, for example unsubstituted phenol, mono- or di-chlorosubstituted phenol, such as p-chlorophenol, phenol mono- or di-substituted by C₁-C₉alkyl, such as o-, m- or p-cresol, the various xylenols, p-tert-butylphenol, p-nonylphenol, p-phenylphenol, resorcinol, bis(4-hydroxyphenyl)methane or 2,2-bis(4-hydroxyphenyl)propane. Also suitable are homo- and co-polymers based on ethylenically unsaturated phenols, for example homopolymers of vinyl- and 1-propenyl-substituted phenols, such as p-vinylphenol or p- (1-propenyl)phenol or copolymers of these phenols with one or more ethylenically unsaturated materials, for example styrenes. The amount of binder should generally be from 30 to 95 percent by weight or, preferably, from 40 to 80 percent by weight.

Sulfonium salt derivatives can also be used as acid generators, which can be activated photochemically, for the acid-catalysed crosslinking of, for example, poly(glycidyl)-methacrylates in negative resist systems. Such crosslinking reactions are described, for example, by Chae et al. in Pollimo 1993, 17(3), 292.

Suitable formulations and the preparation of suitable polymer/copolymers for the negative resist using the compounds of the formula I, and the polymers comprising repeating units derived from a compound of the formula I according to the present invention are for example published in JP-A-2003-43688, JP-A-2003-114531, JP-A-2002-287359, JP-A-2001-255656, JP-A-2001-305727, JP-A-2003-233185, JP-A-2003-186195, U.S. Pat. No. 6,576,394.

The subject composition includes, as a special embodiment, chemically amplified negative, solvent-developable photoresists, comprising

(a61) components that when catalysed by an acid undergo a crosslinking reaction or a polymerization with themselves and/or with other components, and
(b) as photosensitive acid donor a sulfonium salt of formula I.

The composition may comprise additionally to the component (b) other photosensitive acid donors (b1), other photoinitiators (d), other additives (c) and/or other binder resin (f).

The chemically amplified negative, solvent-developable photoresists request the use of a specific component that when catalysed by an acid undergoes a crosslinking reaction or a polymerization with itself and/or with other components in the formulation. Suitable formulations are for example published in U.S. Pat. No. 4,882,245, U.S. Pat. No. 5,026,624, U.S. Pat. No. 6,391,523.

A suitable component (a61) that when catalysed by an acid undergoes a crosslinking reaction or a polymerization with itself and/or with other components includes, for example, an epoxidized bisphenol A formaldehyde novolak resin and an epoxidized tetrabromo bisphenol A formaldehyde novolak resin. The preferred epoxy resin contains an average of eight epoxy groups, consisting of the glycidyl ether of the novolak condensation product of bisphenol A and formaldehyde, with an average molecular weight of about 1400 gram/mole, with an epoxy equivalent weight of about 215 gram/mole. Such a resin is, for example, commercially available from Shell Chemical under the trade name EPON® Resin SU-8.

Various kinds of polymers can be used as the binder resin (f) in the chemically amplified negative solvent-developable photoresist. Suitable examples include a phenoxy polyol resin which is a condensation product between epichlorohydrin and bisphenol A. A resin of this type is, for example, sold by Union Carbide Corporation under the Trade Mark PKHC.

The positive and the negative resist compositions may comprise in addition to the photosensitive acid donor compound of formula I, or the polymers comprising repeating units derived from a compound of the formula I further photosensitive acid donor compounds (b1), further additives (c), other photoinitiators (d), and/or sensitizers (e).

Therefore, subject of the invention also are chemically amplified resist compositions as described above, in addition to components (a) and (b), or components (a1), (a2), (a3) and (b), or components (a4), (a5) and (b) comprising further additives (c), further photosensitive acid donor compounds (b1), other photoinitiators (d), and/or sensitizers (e).

Sulfonium salt derivatives of the present invention in the positive and negative resist can also be used together with other, known photolatent acids (b1), for example, onium salts, 6-nitrobenzylsulfonates, bis-sulfonyl diazomethane compounds, cyano group-containing oxime sulfonate compounds, etc. Examples of known photolatent acids for chemically amplified resists are described in U.S. Pat. No. 5,731,364, U.S. Pat. No. 5,800,964, EP 704762, U.S. Pat. No. 5,468,589, U.S. Pat. No. 5,558,971, U.S. Pat. No. 5,558,976, U.S. Pat. No. 6,004,724, GB 2348644 and particularly in EP 794457 and EP 795786.

If a mixture of photolatent acids is used in the resist compositions according to the invention, the weight ratio of sulfonium salt derivatives of formula I, or the polymers comprising repeating units derived from a compound of the formula I to the other photolatent acid (b1) in the mixture is preferably from 1:99 to 99:1.

Examples of photolatent acids which are suitable to be used in admixture with the compounds of formula I, or the polymers comprising repeating units derived from a compound of the formula I are

(1) onium salt compounds, for example,
iodonium salts, sulfonium salts, phosphonium salts, diazonium salts, pyridinium salts. Preferred are diphenyliodonium triflate, diphenyliodonium pyrenesulfonate, diphenyliodonium dodecylbenzenesulfonate, triphenylsulfonium triflate, triphenylsulfonium hexafluoroantimonate, diphenyliodonium hexafluoroantimonate, triphenylsulfonium naphthalenesulfonate, (hydroxyphenyl)benzylmethylsulfonium toluenesulfonate, bis(4-tert-butylphenyl)iodonium bis(nonafluorobutanesulfonyl)imide, bis(4-tert-butylphenyl)iodonium tris(trifluoromethanesulfonyl)methide, triphenylsulfonium bis(trifluoromethanesulfonyl)imide, triphenylsulfonium (octafluorobutane-1,4-disulfonyl)imide, triphenylsulfonium tris(trifluoromethanesulfonyl)-methide, tert-butyldiphenylsulfonium tris(trifluoromethanesulfonyl)methide, triphenylsulfonium 1,3-disulfonylhexafluoropropyleneimide, triarylsulfonium tetrakis-(pentafluorophenyl) borates, e.g. triphenylsulfonium tetrakis-(pentafluorophenyl)borate, diaryliodonium tetrakis(pentafluorophenyl)borates, e.g. diphenyl tetrakis(pentafluorophenyl) borate, diphenyl-[4-(phenylthio)phenyl]sulfonium trifluorotris(pentafluoroethyl)phosphate and the like; the iodonium cation may also be 4-methylphenyl-4′-isobutylphenyliodonium or 4-methylphenyl-4′-isopropylphenyliodonium. Particularly preferred are triphenylsulfonium triflate, diphenyliodonium hexafluoroantimonate. Other examples are described in JP-A-2002-229192, JP-A-2003-140332, JP-A-2002-128755, JP-A-2003-35948, JP-A-2003-149800, JP-A-2002-6480, JP-A-2002-116546, JP-A-2002-156750, U.S. Pat. No. 6,458,506, US2003/27061, U.S. Pat. No. 5,554,664, WO2007-118794.
(2) halogen-containing compounds
haloalkyl group-containing heterocyclic compounds, haloalkyl group-containing hydrocarbon compounds and the like. Preferred are (trichloromethyl)-s-triazine derivatives such as phenyl-bis(trichloromethyl)-s-triazine, methoxyphenyl-bis(trichloromethyl)-s-triazine, naphthyl-bis-(trichloromethyl)-s-triazine and the like; 1,1-bis(4-chlorophenyl)-2,2,2-trichloroethane; and the like.
(3) sulfone compounds, for example of the formula

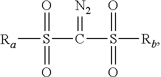

wherein Rₐ and R_{b} in-dependently of one another are alkyl, cycloalkyl or aryl, each of which may have at least one substituent, e.g.

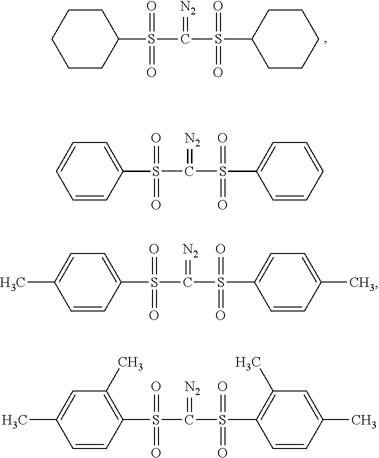

Such compounds are disclosed for example in US 2002/0172886-A, JP-A-2003-192665, US200219663. More examples are β-ketosulfones, β-sulfonylsulfones and their α-diazo derivatives and the like. Preferred are phenacylphenylsulfone, mesitylphenacylsulfone, bis(phenylsulfonyl)methane, bis(phenylsulfonyl)diazomethane.
(4) sulfonate compounds, for example
alkylsulfonic acid esters, haloalkylsulfonic acid esters, arylsulfonic acid esters, iminosulfonates, imidosulfonates and the like. Preferred imidosulfonate compounds are, for example, N-(trifluoromethylsulfonyloxy)succinimide, N-(trifluoromethylsulfonyloxy)phthalimide, N-(trifluoromethylsulfonyloxy)naphthylimide, N-(trifluoromethylsulfonyloxy)diphenylmaleimide, N-(trifluoromethylsulfonyloxy)-bicyclo-[2,2,1]-hept-5-ene-2,3-dicarboximide, N-(trifluoromethylsulfonyloxy)-7-oxabicyclo-[2,2,1]-hept-5-ene-2,3-dicarboximide, N-(trifluoromethylsulfonyloxy)-7-oxabicyclo-[2,2,1]-hept-5-ene-2,3-dicarboximide, N-(trifluoromethylsulfonyloxy)-bicyclo-[2,2,1]-heptan-5,6-oxy-2,3-dicarboximide, N-(camphanylsulfonyloxy) succinimide, N-(camphanylsulfonyloxy)phthalimide, N-(camphanylsulfonyloxy)naphthylimide, N-(camphanylsulfonyloxy)diphenylmaleimide, N-(camphanylsulfonyloxy)-bicyclo-[2,2,1]-hept-5-ene-2,3-dicarboximide, N-(camphanylsulfonyloxy)-7-oxabicyclo-[2,2,1]-hept-5-ene-2,3-dicarboximide, N-(camphanylsulfonyloxy)-7-oxabicyclo-[2,2,1]hept-5-ene-2,3-dicarboximide, N-(camphanylsulfonyloxy)-bicyclo-[2,2,1]-heptan-5,6-oxy-2,3-dicarboximide, N-(4-methylphenylsulfonyloxy)succinimide, N-(4-methylphenylsulfonyloxy)phthalimide, N-(4-methylphenylsulfonyloxy)naphthylimide, N-(4-methylphenylsulfonyloxy)naphthylimide, N-(4-methylphenylsulfonyloxy)diphenylmaleimide, N-(4-methylphenylsulfonyloxy)-bicyclo-[2,2,1]-hept-5-ene-2,3-dicarboximide, N-(4-methylphenylsulfonyloxy)-7-oxabicyclo-[2,2,1]-hept-5-ene-2,3-dicarboximide, N-(4-methylphenylsulfonyloxy)-bicyclo-[2,2,1]-heptan-5,6-oxy-2,3-dicarboximide, N-(2-trifluoromethylphenylsulfonyloxy)succinimide, N-(2-trifluoromethylphenylsulfonyloxy)-naphthylimide, N-(2-trifluoromethylphenylsulfonyloxy)diphenylmaleimide, N-(2-trifluoromethylphenylsulfonyloxy)-bicyclo-[2,2,1]-hept-5-ene-2,3-dicarboximide, N-(2-trifluoromethylphenylsulfonyloxy)-7-oxabicyclo-[2,2,1]-hept-5-ene-2,3-dicarboximide, N-(2-trifluoromethylphenylsulfonyloxy)-bicyclo-[2,2,1]-heptan-5,6-oxy-2,3-dicarboximide and the like.

Other suitable sulfonate compounds preferably are, for example, benzoin tosylate, pyrogallol tristriflate, pyrogallolomethanesulfonic acid triester, nitorobenzyl-9,10-diethoxyanthracene-2-sulfonate, α-(4-toluene-sulfonyloxyimino)-benzyl cyanide, α-(4-toluene-sulfonyloxyimino)-4-methoxybenzyl cyanide, α-(4-toluene-sulfonyloxyimino)-2-thienylmethyl cyanide, α-(methanesulfonyloxyimino)-1-cyclohexenylacetonitrile, α-(butylsulfonyloxyimino)-1-cyclo-pentenylacetonitrile, (4-methylsulfonyloxyimino-cyclohexa-2,5-dienylidene)-phenylacetonitrile, (5-methylsulfonyloxyimino-5H-thiophen-2-ylidene)-phenyl-acetonitrile, (5-methylsulfonyloxyimino-5H-thiophen-2-ylidene)-(2-methylphenyl)-acetonitrile, (5-propylsulfonyloxyimino-5H-thiophen-2-ylidene)-(2-methylphenyl)-acetonitrile, (5-(p-toluenesulfonyloxyimino)-5H-thiophen-2-ylidene)-(2-methylphenyl)-acetonitrile, (5-(10-camphorsulfonyloxyimino)-5H-thiophen-2-ylidene)-(2-methylphenyl)-acetonitrile, (5-methylsulfonyloxyimino-5H-thiophen-2-ylidene)-(2-chlorophenyl)-acetonitrile, 2,2,2-trifluoro-1-{4-(3-[4-{2,2,2-trifluoro-1-(1-propanesulfonyl-oxyimino)-ethyl}-phenoxy]-propoxy)-phenyl}-ethanone oxime 1-propanesulfonate, 2,2,2-trifluoro-1-{4-(3-[4-{2,2,2-trifluoro-1-(1-p-toluenesulfonyloxyimino)-ethyl}-phenoxy]propoxy)-phenyl}-ethanone oxime 1-p-toluenesulfonate, 2-[2,2,3,3,4,4,5,5,6,6,7,7-dodecafluoro-1-(nonafluorobutylsulfonyloxyimino)-heptyl]-fluorene, 2-[2,2,3,3,4,4,4-heptafluoro-1-(nonafluorobutylsulfonyloxyimino)-butyl]-fluorene, 2-[2,2,3,3,4,4,5,5-octafluoro-1-(nonafluorobutylsulfonyloxyimino)-pentyl]-fluorene, 8-[2,2,3,3,4,4,5,5-octafluoro-1-(nonafluorobutylsulfonyloxyimino)-pentyl]-fluoranthene and the like.

In the radiation sensitive resin composition of this invention, particularly preferred sulfonate compounds include pyrogallolmethanesulfonic acid triester, N-(trifluoromethylsulfonyloxy)bicyclo-[2,2,1]-hept-5-ene-2,3-dicarboximide, N-(camphanylsulfonyloxy)naphthylimide, N-(2-trifluoromethylphenylsulfonyloxy)phthalimide, N-(trifluoromethylsulfonyloxy)-bicyclo-[2,2,1]-hept-5-ene-2,3-dicarboximide, N-(camphanylsulfonyloxy)naphthylimide, N-(2-trifluoromethylphenylsulfonyloxy)phthalimide and the like.

(5) Quinonediazide compounds, for example
1,2-quinonediazidesulfonic acid ester compounds of polyhydroxy compounds. Preferred are compounds having a 1,2-quinonediazidesulfonyl group, e.g. a 1,2-benzoquinonediazide-4-sulfonyl group, a 1,2-naphthoquinonediazide-4-sulfonyl group, a 1,2-naphthoquinonediazide-5-sulfonyl group, a 1,2-naphthoquinonediazide-6-sulfonyl group or the like. Particularly preferred are compounds having a 1,2-naphthoquinonediazide-4-sulfonyl group or a 1,2-naphthoquinonediazide-5-sulfonyl group. In particular suitable are 1,2-quinonediazidesulfonic acid esters of (poly)hydroxyphenyl aryl ketones such as 2,3,4-trihydroxybenzophenone, 2,4,6-trihydroxybenzophenone, 2,3,4,4′-tetrahydroxybenzophenone, 2,2′,3,4-tetrahydroxybenzophenone, 2,3,4,4′-tetrahydroxybenzophenone, 2,2′,4,4′-tetrahydroxybenzophenone 2,2′,3,4,4′-pentahydroxybenzophenone, 2,2′3,2,6′-pentahydroxybenzophenone, 2,3,3′,4,4′5′-hexahydroxybenzophenone, 2,3′,4,4′,5′6-hexahydroxybenzophenone and the like; 1,2-quinonediazidesulfonic acid esters of bis-[(poly)hydroxylphenyl]alkanes such as bis(4-hydroxyphenyl)ethane, bis(2,4-dihydroxyphenyl)ethane, 2,2-bis(4-hydroxyphenyl)propane, 2,2-bis(2,4-dihydroxyphenyl)propane, 2,2-bis-(2,3,4-trihydroxyphenyl)propane and the like; 1,2-quinonediazidesulfonic acid esters of (poly)hydroxyphenylalkanes such as 4,4′-dihydroxytriphenylmethane, 4,4′4″-trihydroxytriphenylmethane, 4,4′5,5′-tetramethyl-2,2′2″-trihydroxytriphenylmethane, 2,2,5,5′-tetramethyl-4,4′,4″-trihydroxytriphenylmethane, 1,1,1-tris(4-hydroxyphenyl)ethane, 1,1-bis(4-hydroxyphenyl)-1-phenylethane, 1,1-bis(4-hydroxyphenyl)-1-(4-[1-(hydroxyphenyl)-1-methylethyl]phenyl)ethane and the like; 1,2-quinonediazidesulfonic acid esters of (poly)hydroxylphenylflavans such as 2,4,4-trimethyl-2′,4′,7-trihydroxy-2-phenylflavan, 2,4,4-trimethyl-2′,4′,5′,6,7-pentahydroxy-2-phenylflavan and the like.

Other examples of photolatent acids which are suitable to be used in admixture with the compounds according to the present invention are described in JP-A-2003-43678, JP-A-2003-5372, JP-A-2003-43677, JP-A-2002-357904, JP-A-2002-229192.

The positive and negative photoresist composition of the present invention may optionally contain one or more additives (c) customarily used in photoresists in the customary amounts known to a person skilled in the art, for example, dyes, pigments, plasticizers, surfactants, flow improvers, wetting agents, adhesion promoters, thixotropic agents, colourants, fillers, solubility accelerators, acid-amplifier, photosensitizers and organic basic compounds.

Further examples for organic basic compounds which can be used in the resist composition of the present invention are compounds which are stronger bases than phenol, in particular, nitrogen containing basic compounds. These compounds may be ionic, like, for example, tetraalkylammonium salts or non-ionic. Preferred organic basic compounds are nitrogen-containing basic compounds having, per molecule, two or more nitrogen atoms having different chemical environments. Especially preferred are compounds containing both at least one substituted or unsubstituted amino group and at least one nitrogen-containing ring structure, and compounds having at least one alkylamino group. Examples of such preferred compounds include guanidine, aminopyridine, amino alkylpyridines, aminopyrrolidine, indazole, imidazole, pyrazole, pyrazine, pyrimidine, purine, imidazoline, pyrazoline, piperazine, aminomorpholine, and aminoalkylmorpholines. Suitable are both, the unsubstituted compounds or substituted derivatives thereof. Preferred substituents include amino, aminoalkyl groups, alkylamino groups, aminoaryl groups, arylamino groups, alkyl groups alkoxy groups, acyl groups acyloxy groups aryl groups, aryloxy groups, nitro, hydroxy, and cyano. Specific examples of especially preferred organic basic compounds include guanidine, 1,1-dimethylguanidine, 1,1,3,3-tetramethylguanidine, 2-aminopyridine, 3-aminopyridine, 4-aminopyridine, 2-dimethylaminopyridine, 4-dimethylaminopyridine, 2-diethylaminopyridine, 2-(aminomethyl)-pyridine, 2-amino-3-methylpyridine, 2-amino-4-methylpyridine, 2-amino-5-methylpyridine, 2-amino-6-methylpyridine, 3-aminomethylpyridine, 4-aminomethylpyridine, 3-aminopyrrolidine, piperazine, N-(2-aminoethyl)piperazine, N-(2-aminoethyl)piperidine, 4-amino-2,2,6,6-tetramethylpiperidine, 4-piperidinopiperidine, 2-imimopiperidine, 1-(2-aminoethyl)pyrrolidine, pyrazole, 3-amino-5-methylpyrazole, 5-amino-3-methyl-1-p-tolylpyrazole, pyrazine, 2-(aminomethyl)-5-methylpyrazine, pyrimidine, 2,4-diaminopyrimidine, 4,6-dihydroxypyrimidine, 2-pyrazoline, 3-pyrazoline, N-aminomorpholine, and N-(2-aminoethyl)morpholine.

Other examples of suitable organic basic compounds are described in DE 4408318, U.S. Pat. No. 5,609,989, U.S. Pat. No. 5,556,734, EP 762207, DE 4306069, EP 611998, EP 813113, EP 611998, and U.S. Pat. No. 5,498,506, JP-A-2003-43677, JP-A-2003-43678, JP-A-2002-226470, JP-A-2002-363146, JP-A-2002-363148, JP-A-2002-363152, JP-A-2003-98672, JP-A-2003-122013, JP-A-2002-341522. However, the organic basic compounds suitable in the present invention are not limited to these examples.

The nitrogen-containing basic compounds may be used alone or in combination of two or more thereof. The added amount of the nitrogen-containing basic compounds is usually from 0.001 to 10 parts by weight, preferably from 0.01 to 5 parts by weight, per 100 parts by weight of the photosensitive resin composition (excluding the solvent). If the amount thereof is smaller than 0.001 part by weight, the effects of the present invention cannot be obtained. On the other hand, if it exceeds 10 parts by weight, reduced sensitivity and impaired developability at unexposed parts are liable to be caused.

The composition can further contain a basic organic compound which decomposes under actinic radiation (“suicide base”) such as for example described in EP 710885, U.S. Pat. No. 5,663,035, U.S. Pat. No. 5,595,855, U.S. Pat. No. 5,525,453, and EP 611998.

Examples of dyes (c) suitable for the compositions of the present invention are oil-soluble dyes and basic dyes, e.g. Oil Yellow #101, Oil Yellow #103, Oil Pink #312, Oil Green BG, Oil Blue BOS, Oil Blue #603, Oil Black BY, Oil Black BS, Oil Black T-505 (all manufactured by Orient Chemical Industries Ltd., Japan), crystal violet (Cl 42555), methyl violet (Cl 42535), rhodamine B (Cl 45170B), malachite green (Cl 42000), and methylene blue (Cl 52015).

Spectral sensitizers (e) may be further added to sensitize the photo latent acid to exhibit absorption in a region of longer wavelengths than far ultraviolet, whereby the photosensitive composition of the present invention can, for example, be rendered sensitive to an i-line or g-line radiation. Examples of suitable spectral sensitizers include benzophenones, p,p′-tetramethyldiaminobenzophenone, p,p′-tetraethylethylaminobenzophenone, thioxanthone, 2-chlorothioxanthone, anthrone, pyrene, perylene, phenothiazine, benzil, acridine orange, benzoflavin, cetoflavin T, 9,10-diphenylanthracene, 9-fluorenone, acetophenone, phenanthrene, 2-nitrofluorene, 5-nitroacenaphthene, benzoquinone, 2-chloro-4-nitroaniline, N-acetyl-p-nitroaniline, p-nitroaniline, N-acetyl-4-nitro-1-naphthylamine, picramide, anthraquinone, 2-ethylanthraquinone, 2-tert-butylanthraquinone, 1,2-benzanthraquinone, 3-methyl-1,3-diaza-1,9-benzanthrone, dibenzalacetone, 1,2-naphthoquinone, 3-acylcoumarin derivatives, 3,3′-carbonyl-bis-(5,7-dimethoxycarbonylcoumarin), 3-(aroylmethylene) thiazolines, eosin, rhodamine, erythrosine, and coronene. However, the suitable spectral sensitizers are not limited to these examples.

These spectral sensitizers can be used also as light absorbers for absorbing the far ultraviolet emitted by a light source. In this case, the light absorber reduces light reflection from the substrate and lessens the influence of multiple reflection within the resist film, thereby diminishing the effect of standing waves.

Specific examples of such compounds are

### 1. Thioxanthones

Thioxanthone, 2-isopropylthioxanthone, 2-chlorothioxanthone, 1-chloro-4-propoxythioxanthone, 2-dodecylthioxanthone, 2,4-diethylthioxanthone, 2,4-dimethylthioxanthone, 1-methoxycarbonylthioxanthone, 2-ethoxycarbonylthioxanthone, 3-(2-methoxyethoxycarbonyl)-thioxanthone, 4-butoxycarbonylthioxanthone, 3-butoxycarbonyl-7-methylthioxanthone, 1-cyano-3-chlorothioxanthone, 1-ethoxycarbonyl-3-chlorothioxanthone, 1-ethoxycarbonyl-3-ethoxythioxanthone, 1-ethoxycarbonyl-3-aminothioxanthone, 1-ethoxycarbonyl-3-phenylsulfurylthioxanthone, 3,4-di-[2-(2-methoxyethoxy)ethoxycarbonyl]-thioxanthone, 1,3-dimethyl-2-hydroxy-9H-thioxanthen-9-one 2-ethylhexylether, 1-ethoxycarbonyl-3-(1-methyl-1-morpholinoethyl)-thioxanthone, 2-methyl-6-dimethoxymethyl-thioxanthone, 2-methyl-6-(1,1-dimethoxybenzyl)-thioxanthone, 2-morpholinomethylthioxanthone, 2-methyl-6-morpholinomethylthioxanthone, N-allylthioxanthone-3,4-dicarboximide, N-octylthioxanthone-3,4-dicarboximide, N-(1,1,3,3-tetramethylbutyl)-thioxanthone-3,4-dicarboximide, 1-phenoxythioxanthone, 6-ethoxycarbonyl-2-methoxythioxanthone, 6-ethoxycarbonyl-2-methylthioxanthone, thioxanthone-2-carboxylic acid polyethyleneglycol ester, 2-hydroxy-3-(3,4-dimethyl-9-oxo-9H-thioxanthone-2-yl-oxy)-N,N,N-trimethyl-1-propanaminium chloride;

### 2. Benzophenones

benzophenone, 4-phenyl benzophenone, 4-methoxy benzophenone, 4,4′-dimethoxy benzophenone, 4,4′-dimethyl benzophenone, 4,4′-dichlorobenzophenone 4,4′-bis(dimethylamino)-benzophenone, 4,4′-bis(diethylamino)benzophenone, 4,4′-bis(methylethylamino)benzophenone, 4,4′-bis(p-isopropylphenoxy)benzophenone, 4-methyl benzophenone, 2,4,6-trimethylbenzophenone, 3-methyl-4′-phenyl-benzophenone, 2,4,6-trimethyl-4′-phenyl-benzophenone, 4-(4-methylthiophenyl)-benzophenone, 3,3′-dimethyl-4-methoxy benzophenone, methyl-2-benzoylbenzoate, 4-(2-hydroxyethylthio)-benzophenone, 4-(4-tolylthio)benzophenone, 1-[4-(4-benzoyl-phenylsulfanyl)-phenyl]-2-methyl-2-(toluene-4-sulfonyl)-propan-1-one, 4-benzoyl-N,N,N-trimethylbenzenemethanaminium chloride, 2-hydroxy-3-(4-benzoylphenoxy)-N,N,N-trimethyl-1-propanaminium chloride monohydrate, 4-(13-acryloyl-1,4,7,10,13-pentaoxamidecyl)-benzophenone, 4-benzoyl-N,N-dimethyl-N-[2-(1-oxo-2-propenyl)oxy]-ethyl-benzenemethanaminium chloride;

### 3. Coumarins

Coumarin 1, Coumarin 2, Coumarin 6, Coumarin 7, Coumarin 30, Coumarin 102, Coumarin 106, Coumarin 138, Coumarin 152, Coumarin 153, Coumarin 307, Coumarin 314, Coumarin 314T, Coumarin 334, Coumarin 337, Coumarin 500, 3-benzoyl coumarin, 3-benzoyl-7-methoxycoumarin, 3-benzoyl-5,7-dimethoxycoumarin, 3-benzoyl-5,7-dipropoxycoumarin, 3-benzoyl-6,8-dichlorocoumarin, 3-benzoyl-6-chloro-coumarin, 3,3′-carbonyl-bis-[5,7-di(propoxy)-coumarin], 3,3′-carbonyl-bis(7-methoxycoumarin), 3,3′-carbonyl-bis(7-diethylamino-coumarin), 3-isobutyroylcoumarin, 3-benzoyl-5,7-dimethoxy-coumarin, 3-benzoyl-5,7-diethoxy-coumarin, 3-benzoyl-5,7-dibutoxycoumarin, 3-benzoyl-5,7-di(methoxyethoxy)-coumarin, 3-benzoyl-5,7-di(allyloxy)coumarin, 3-benzoyl-7-dimethylaminocoumarin, 3-benzoyl-7-diethylaminocoumarin, 3-isobutyroyl-7-dimethylaminocoumarin, 5,7-dimethoxy-3-(1-naphthoyl)-coumarin, 5,7-diethoxy-3-(1-naphthoyl)-coumarin, 3-benzoylbenzo[f]coumarin, 7-diethylamino-3-thienoylcoumarin, 3-(4-cyanobenzoyl)-5,7-dimethoxycoumarin, 3-(4-cyanobenzoyl)-5,7-dipropoxycoumarin, 7-dimethylamino-3-phenylcoumarin, 7-diethylamino-3-phenylcoumarin, the coumarin derivatives disclosed in JP 09-179299-A and JP 09-325209-A, for example 7-[{4-chloro-6-(diethylamino)-S-triazine-2-yl}amino]-3-phenylcoumarin;

4. 3-(aroylmethylene)-thiazolines

3-methyl-2-benzoylmethylene-β-naphthothiazoline, 3-methyl-2-benzoylmethylene-benzothiazoline, 3-ethyl-2-propionylmethylene-β-naphthothiazoline;

### 5. Rhodanines

4-dimethylaminobenzalrhodanine, 4-diethylaminobenzalrhodanine, 3-ethyl-5-(3-octyl-2-benzothiazolinylidene)-rhodanine, the rhodanine derivatives, formulae [1], [2], [7], disclosed in JP 08-305019A;

6. Other compounds
acetophenone, 3-methoxyacetophenone, 4-phenylacetophenone, benzil, 4,4′-bis(dimethylamino)benzil, 2-acetylnaphthalene, 2-naphthaldehyde, dansyl acid derivatives, 9,10-anthraquinone, anthracene, pyrene, aminopyrene, perylene, phenanthrene, phenanthrenequinone, 9-fluorenone, dibenzosuberone, curcumin, xanthone, thiomichler's ketone, α-(4-diethylaminobenzylidene) ketones, e.g. 2,5-bis(4-diethylaminobenzylidene)cyclopentanone, 2-(4-dimethylamino-benzylidene)-indan-1-one, 3-(4-dimethylamino-phenyl)-1-indan-5-yl-propenone, 3-phenylthiophthalimide, N-methyl-3,5-di(ethylthio)-phthalimide, N-methyl-3,5-di(ethylthio)-phthalimide, phenothiazine, methylphenothiazine, amines, e.g. N-phenylglycine, ethyl 4-dimethylaminobenzoate, butoxyethyl 4-dimethylaminobenzoate, 4-dimethylaminoacetophenone, triethanolamine, methyldiethanolamine, dimethylaminoethanol, 2-(dimethylamino)ethyl benzoate, poly(propyleneglycol)-4-(dimethylamino) benzoate, pyrromethenes, e.g., 1,3,5,7,9-pentamethylpyrromethene BF₂ complex, 2,8-diethyl-1,3,5,7,9-pentamethylpyrromethene BF₂ complex, 2,8-diethyl-5-phenyl-1,3,7,9-tetramethylpyrromethene BF₂ complex, 9,10-bis(phenylethynyl)-1,8-dimethoxyanthracene, benzo[1,2,3-kl:4,5,6-k′l′]dixanthene.

Further suitable additives (c) are “acid-amplifiers”, compounds that accelerate the acid formation or enhance the acid concentration. Such compounds may also be used in combination with the sulfonium salt derivatives of the formula I, or the polymers comprising repeating units derived from a compound of the formula I according to the invention in positive or negative resists, or in imaging systems as well as in all coating applications. Such acid amplifiers are described e.g. in Arimitsu, K. et al. J. Photopolym. Sci. Technol. 1995, 8, pp 43; Kudo, K. et al. J. Photopolym. Sci. Technol. 1995, 8, pp 45; Ichimura, K. et al. Chem: Letters 1995, pp 551.

Other additives (c) to improve the resist performance such as resolution, pattern profile, process latitude, line edge roughness, stability are described in JP-A-2002-122992, JP-A-2002-303986, JP-A-2002-278071, JP-A-2003-57827, JP-A-2003-140348, JP-A-2002-6495, JP-A-2002-23374, JP-A-2002-90987, JP-A-2002-91004, JP-A-2002-131913, JP-A-2002-131916, JP-A-2002-214768, JP-A-2001-318464, JP-A-2001-330947, JP-A-2003-57815, JP-A-2003-280200, JP-A-2002-287362, JP-A-2001-343750. Such compounds may also be used in combination with the sulfonium salt derivatives of the formula I, or the polymers comprising repeating units derived from a compound of the formula I according to the invention in positive or negative resists.

Usually, for the application to a substrate of the photosensitive composition of the present invention, the composition is dissolved in an appropriate solvent. Preferred examples of these solvents include ethylene dichloride, cyclohexanone, cyclopentanone, 2-heptanone, γ-butyrolactone, methyl ethyl ketone, ethylene glycol monomethyl ether, ethylene glycol monoethyl ether, 2-methoxyethyl acetate, 2-ethoxyethyl acetate, 2-ethoxyethanol, diethyl glycol dimethyl ether, ethylene glycol monoethyl ether acetate, propylene glycol monomethyl ether, propylene glycol monomethyl ether acetate, toluene, ethyl acetate, butyl acetate, methyl lactate, ethyl lactate, methyl methoxypropionate, ethyl ethoxypropionate, methyl pyruvate, ethyl pyruvate, propyl pyruvate, N,N-dimethylformamide, dimethyl sulfoxide, N-methylpyrrolidone, and tetrahydrofuran. These solvents may be used alone or as mixtures. Preferred examples of the solvents are esters, such as 2-methoxyethyl acetate, ethylene glycolmonoethyl ether acetate, propylene glycol monomethyl ether acetate, methyl methoxypropionate, ethyl ethoxypropionate, and ethyl lactate. Use of such solvents is advantageous because the sulfonium salt derivatives represented by formula I, or the polymers comprising repeating units derived from a compound of the formula I according to the present invention have good compatibility therewith and better solubility therein.

A surfactant can be added to the solvent. Examples of suitable surfactants include nonionic surfactants, such as polyoxyethylene alkyl ethers, e.g. polyoxyethylene lauryl ether, polyoxyethylene stearyl ether, polyoxyethylene acetyl ether, and polyoxyethylene oleyl ether; polyoxyethylene alkylaryl ethers, e.g. polyoxyethylene, octylphenol ether and polyoxyethylene non-ylphenol ether; polyoxyethylene/polyoxypropylene block copolymers, sorbitan/fatty acid esters, e.g. sorbitan monolaurate, sorbitan monopalmitate, sorbitan monostearate, sorbitan monooleate, sorbitan trioleate; fluorochemical surfactants such as F-top EF301, EF303, and EF352 (manufactured by New Akita Chemical Company, Japan). Megafac F171 and F17.3 (manufactured by Dainippon Ink & Chemicals, Inc, Japan), Fluorad FC 430 and FC431 (manufactured by Sumitomo 3M Ltd., Japan), Asahi Guard AG710 and Surflon S-382, SC101, SC102, SC103, SC104, SC105, and SC106 (manufactured by Asahi Grass Col, Ltd., Japan); organosiloxane polymer KP341 (manufactured by Shin-Etsu Chemical Co., Ltd., Japan); and acrylic or methacrylic (co)polymers Poly-flow Now.75 and NO.95 (manufactured by Kyoeisha Chemical Co., Ltd., Japan). Other examples are described in JP-A-2001-318459, JP-A-2002-6483. The added amount of the surfactant usually is 2 parts by weight or lower, desirably 0.5 part by weight or lower, per 100 parts by weight of the solid components of the composition of the present invention. The surfactants may be added alone or in combination of two or more thereof.

The solution is uniformly applied to a substrate by means of known coating methods, for example by spin-coating, immersion, knife coating, curtain pouring techniques, brush application, spraying and roller coating. It is also possible to apply the photosensitive layer to a temporary, flexible support and then to coat the final substrate by coating transfer (laminating). The amount applied (coating thickness) and the nature of the substrate (coating substrate) are dependent on the desired field of application. The range of coating thicknesses can in principle include values from approximately 0.01 μm to more than 100 μm.

After the coating operation generally the solvent is removed by heating, resulting in a layer of the photoresist on the substrate. The drying temperature must of course be lower than the temperature at which certain components of the resist might react or decompose. In general, drying temperatures are in the range from 60 to 160° C.

The resist coating is then irradiated image-wise. The expression “image-wise irradiation” includes irradiation in a predetermined pattern using actinic radiation, i.e. both irradiation through a mask containing a predetermined pattern, for example a transparency, a chrome mask or a reticle, and irradiation using a laser beam or electron beam that writes directly onto the resist surface, for example under the control of a computer, and thus produces an image. Another way to produce a pattern is by interference of two beams or images as used for example in holographic applications. It is also possible to use masks made of liquid crystals that can be addressed pixel by pixel to generate digital images, as is, for example described by A. Bertsch; J. Y. Jezequel; J. C. Andre in Journal of Photochemistry and Photobiology A: Chemistry 1997, 107 pp. 275-281 and by K. P. Nicolay in Offset Printing 1997, 6, pp. 34-37.

After the irradiation and, if necessary, thermal treatment, the irradiated sites (in the case of positive resists) or the non-irradiated sites (in the case of negative resists) of the composition are removed in a manner known per se using a developer.

In order to accelerate the catalytic reaction and hence the development of a sufficient difference in solubility between the irradiated and unirradiated sections of the resist coating in the developer, the coating is preferably heated before being developed. The heating can also be carried out or begun during the irradiation. Temperatures of from 60 to 160° C. are preferably used. The period of time depends on the heating method and, if necessary, the optimum period can be determined easily by a person skilled in the art by means of a few routine experiments. It is generally from a few seconds to several minutes. For example, a period of from 10 to 300 seconds is very suitable when a hotplate is used and from 1 to 30 minutes when a convection oven is used. It is important for the latent acid donors according to the invention in the unirradiated sites on the resist to be stable under those processing conditions.

The coating is then developed, the portions of the coating that, after irradiation, are more soluble in the developer being removed. If necessary, slight agitation of the workpiece, gentle brushing of the coating in the developer bath or spray developing can accelerate that process step. The aqueous-alkaline developers customary in resist technology may, for example, be used for the development. Such developers comprise, for example, sodium or potassium hydroxide, the corresponding carbonates, hydrogen carbonates, silicates or metasilicates, but preferably metal-free bases, such as ammonia or amines, for example ethylamine, n-propylamine, diethylamine, di-n-propylamine, triethylamine, methyl diethylamine, alkanolamines, for example dimethyl ethanolamine, triethanolamine, quaternary ammonium hydroxides, for example tetramethylammonium hydroxide or tetraethylammonium hydroxide. The developer solutions are generally up to 0.5 N, but are usually diluted in suitable manner before use. For example solutions having a normality of approximately 0.1-0.3 are well suited. The choice of developer depends on the nature of the photocurable surface coating, especially on the nature of the binder used or of the resulting photolysis products. The aqueous developer solutions may, if necessary, also comprise relatively small amounts of wetting agents and/or organic solvents. Typical organic solvents that can be added to the developer fluids are, for example, cyclohexanone, 2-ethoxyethanol, toluene, acetone, isopropanol and also mixtures of two or more of these solvents. A typical aqueous/organic developer system is based on Butylcellosolve®/water.

Subject of the invention also is a process for the preparation of a photoresist by

(1) applying to a substrate a composition as described above;
(2) post apply baking the composition at temperatures between 60° C. and 160° C.;
(3) image-wise irradiating with light of wavelengths between 10 nm and 1500 nm;
(4) optionally post exposure baking the composition at temperatures between 60° C. and 160° C.; and
(5) developing with a solvent or with an aqueous alkaline developer.

Preferred is a process, wherein the image-wise irradiation is carried out with monochromatic or polychromatic radiation in the wavelength range from 10 to 450 nm, in particular in the range from 10 to 260 nm.

The photoresist compositions can be used on all substrates and with all exposure techniques known to the person skilled in the art. For example, semiconductor substrates can be used, such as silicon, gallium arsenide, germanium, indium antimonide; furthermore substrate covered by oxide or nitride layers, such as silicon dioxide, silicon nitride, titanium nitride, siloxanes, as well as metal substrates and metal coated substrates with metals such as aluminium, copper, tungsten, etc. The substrate can also be coated with polymeric materials, for example with organic antireflective coatings, insulation layers and dielectric coatings from polymeric materials prior to coating with the photoresist.

The photoresist layer can be exposed by all common techniques, such as direct writing, i.e. with a laser beam or projection lithography in stepp- and repeat mode or scanning mode, or by contact printing through a mask.

In case of projection lithography a wide range of optical conditions can be used such as coherent, partial coherent or incoherent irradiation. This includes off-axis illumination techniques, for example annular illumination and quadruple illumination where the radiation is allowed to pass only certain regions of the lens, excluding the lens center.

The mask used to replicate the pattern can be a hard mask or a flexible mask. The mask can include transparent, semitransparent and opaque patterns. The pattern size can include also patterns which are at or below the resolution limit of the projection optics and placed on the mask in a certain way in order to modify the aerial image, intensity and phase modulation of the irradiation after having passed the mask. This includes phase shift masks and half-tone phase shift masks.

The patterning process of the photoresist composition can be used to generate patterns of any desired geometry and shape, for example dense and isolated lines, contact holes, trenches, dots, etc.

The photoresists according to the invention have excellent lithographic properties, in particular a high sensitivity, and high resist transparency for the imaging radiation.

Possible areas of use of the composition according to the invention are as follows: use as photoresists for electronics, such as etching resists, ion-implantation resist, electroplating resists or solder resists, the manufacture of integrated circuits or thin film transistor-resist (TFT); the manufacture of printing plates, such as offset printing plates or screen printing stencils, use in the etching of moldings or in stereolithography or holography techniques, which are employed for various applications, for example, 3D optical information storage described in J. Photochem. Photobio. A, 158, 163 (2003), Chem. Mater. 14, 3656 (2002). The composition according to the invention is also suitable for making inter-metal dielectrics layer, buffer layer, passivation coat of semiconductor devices and suitable for making waveguide for optoelectronics. For MEMS (micro electro mechanical systems) application, the composition according to the invention can be used as etching resist, mold for material deposition, and three dimensional objects of device itself. The coating substrates and processing conditions vary accordingly. Such example is described in U.S. Pat. No. 6,391,523.

The compounds of formula I, and the polymers comprising repeating units derived from a compound of the formula I according to the present invention, in combination with a sensitizer compound as described above, can also be used in holographic data storage (HDS) systems as for example described in WO 03/021358.

The compositions according to the invention are also outstandingly suitable as coating compositions for substrates of all types, including wood, textiles, paper, ceramics, glass, plastics, such as polyesters, polyethylene terephthalate, polyolefins or cellulose acetate, especially in the form of films, but especially for coating metals, such as Ni, Fe, Zn, Mg, Co or especially Cu and Al, and also Si, silicon oxides or nitrides, to which an image is to be applied by means of image-wise irradiation.

The invention relates also to the use of compounds of formula I, or the polymers comprising repeating units derived from a compound of the formula I as photolatent acid donors in compositions that can be crosslinked under the action of an acid and/or as dissolution enhancers in compositions wherein the solubility is increased under the action of an acid.

Subject of the invention further is a process of crosslinking compounds that can be crosslinked under the action of an acid, which method comprises adding a compound of formula I, or the polymers comprising repeating units derived from a compound of the formula I to the above-mentioned compositions and irradiating imagewise or over the whole area with light having a wavelength of 10-1500 nm.

The invention relates also to the use of compounds of formulae I, or the polymers comprising repeating units derived from a compound of the formula I as photosensitive acid donors in the preparation of pigmented and non-pigmented surface coatings, adhesives, laminating adhesives, structural adhesives, pressure-sensitive adhesives, printing inks, printing plates, relief printing plates, planographic printing plates, intaglio printing plates, processes printing plates, screen printing stencils, dental compositions, colour filters, spacers, electroluminescence displays and liquid crystal displays (LCD), waveguides, optical switches, color proofing systems, resists, photoresists for electronics, electroplating resists, etch resists both for liquid and dry films, solder resist, photoresist materials for a UV and visible laser direct imaging system, photoresist materials for forming dielectric layers in a sequential build-up layer of a printed circuit board, image-recording materials, image-recording materials for recording holographic images, optical information storage or holographic data storage, decolorizing materials, decolorizing materials for image recording materials, image recording materials using microcapsules, magnetic recording materials, micromechanical parts, plating masks, etch masks, glass fibre cable coatings, microelectronic circuits; in particular to the use of compounds of the formula I; or polymers comprising at least one repeating unit derived from the compound of the formula I and optionally repeating units derived from ethylenically unsaturated compounds selected from the group of formula II, as photosensitive acid donors in the preparation of surface coatings, printing inks, printing plates, dental compositions, colour filters, resists or image-recording materials, or image-recording materials for recording holographic images; as well as to a process for the preparation for the preparation of pigmented and non-pigmented surface coatings, adhesives, laminating adhesives, structural adhesives, pressure-sensitive adhesives, printing inks, printing plates, relief printing plates, planographic printing plates, intaglio printing plates, processes printing plates, screen printing stencils, dental compositions, colour filters, spacers, electroluminescence displays and liquid crystal displays (LCD), waveguides, optical switches, color proofing systems, resists, photoresists for electronics, electroplating resists, etch resists both for liquid and dry films, solder resist, photoresist materials for a UV and visible laser direct imaging system, photoresist materials for forming dielectric layers in a sequential build-up layer of a printed circuit board, image-recording materials, image-recording materials for recording holographic images, optical information storage or holographic data storage, decolorizing materials, decolorizing materials for image recording materials, image recording materials using microcapsules, magnetic recording materials, micromechanical parts, plating masks, etch masks, glass fibre cable coatings, microelectronic circuits; in particular to a process for the preparation of surface coatings, printing inks, printing plates, dental compositions, colour filters, resists, or image-recording materials, or image-recording materials for recording holographic images.

Subject of the invention is also the use of compounds of formula I, or the polymers comprising repeating units derived from a compound of the formula I as photosensitive acid donors in the preparation of colour filters or chemically amplified resist materials; as well as to a process for the preparation of colour filters or chemically amplified resist materials.

The invention further pertains to a color filter prepared by providing red, green and blue picture elements and a black matrix, all comprising a photosensitive resin and a pigment and/or dye on a transparent substrate and providing a transparent electrode either on the surface of the substrate or on the surface of the color filter layer, wherein said photosensitive resin comprises compounds of formula I, or the polymers comprising repeating units derived from a compound of the formula I as photosensitive acid donors.

The person skilled in the art is aware of suitable pigments or dyes to provide the color elements, as well as the black matrix and corresponding suitable resins as shown in, for examples, JP-A-9-203806, JP-A-10-282650, JP-A-10-333334, JP-A-11-194494, JP-A-10-203037, JP-A-2003-5371.

As already mentioned above, in photocrosslinkable compositions, sulfonium salt derivatives act as latent curing catalysts: when irradiated with light they release acid which catalyses the crosslinking reaction. In addition, the acid released by the radiation can, for example, catalyse the removal of suitable acid-sensitive protecting groups from a polymer structure, or the cleavage of polymers containing acid-sensitive groups in the polymer backbone. Other applications are, for example, colour-change systems based on a change in the pH or in the solubility of, for example, a pigment protected by acid-sensitive protecting groups.

Sulfonium salt derivatives according to the present invention can also be used to produce so-called “print-out” images when the compound is used together with a colorant that changes colour when the pH changes, as described e.g. in JP Hei 4 328552-A or in US 5237059. Such color-change systems can be used according to EP 199672 also to monitor goods that are sensitive to heat or radiation.

In addition to a colour change, it is possible during the acid-catalysed deprotection of soluble pigment molecules (as described e.g. in EP 648770, EP 648817 and EP 742255) for the pigment crystals to be precipitated; this can be used in the production of colour filters as described e.g. in EP 654711 or print out images and indicator applications, when the colour of the latent pigment precursor differs from that of the precipitated pigment crystal.

Compositions using pH sensitive dyes or latent pigments in combination with sulfonium salt derivatives can be used as indicators for electromagnetic radiation, such as gamma radiation, electron beams, UV- or visible light, or simple throw away dosimeters. Especially for light, that is invisible to the human eye, like UV- or IR-light, such dosimeters are of interest.

Finally, sulfonium salt derivatives that are sparingly soluble in an aqueous-alkaline developer can be rendered soluble in the developer by means of light-induced conversion into the free acid, with the result that they can be used as solubility enhancers in combination with suitable film-forming resins.

Resins which can be crosslinked by acid catalysis and accordingly by the photolatent acids of formula I, or the polymers comprising repeating units derived from a compound of the formula I according to the invention, are, for example, mixtures of polyfunctional alcohols or hydroxy-group-containing acrylic and polyester resins, or partially hydrolysed polyvinylacetals or polyvinyl alcohols with polyfunctional acetal derivatives. Under certain conditions, for example the acid-catalysed self-condensation of acetal-functionalised resins is also possible.

Suitable acid-curable resins in general are all resins whose curing can be accelerated by acid catalysts, such as aminoplasts or phenolic resole resins. These resins are for example melamine, urea, epoxy, phenolic, acrylic, polyester and alkyd resins, but especially mixtures of acrylic, polyester or alkyd resins with a melamine resin. Also included are modified surface-coating resins, such as acrylic-modified polyester and alkyd resins. Examples of individual types of resins that are covered by the expression acrylic, polyester and alkyd resins are described, for example, in Wagner, Sarx, Lackkunstharze (Munich, 1971), pp. 86-123 and pp. 229-238, or in Ullmann, Encyclopädie der techn. Chemie, 4th Ed., Vol. 15 (1978), pp. 613-628, or Ullmann's Encyclopedia of Industrial Chemistry, Verlag Chemie, 1991, Vol. 18, p. 360 ff., Vol. A19, p. 371 ff.

In coating applications the surface coating preferably comprises an amino resin. Examples thereof are etherified or non-etherified melamine, urea, guanidine or biuret resins. Acid catalysis is especially important in the curing of surface coatings comprising etherified amino resins, such as methylated or butylated melamine resins (N-methoxymethyl- or N-butoxymethyl-melamine) or methylated/butylated glycolurils. Examples of other resin compositions are mixtures of polyfunctional alcohols or hydroxy-group-containing acrylic and polyester resins, or partially hydrolysed polyvinyl acetate or polyvinyl alcohol with polyfunctional dihydropropanyl derivatives, such as derivatives of 3,4-dihydro-2H-pyran-2-carboxylic acid. Polysiloxanes can also be crosslinked using acid catalysis. These siloxane group-containing resins can, for example, either undergo self-condensation by means of acid-catalysed hydrolysis or be crosslinked with a second component of the resin, such as a polyfunctional alcohol, a hydroxy-group-containing acrylic or polyester resin, a partially hydrolysed polyvinyl acetal or a polyvinyl alcohol. This type of polycondensation of polysiloxanes is described, for example, in J. J. Lebrun, H. Pode, Comprehensive Polymer Science, Vol. 5, p. 593, Pergamon Press, Oxford, 1989. Other cationically polymerisable materials that are suitable for the preparation of surface coatings are ethylenically unsaturated compounds polymerisable by a cationic mechanism, such as vinyl ethers, for example methyl vinyl ether, isobutyl vinyl ether, trimethylolpropane trivinyl ether, ethylene glycol divinyl ether; cyclic vinyl ethers, for example 3,4-dihydro-2-formyl-2H-pyran (dimeric acrolein) or the 3,4-dihydro-2H-pyran-2-carboxylic acid ester of 2-hydroxymethyl-3,4-dihydro-2H-pyran; vinyl esters, such as vinyl acetate and vinyl stearate, mono- and di-olefins, such as a-methylstyrene, N-vinylpyrrolidone or N-vinylcarbazole.

For certain purposes, resin mixtures having monomeric or oligomeric constituents containing polymerisable unsaturated groups are used. Such surface coatings can also be cured using compounds of formula I, or the polymers comprising repeating units derived from a compound of the formula I. In that process, radical polymerisation initiators or photoinitiators can additionally be used. The former initiate polymerisation of the unsaturated groups during heat treatment, the latter during UV irradiation.

The invention also relates to a composition comprising

(b) as photosensitive acid donor and as compound whose solubility is increased upon the action of an acid, at least one compound of the formula I and/or a polymer comprising at least one repeating unit derived from a compound of the formula I and repeating units derived from ethylenically unsaturated compounds of formula II.

The invention further pertains to a composition comprising

(a) a compound which cures upon the action of an acid or a compound whose solubility is increased upon the action of an acid; and
(b) as photosensitive acid donor, at least one compound of the formula I and/or a polymer comprising at least one repeating unit derived from a compound of the formula I and repeating units derived from ethylenically unsaturated compounds selected from the group of formula II.

According to the invention, the compounds of formula I, or the polymers comprising repeating units derived from a compound of the formula I can be used together with further photosensitive acid donor compounds (b1), further photoinitiators (d), sensitizers (e) and/or additives (c). Suitable photosensitive acid donor compounds (b1), sensitizers (e) and additives (c) are described above.

Examples of additional photoinitiators (d) are radical photoinitiators, such as for example camphor quinone; benzophenone, benzophenone derivatives; ketal compounds, as for example benzyldimethylketal (IRGACURE® 651); acetophenone, acetophenone derivatives, for example α-hydroxycycloalkyl phenyl ketones or α-hydroxyalkyl phenyl ketones, such as for example 2-hydroxy-2-methyl-1-phenyl-propanone (DAROCUR® 1173), 1-hydroxy-cyclohexyl-phenyl-ketone (IRGACURE® 184), 1-(4-dodecylbenzoyl)-1-hydroxy-1-methyl-ethane, 1-(4-isopropylbenzoyl)-1-hydroxy-1-methyl-ethane, 1-[4-(2-hydroxyethoxy)-phenyl]-2-hydroxy-2-methyl-1-propan-1-one (IRGACURE®2959); 2-hydroxy-1-{4-[4-(2-hydroxy-2-methylpropionyl)-benzyl]-phenyl}-2-methyl-propan-1-one (IRGACURE®127); 2-hydroxy-1-{4-[4-(2-hydroxy-2-methyl-propionyl)-phenoxy]-phenyl}-2-methyl-propan-1-one; dialkoxyacetophenones, α-hydroxy- or α-aminoacetophenones, e.g. (4-methylthiobenzoyl)-1-methyl-1-morpholinoethane (IRGACURE®907), (4-morpholinobenzoyl)-1-benzyl-1-dimethylaminopropane (IRGACURE®379) (4-morpholinobenzoyl)-1-(4-methylbenzyl)-1-dimethylaminopropane (IRGACURE® 369), (4-(2-hydroxyethyl)aminobenzoyl)-1-benzyl-1-dimethylaminopropane), (3,4-dimethoxybenzoyl)-1-benzyl-1-dimethylaminopropane; 4-aroyl-1,3-dioxolanes, benzoin alkyl ethers and benzil ketals, e.g. dimethyl benzil ketal, phenylglyoxalic esters and derivatives thereof, e.g. oxo-phenyl-acetic acid 2-(2-hydroxy-ethoxy)-ethyl ester, dimeric phenylglyoxalic esters, e.g. oxo-phenyl-acetic acid 1-methyl-2-[2-(2-oxo-2-phenyl-acetoxy)-propoxy]-ethyl ester (IRGACURE® 754); oximeesters, e.g. 1,2-octane-dione 1-[4-(phenylthio)phenyl]-2-(O-benzoyloxime) (IRGACURE® OXE01), ethanone 1-[9-ethyl-6-(2-methylbenzoyl)-9H-carbazol-3-yl]-1-(O-acetyloxime) (IRGACURE® OXE02), 9H-thioxanthene-2-carboxaldehyde 9-oxo-2-(O-acetyloxime), peresters, e.g. benzophenone tetracarboxylic peresters as described for example in EP 126541, monoacyl phosphine oxides, e.g. (2,4,6-trimethylbenzoyl)diphenylphosphine oxide (DAROCUR® TPO), ethyl (2,4,6-trimethylbenzoyl phenyl) phosphinic acid ester; bisacylphosphine oxides, e.g. bis(2,6-dimethoxy-benzoyl)-(2,4,4-trimethyl-pentyl)phosphine oxide, bis(2,4,6-trimethylbenzoyl)-phenylphosphine oxide (IRGACURE® 819), bis(2,4,6-trimethylbenzoyl)-2,4-dipentoxy-phenylphosphine oxide, trisacylphosphine oxides, halomethyltriazines, e.g. 2-[2-(4-methoxy-phenyl)-vinyl]-4,6-bis-trichloromethyl-[1,3,5]triazine, 2-(4-methoxy-phenyl)-4,6-bis-trichloromethyl-[1,3,5]triazine, 2-(3,4-dimethoxy-phenyl)-4,6-bis-trichloromethyl-[1,3,5]triazine, 2-methyl-4,6-bis-trichloromethyl-[1,3,5]triazine, hexaarylbisimidazole/coinitiators systems, e.g. ortho-chlorohexaphenyl-bisimidazole combined with 2-mercapto-benzthiazole, ferrocenium compounds, or titanocenes, e.g. bis(cyclopentadienyl)-bis(2,6-difluoro-3-pyrryl-phenyl)titanium (IRGACURE®784). Further, borate compounds, as for example described in U.S. Pat. No. 4,772,530, EP 775706, GB 2307474, GB 2307473 and GB 2304472. The borate compounds preferably are used in combination with electron acceptor compounds, such as, for example dye cations, or thioxanthone derivatives. The DAROCUR® and IRGACURE® compounds are available from Ciba Inc.

Further examples of additional photoinitiators are peroxide compounds, e.g. benzoyl peroxide (other suitable peroxides are described in U.S. Pat. No. 4,950,581, col. 19, I. 17-25) or cationic photoinitiators, such as aromatic sulfonium or iodonium salts, such as those to be found in U.S. Pat. No. 4,950,581, col. 18, I. 60 to col. 19, I. 10, or cyclopentadienyl-arene-iron(II) complex salts, for example (η⁶-isopropylbenzene)(η⁵-cyclopentadienyl)-iron(II) hexafluorophosphate.

The surface coatings may be solutions or dispersions of the surface-coating resin in an organic solvent or in water, but they may also be solventless. Of special interest are surface coatings having a low solvent content, so-called “high solids surface coatings”, and powder coating compositions. The surface coatings may be clear lacquers, as used, for example, in the automobile industry as finishing lacquers for multilayer coatings. They may also comprise pigments and/or fillers, which may be inorganic or organic compounds, and metal powders for metal effect finishes.

The surface coatings may also comprise relatively small amounts of special additives customary in surface-coating technology, for example flow improvers, thixotropic agents, leveling agents, antifoaming agents, wetting agents, adhesion promoters, light stabilisers, antioxidants, or sensitizers.

UV absorbers, such as those of the hydroxyphenyl-benzotriazole, hydroxyphenyl-benzophenone, oxalic acid amide or hydroxyphenyl-s-triazine type may be added to the compositions according to the invention as light stabilisers. Individual compounds or mixtures of those compounds can be used with or without the addition of sterically hindered amines (HALS).

Examples of such UV absorbers and light stabilisers are

1. 2-(2′-Hydroxyphenyl)-benzotriazoles, such as 2-(2′-hydroxy-5′-methylphenyl)-benzotriazole, 2-(3′,5′-di-tert-butyl-2′-hydroxyphenyl)-benzotriazole, 2-(5′-tert-butyl-2′-hydroxyphenyl)-benzotriazole, 2-(2′-hydroxy-5′-(1,1,3,3-tetramethylbutyl)phenyl)-benzotriazole, 2-(3′,5′-di-t-butyl-2′-hydroxyphenyl)-5-chloro-benzotriazole, 2-(3′-tert-butyl-2′-hydroxy-5′-methylphenyl)-5-chloro-benzotriazole, 2-(3′-sec-butyl-5′-tert-butyl-2′-hydroxyphenyl)-benzotriazole, 2-(2′-hydroxy-4′-octyloxyphenyl)-benzotriazole, 2-(3′,5′-di-tert-amyl-2′-hydroxyphenyl)-benzotriazole, 2-(3′,5′-bis-(α,α-dimethylbenzyl)-2′-hydroxyphenyl)-benzotriazole, mixture of 2-(3′-tert-butyl-2′-hydroxy-5′-(2-octyloxycarbonylethyl)phenyl)-5-chloro-benzotriazole, 2-(3′-t-butyl-5′-[2-(2-ethyl-hexyloxy)-carbonylethyl]-2′-hydroxyphenyl)-5-chloro-benzotriazole, 2-(3′-tert-butyl-2′-hydroxy-5′-(2-methoxycarbonylethyl)phenyl)-5-chloro-benzotriazole, 2(3′-tert-butyl-2′-hydroxy-5′-(2-methoxycarbonylethyl)phenyl)-benzotriazole, 2-(3′-tert-butyl-2′-hydroxy-5′-(2-octyloxycarbonylethyl)phenyl)-benzotriazole, 2-(3′-tert-butyl-5′-[2-(2-ethylhexyloxy)carbonylethyl]-2′-hydroxyphenyl)-benzotriazole, 2-(3′-dodecyl-2′-hydroxy-5′-methylphenyl)-benzotriazole and 2-(3′-tert-butyl-2′-hydroxy-5′-(2-isooctyloxycarbonylethyl)phenyl-benzotriazole, 2,2′-methylene-bis[4-(1,1,3,3-tetramethylbutyl)-6-benzotriazol-2-yl-phenol]; transesterification product of 2-[3′-tert-butyl-5′-(2-methoxycarbonylethyl)-2′-hydroxyphenyl]-benzotriazole with polyethylene glycol 300; [R—CH₂CH₂—COO(CH₂)₃]₂— wherein R=3′-tert-butyl-4′-hydroxy-5′-2 H-benzotriazol-2-yl-phenyl.
2. 2-Hydroxybenzophenones, such as the 4-hydroxy, 4-methoxy, 4-octyloxy, 4-decyloxy, 4-dodecyloxy, 4-benzyloxy, 4,2′,4′-trihydroxy or 2′-hydroxy-4,4′-dimethoxy derivative.
3. Esters of unsubstituted or substituted benzoic acids, such as 4-tert-butyl-phenyl salicylate, phenyl salicylate, octylphenyl salicylate, dibenzoylresorcinol, bis(4-tert-butylbenzoyl)resorcinol, benzoylresorcinol, 3,5-di-tert-butyl-4-hydroxybenzoic acid 2,4-di-tert-butylphenyl ester, 3,5-di-tert-butyl-4-hydroxybenzoic acid hexadecyl ester, 3,5-di-tert-butyl-4-hydroxybenzoic acid octadecyl ester, 3,5-di-tert-butyl-4-hydroxybenzoic acid 2-methyl-4,6-di-tert-butylphenyl ester.
4. Acrylates, such as α-cyano-β,β-diphenylacrylic acid ethyl ester or isooctyl ester, α-carbomethoxy-cinnamic acid methyl ester, α-cyano-β-methyl-p-methoxy-cinnamic acid methyl ester or butyl ester, α-carbomethoxy-p-methoxy-cinnamic acid methyl ester, N-(b-carbomethoxy-β-cyanovinyl)-2-methyl-indoline.
5. Sterically hindered amines, such as bis(2,2,6,6-tetramethyl-piperidyl)sebacate, bis(2,2,6,-6-tetramethyl-piperidyl)succinate, bis(1,2,2,6,6-pentamethylpiperidyl)sebacate, n-butyl-3,-5-di-tert-butyl-4-hydroxybenzyl-malonic acid bis(1,2,2,6,6-pentamethylpiperidyl) ester, condensation product of 1-hydroxyethyl-2,2,6,6-tetramethyl-4-hydroxypiperidine and succinic acid, condensation product of N,N′-bis(2,2,6,6-tetramethyl-4-piperidyl)hexamethylenediamine and 4-tert-octylamino-2,6-dichloro-1,3,5-s-triazine, tris(2,2,6,6-tetramethyl-4-piperidyl)nitrilo-triacetate, tetrakis(2,2,6,6-tetramethyl-4-piperidyl)-1,2,3,4-butanetetraoate, 1,1′-(1,2-ethanediyl)-bis(3,3,5,5-tetramethyl-piperazinone), 4-benzoyl-2,2,6,6-tetramethylpiperidine, 4-stearyl-oxy-2,2,6,6-tetramethylpiperidine, bis(1,2,2,6,6-pentamethylpiperidyl)-2-n-butyl-2-(2-hydroxy-3,5-di-tert-butylbenzyl) malonate, 3-n-octyl-7,7,9,9-tetramethyl-1,3,8-triazaspiro[4.5]decane-2,4-dione, bis(1-octyloxy-2,2,6,6-tetramethylpiperidyl)sebacate, bis(1-octyloxy-2,2,6,6-tetramethylpiperidyl)succinate, condensation product of N,N′-bis(2,2,6,6-tetra-methyl-4-piperidyl)-hexamethylenediamine and 4-morpholino-2,6-dichloro-1,3,5-triazine, condensation product of 2-chloro-4,6-di(4-n-butylamino-2,2,6,6-tetramethylpiperidyl)-1,3,5-triazine and 1,2-bis(3-aminopropylamino)ethane, condensation product of 2-chloro-4,6-di(4-n-butylamino-1,2,2,6,6-pentamethylpiperidyl)-1,3,5-triazine and 1,2-bis(3-aminopropylamino)ethane, 8-acetyl-3-dodecyl-7,7,9,9-tetramethyl-1,3,8-triazaspiro[4.5]decane-2,4-dione, 3-dodecyl-1-(2,2,6,6-tetramethyl-4-piperidyl)pyrrolidine-2,5-dione, 3-dodecyl-1-(1,2,2,6,6-pentamethyl-4-piperidyl)-pyrrolidine-2,5-dione.
6. Oxalic acid diamides, such as 4,4′-dioctyloxy-oxanilide, 2,2′-diethoxy-oxanilide, 2,2′-di-octyloxy-5,5′-di-tert-butyl-oxanilide, 2,2′-didodecyloxy-5,5′-di-tert-butyl-oxanilide, 2-ethoxy-2′-ethyl-oxanilide, N,N′-bis(3-dimethylaminopropyl)oxalamide, 2-ethoxy-5-tert-butyl-2′-ethyloxanilide and a mixture thereof with 2-ethoxy-2′-ethyl-5,4′-di-tert-butyl-oxanilide, mixtures of o- and p-methoxy- and of o- and p-ethoxy-di-substituted oxanilides.
7. 2-(2-Hydroxyphenyl)-1,3,5-triazines, such as 2,4,6-tris(2-hydroxy-4-octyloxyphenyl)-1,3,5-triazine, 2-(2-hydroxy-4-octyloxyphenyl)-4,6-bis(2,4-dimethylphenyl)-1,3,5-triazine, 2-(2,4-dihydroxyphenyl)-4,6-bis(2,4-dimethylphenyl)-1,3,5-triazine, 2,4-bis(2-hydroxy-4-propyloxyphenyl)-6-(2,4-dimethylphenyl)-1,3,5-triazine, 2-(2-hydroxy-4-octyloxyphenyl)-4,6-bis(4-methylphenyl)-1,3,5-triazine, 2-(2-hydroxy-4-dodecyloxyphenyl)-4,6-bis(2,4-dimethylphenyl)-1,3,5-triazine, 2-[2-hydroxy-4-(2-hydroxy-3-butyloxy-propyloxy)phenyl]-4,6-bis(2,4-dimethyl-phenyl)-1,3,5-triazine, 2-[2-hydroxy-4-(2-hydroxy-3-octyloxy-propyloxy)phenyl]-4,6-bis(2,4-dimethylphenyl)-1,3,5-triazine, 2-[4-dodecyl-/tridecyl-oxy-(2-hydroxypropyl)oxy-2-hydroxyphenyl]-4,6-bis(2,4-dimethylphenyl)-1,3,5-triazine.
8. Phosphites and phosphonites, such as triphenyl phosphite, diphenyl alkyl phosphites, phenyl dialkyl phosphites, tris(nonylphenyl) phosphite, trilauryl phosphite, trioctadecyl phosphite, distearyl-pentaerythritol diphosphite, tris(2,4-di-tert-butylphenyl) phosphite, diisodecyl-pentaerythritol diphosphite, bis(2,4-di-tert-butylphenyl)pentaerythritol diphosphite, bis(2,6-di-tertbutyl-4-methylphenyl)pentaerythritol diphosphite, bis-isodecyloxy-pentaerythritol diphosphite, bis(2,4-di-tert-butyl-6-methylphenyl)pentaerythritol diphosphite, bis-(2,4,6-tri-tert-butylphenyl)pentaerythritol diphosphite, tristearyl-sorbitol triphosphate, tetrakis(2,4-di-tert-butylphenyl)-4,4′-biphenylene diphosphonate, 6-isooctyloxy-2,4,8,10-tetra-tert-butyl-12H-dibenzo-[d,g]-1,3,2-dioxaphosphocine, 6-fluoro-2,4,8,10-tetra-tert-butyl-12-methyl-dibenzo[d,g]-1,3,-2-dioxaphosphocine, bis(2,4-di-tert-butyl-6-methylphenyl)methyl phosphite, bis(2,4-di-tert-butyl-6-methylphenyl)ethyl phosphite.

Such light stabilisers can also be added, for example, to an adjacent surface-coating layer from which they gradually diffuse into the layer of stoving lacquer to be protected. The adjacent surface-coating layer may be a primer under the stoving lacquer or a finishing lacquer over the stoving lacquer.

It is also possible to add to the resin, for example, photosensitisers which shift or increase the spectral sensitivity so that the irradiation period can be reduced and/or other light sources can be used. Examples of photosensitisers are aromatic ketones or aromatic aldehydes (as described, for example, in U.S. Pat. No. 4,017,652), 3-acyl-coumarins (as described, for example, in U.S. Pat. No. 4,366,228, EP 738928, EP 22188), keto-coumarins (as described e.g. in U.S. Pat. No. 5,534,633, EP 538997, JP 8272095-A), styryl-coumarins (as described e.g. in EP 624580), 3-(aroylmethylene)-thiazolines, thioxanthones, condensed aromatic compounds, such as perylene, aromatic amines (as described, for example, in U.S. Pat. No. 4,069,954 or WO 96/41237) or cationic and basic colourants (as described, for example, in U.S. Pat. No. 4,026,705), for example eosine, rhodanine and erythrosine colourants, as well as dyes and pigments as described for example in JP 8320551-A, EP 747771, JP 7036179-A, EP 619520, JP 6161109-A, JP 6043641, JP 6035198-A, WO 93/15440, EP 568993, JP 5005005-A, JP 5027432-A, JP 5301910-A, JP 4014083-A, JP 4294148-A, EP 359431, EP 103294, U.S. Pat. No. 4,282,309, EP 39025, EP 5274, EP 727713, EP 726497 or DE 2027467.

Other customary additives are—depending on the intended use—optical brighteners, fillers, pigments, colourants, wetting agents or flow improvers and adhesion promoters.

For curing thick and pigmented coatings, the addition of micro glass beads or powdered glass fibres, as described in U.S. Pat. No. 5,013,768, is suitable.

Sulfonium salt derivatives can also be used, for example, in hybrid systems. These systems are based on formulations that are fully cured by two different reaction mechanisms. Examples thereof are systems that comprise components that are capable of undergoing an acid-catalysed crosslinking reaction or polymerisation reaction, but that also comprise further components that crosslink by a second mechanism. Examples of the second mechanism are radical full cure, oxidative crosslinking or humidity-initiated crosslinking. The second curing mechanism may be initiated purely thermally, if necessary with a suitable catalyst, or also by means of light using a second photoinitiator. Suitable additional photoinitiators are described above.

If the composition comprises a radically crosslinkable component, the curing process, especially of compositions that are pigmented (for example with titanium dioxide), can also be assisted by the addition of a component that is radical-forming under thermal conditions, such as an azo compound, for example 2, 2′-azobis(4-methoxy-2,4-dimethylvaleronitrile), a triazene, a diazosulfide, a pentazadiene or a peroxy compound, such as, for example, a hydroperoxide or peroxycarbonate, for example tert-butyl hydroperoxide, as described, for example, in EP 245639. The addition of redox initiators, such as cobalt salts, enables the curing to be assisted by oxidative crosslinking with oxygen from the air.

The surface coating can be applied by one of the methods customary in the art, for example by spraying, painting or immersion. When suitable surface coatings are used, electrical application, for example by anodic electrophoretic deposition, is also possible. After drying, the surface coating film is irradiated. If necessary, the surface coating film is then fully cured by means of heat treatment.

The compounds of formula I, or the polymers comprising repeating units derived from a compound of the formula I can also be used for curing moldings made from composites. A composite consists of a self-supporting matrix material, for example a glass fibre fabric, impregnated with the photocuring formulation.

It is known from EP 592139 that sulfonate derivatives can be used as acid generators, which can be activated by light in compositions that are suitable for the surface treatment and cleaning of glass, aluminium and steel surfaces. The use of such compounds in organosilane systems results in compositions that have significantly better storage stability than those obtained when the free acid is used. The compounds of formula I, or the polymers comprising repeating units derived from a compound of the formula I are also suitable for this application.

The sulfonium salt derivatives of the present invention can also be used to shape polymers that undergo an acid induced transition into a state where they have the required properties using photolithography. For instance the sulfonium salt derivatives can be used to pattern conjugated emissive polymers as described, for example, in M. L. Renak; C. Bazan; D. Roitman; Advanced materials 1997, 9, 392. Such patterned emissive polymers can be used to manufacture microscalar patterned Light Emitting Diodes (LED) which can be used to manufacture displays and data storage media. In a similar way precursors for polyimides (e.g. polyimide precursors with acid labile protecting groups that change solubility in the developer) can be irradiated to form patterned polyimide layers which can serve as protective coatings, insulating layers and buffer layers in the production of microchips and printed circuit boards.

The formulations of the invention may also be used as conformal coatings, photoimagable insulating layers and dielectrics as they are used in sequential build up systems for printed circuit boards, stress buffer layers in the manufacturing of integrated circuits.

It is known that conjugated polymers like, e.g. polyanilines can be converted from semiconductive to conductive state by means of proton doping. The sulfonium salt derivatives of the present invention can also be used to imagewise irradiate compositions comprising such conjugated polymers in order to form conducting structures (exposed areas) embedded in insulating material (non exposed areas). These materials can be used as wiring and connecting parts for the production of electric and electronic devices.

Suitable radiation sources for the compositions comprising compounds of formula I, or the polymers comprising repeating units derived from a compound of the formula I are radiation sources that emit radiation of a wavelength of approximately from 10 to 1500, for example from 10 to 1000, or preferably from 10 to 700 nanometers as well as e-beam radiation and high-energy electromagnetic radiation such as X-rays. Both, point sources and platform projectors (lamp carpets) are suitable. Examples are: carbon arc lamps, xenon arc lamps, medium pressure, high pressure and low pressure mercury lamps, optionally doped with metal halides (metal halide lamps), microwave-excited metal vapour lamps, excimer lamps, superactinic fluorescent tubes, fluorescent lamps, argon filament lamps, electronic flash lamps, photographic flood lights, electron beams and X-ray beams generated by means of synchrotrons or laser plasma. The distance between the radiation source and the substrate according to the invention to be irradiated can vary, for example, from 2 cm to 150 cm, according to the intended use and the type and/or strength of the radiation source. Suitable radiation sources are especially mercury vapour lamps, especially medium and high pressure mercury lamps, from the radiation of which emission lines at other wavelengths can, if desired, be filtered out. That is especially the case for relatively short wavelength radiation. It is, however, also possible to use low energy lamps (for example fluorescent tubes) that are capable of emitting in the appropriate wavelength range. An example thereof is the Philips TL03 lamp. Another type of radiation source that can be used are the light emitting diodes (LED) that emit at different wavelengths throughout the whole spectrum either as small band emitting source or as broad band (white light) source. Also suitable are laser radiation sources, for example excimer lasers, such as Kr-F lasers for irradiation at 248 nm, Ar-F lasers at 193 nm, or F₂ laser at 157 nm. Lasers in the visible range and in the infrared range can also be used. Especially suitable is radiation of the mercury i, h and g lines at wavelengths of 365, 405 and 436 nanometers. As a light source further EUV (Extreme Ultra Violet) at 13 nm is also suitable. A suitable laser-beam source is, for example, the argon-ion laser, which emits radiation at wavelengths of 454, 458, 466, 472, 478, 488 and 514 nanometers. Nd-YAG-lasers emitting light at 1064 nm and its second and third harmonic (532 nm and 355 nm respectively) can also be used. Also suitable is, for example, a helium/cadmium laser having an emission at 442 nm or lasers that emit in the UV range. With that type of irradiation, it is not absolutely essential to use a photomask in contact with the photopolymeric coating to produce a positive or negative resist; the controlled laser beam is capable of writing directly onto the coating. For that purpose the high sensitivity of the materials according to the invention is very advantageous, allowing high writing speeds at relatively low intensities. On irradiation, the sulfonium salt derivatives in the composition in the irradiated sections of the surface coating decompose to form the acids.

In contrast to customary UV curing with high-intensity radiation, with the compounds according to the invention activation is achieved under the action of radiation of relatively low intensity. Such radiation includes, for example, daylight (sunlight), and radiation sources equivalent to daylight. Sunlight differs in spectral composition and intensity from the light of the artificial radiation sources customarily used in UV curing. The absorption characteristics of the compounds according to the invention are as well suitable for exploiting sunlight as a natural source of radiation for curing. Daylight-equivalent artificial light sources that can be used to activate the compounds according to the invention are to be understood as being projectors of low intensity, such as certain fluorescent lamps, for example the Philips TL05 special fluorescent lamp or the Philips TL09 special fluorescent lamp. Lamps having a high daylight content and daylight itself are especially capable of curing the surface of a surface-coating layer satisfactorily in a tack-free manner. In that case expensive curing apparatus is superfluous and the compositions can be used especially for exterior finishes. Curing with daylight or daylight-equivalent light sources is an energy-saving method and prevents emissions of volatile organic components in exterior applications. In contrast to the conveyor belt method, which is suitable for flat components, daylight curing can also be used for exterior finishes on static or fixed articles and structures.

The surface coating to be cured can be exposed directly to sunlight or daylight-equivalent light sources. The curing can, however, also take place behind a transparent layer (e.g. a pane of glass or a sheet of plastics).

The examples, which follow, illustrate the invention in more detail. Parts and percentages are, as in the remainder of the description and in the claims, by weight, unless stated otherwise. Where alkyl radicals having more than three carbon atoms are referred to without any mention of specific isomers, the n-isomers are meant in each case.

### EXAMPLE 1

### Preparation of

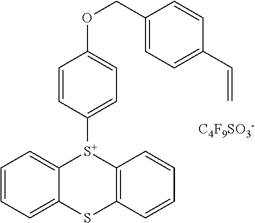

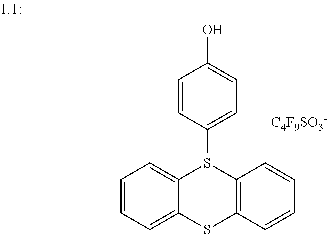

6.12 g of methanesulfonic acid are added to 625 mg of phosphorus oxide, and the mixture is stirred for 1 hour until the phosphorus oxide is dissolved. To the solution, 1.12 g of phenol are added at room temperature, and the reaction mixture is stirred for 30 min. 2.02 g of thianthrene-9-oxide is added. The reaction mixture is stirred overnight at room temperature, and then is poured into an aqueous solution of potassium nonaflate (4.05 g/80 ml). The product is extracted with dichloromethane, and the organic layer is washed with brine and dried over anhydrous magnesium sulfate. After removal of the magnesium sulfate by filtration, the organic extracts are condensed to give a beige solid. The solid is dissolved in a mixture of dichloromethane-methanol (10:1), and purification by column chromatography eluting mixture of dichloromethane-methanol (12:1) as a solvent to give 3.43 g of the compound of example 1.1 as an off-white powder. The structure is confirmed by the ¹H-NMR. δ [ppm]: (DMSO-d₆). δ [ppm]: 6.88 (d, 2H), 7.14 (d, 2H), 7.76 (td, 2H), 7.84 (td, 2H), 8.05 (dd, 2H), 8.39 (dd, 2H), 10.70 (br, 1H).

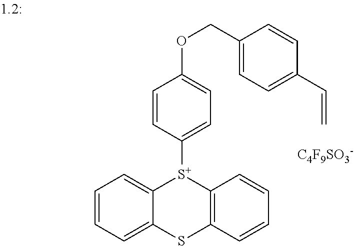

3.08 g of the compound of example 1.1 are dissolved in 20 ml of acetone, and 932 mg of potassium carbonate are added to the solution. After the reaction mixture is stirred for 1 hour at room temperature, 915 mg of 4-vinyl benzylchloride are added, and the reaction mixture is refluxed overnight. After it is cooled to room temperature, the solid is removed by filtration, and the filtrate is condensed to give a brown resin. It is dissolved in 20 ml of THF (tetrahydrofurane), and the solution is added to 400 ml of n-hexane to give 3.22 g of the compound of example 1.2 as a white powder. The structure is confirmed by the ¹H-NMR and ¹⁹F-NMR spectrum (DMSO-d₆). δ [ppm]: 5.07 (s, 2H), 5.22 (dd, 1H), 5.78 (dd, 1H), 6.66 (dd, 1H), 7.12-7.22 (m, 4H), 7.32 (d, 2H), 7.42 (d, 2H), 7.78 (td, 2H), 7.85 (td, 2H), 8.02 (dd, 2H), 8.46 (dd, 2H), −81.41 (t, 3F), −115.04 (t, 2F), −122.22 (s, 2F), −126.24 (d, 2F)

### EXAMPLE 2

### Preparation of

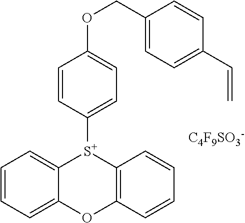

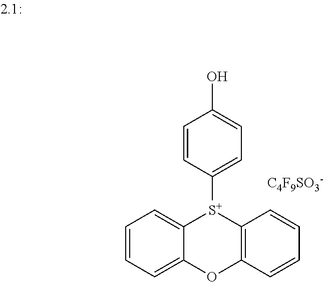

7.39 g of methanesulfonic acid are added to 761 mg of phosphorus oxide, and the mixture is stirred for 1 hour until the phosphorus oxide is dissolved. To the solution, 1.36 g of phenol are added at room temperature, and the reaction mixture is stirred for 30 min. 2.595 g of phenoxthin-10-oxide are added, and after the reaction mixture is stirred overnight at room temperature, it is poured into an aqueous solution of potassium nonaflate (4.86 g/80 ml). The product is extracted with dichloromethane, and the organic layer is washed with brine and dried over anhydrous magnesium sulfate. After removal of magnesium sulfate by filtration, the organic extracts are condensed to give a beige solid. The solid is dissolved in a mixture of dichloromethane-methanol (10:1), and purification by column chromatography eluting mixture of dichloromethane-methanol (12:1) as a solvent to give 6.61 g of the compound of example 2.1 as a white powder. The structure is confirmed by the ¹H-NMR. δ [ppm]: (CDCl₃). δ [ppm]: 6.90 (dd, 2H), 7.38-7.44 (m, 4H), 7.56 (dd, 2H), 7.76 (td, 2H), 7.90 (dd, 2H).

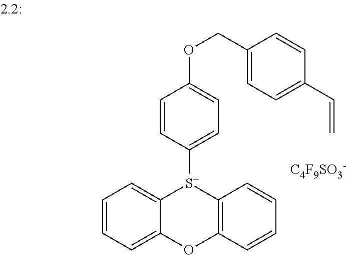

2.96 g of the compound of example 2.1 is dissolved in 20 ml of acetone, and 929 mg of potassium carbonate are added to the solution. After the reaction mixture is stirred for 1 hour at room temperature, 913 mg of 4-vinyl benzylchloride are added, and the reaction mixture is refluxed overnight. After it is cooled to room temperature, the solid is removed by filtration, and the filtrate is condensed to give a brown resin. It is dissolved in 20 ml of THF, and the solution is added to 400 ml of n-hexane to give 3.17 g of the compound of example 2.2 as a white powder. The structure is confirmed by the ¹H-NMR and ¹⁹F-NMR spectrum (DMSO-d₆). δ [ppm]: 5.12 (s, 2H), 5.23 (dd, 1H), 5.80 (dd, 1H), 6.68 (dd, 1H), 7.19 (dd, 4H), 7.35 (d, 2H), 7.42 (d, 2H), 7.49 (td, 2H), 7.66 (d, 2H), 7.83 (td, 2H), 8.18 (dd, 2H), −81.41 (t, 3F), −115.04 (t, 2F), −121.82 (s, 2F), −125.66 (d, 2F).

### EXAMPLE 3

### AcOSty/EAMA/Example 1=11:5:1

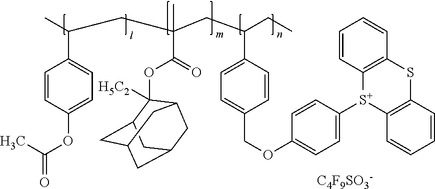

9.73 g of 4-Acetoxystyrene (AcOSty), 9.72 g of 2-ethyl-2-adamantylmethacrylate (EAMA), 3.62 g of the compound of example 1 (PAG), and 1.21 g of V601 produced by Wako Pure Chemical Industries, Ltd., as an initiator are dissolved in 120 ml of 2-butanone. After nitrogen gas is introduced to the system for 20 min, the solution is refluxed for 15 hours. The reaction mixture is poured into 2.4 L of n-hexane to give a white solid. The obtained powder is dissolved in 50 ml of THF, and the solution is poured into a mixed solution of 400 ml of water and 400 ml of methanol to give a white solid. The solid is collected by filtration, and dried at reduced pressure to give 9.87 g of the polymer of example 2. By GPC measurement using polystyrene standard, the weight average molecular weight (Mw) and the number average molecular weight (Mn) of the polymer obtained are 3650 and 2720, respectively.

### EXAMPLE 4

### HOSty/EAMA/Example 1=14:7:1

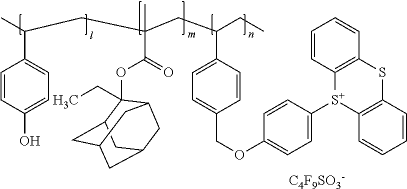

1.02 g of the polymer of example 3 is dissolved in 8 ml of acetonitrile, and then 1.54 g of ammonium acetate is added. The mixture is refluxed overnight. After cooling to room temperature, the reaction mixture is poured into potassium nonaflate aqueous solution (1.62 g/100 ml) to give a white powder, which is collected by filtration. The powder is dried at reduced pressure to give 520 mg of the polymer of example 4. By GPC measurement using polystyrene standard, the weight average molecular weight (Mw) and the number average molecular weight (Mn) of the polymer obtained are 3640 and 2750, respectively.

### EXAMPLE 5

### HOSty/EAMA/Example 2=10:7:1

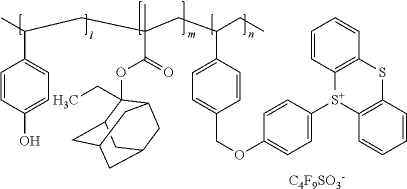

The compound of example 5 is prepared according to the methods as described in examples 3 and 4, by employing the unsaturated sulfonium compound of example 2 instead of the one of example 1.

### Application Examples

### Example A1

### EUV lithography

Positive type photoresist compositions are prepared by mixing and dissolving the components shown in Table 1. Each of the positive tone photoresist compositions is evaluated in lithography properties by forming the resist patterns with the procedure disclosed below.

TABLE 1

| | Components: | | |
|---|---|---|---|
| | Photoacid | | |
| | generator | Additive | Solvent |
| Formulation 1 | Example 4 | (c)-1 | (s)-1 |
| | [100] | [0.2] | [3000] |

(c)-1: triethanolamine
(s)-1: propylene glycol methylether acetate (PGMEA)
numbers in [ ] indicate the amount of the components as parts by weight

A four inches semiconductor silicon wafer is treated with tetramethyldisilazane (TMDS) to improve the wafer's surface hydrophobic properties. On the coating, the positive tone resist compositions are coated by using a spinner followed by drying and post applied baking (PAB) treatment for 60 seconds on a hot plate at 110° C. to form a photoresist layer having a thickness of 60 nm. The photoresist layer is pattern-wise exposed to Extreme Ultraviolet Light of 13.5 nm wavelength with The Swiss Light Source at the Paul Scherrer Institute through a pinhole spatial filter to obtain a spatially coherent and uniform illumination of a mask pattern. Then the layer is post exposure baked (PEB) for 60 seconds on a hot plate at 110° C., developed at 23° C. for 60 seconds with a 2.38% by weight aqueous solution of tetramethylammonium hydroxide followed by rinse with water for 30 seconds and drying to form the resist patters.

### Sensitivity

The optimized exposure dose for the L/S pattern (line width: 50 nm, pitch: 100 nm) to form a resist pattern of 1:1 line and space (L/S pattern, is determined (photosensitivity: Eₒₚ, mJ/cm²). The lower the value, the more sensitive is the resist formulation.

### Resolution

The finest feature size of the photoresist is determined by changing the size of the mask pattern in the previous Eₒₚ determination.

The lower the value, the better is the resolution of the resist formulation.

The results are collected in table 2 below.

TABLE 2

| | E_{OP} | Resolution |
|---|---|---|
| | (mJ/cm²) | (nm) |
| Formulation 1 | 16.3 | 45 |

Obviously from the results shown above, it is confirmed that a hyperfine resist pattern can be formed with the positive tone photoresist composition of the present invention.

### Example A2

### Sensitivity Measurement by EB Exposure and DUV

Positive type photoresist compositions are prepared by mixing and dissolving the components shown in Table 3.

TABLE 3

| | Components: | | |
|---|---|---|---|
| | Photoacid | | |
| | generator | Additive | Solvent |
| Formulation 2 | Example 4 | (c)-1 | (s)-1 |
| | [100] | [0.2] | [1700] |

(c)-1: triethanolamine
(s)-1: PGMEA
numbers in [ ] indicate the amount of the components as parts by weight

The same procedure as described above in example A1 is repeated except that resist thickness is 120 nm instead of 60 nm, that 120° C. for PAB/PEB is applied instead of 110° C. and that a flood exposure by electron beam (EB) with Hitachi JBX-5000SI and by DUV light through a band-pass filter of 254 nm with a Canon mask aligner PLA 521 FA instead of Extreme Ultraviolet light is applied.

### Sensitivity

The minimum exposure dose to clear (E_{O}: μC/cm² or mJ/cm²) is used as a measure for sensitivity. The lower the value, the more sensitive is the resist formulation.

The results are summarized in Table 4.

TABLE 4

| | E_{O} by EB | E_{O} by DUV |
|---|---|---|
| | (μC/cm²) | (mJ/cm²) |
| Formulation 2 | 2 | 1.7 |

1. A compound of the formula I 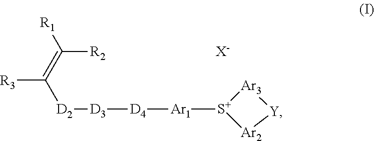
wherein
R₁, R₂ and R₃ independently of each other are hydrogen or C₁-C₁₈alkyl;
Y is O or S;
D₂, D₃ and D₄ independently of each other are O, O(CO), C₁-C₁₈alkylene or Ar₁;
Ar₁ is phenylene,
Ar₂ and Ar₃ are phenylene;
X⁻ is 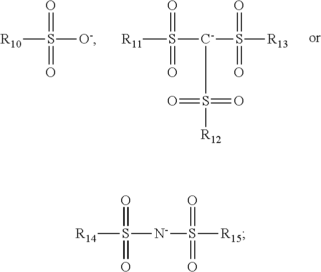
R₁₀ is C₁-C₁₀haloalkyl;
R₁₁, R₁₂ and R₁₃ independently of each other are C₁-C₁₀haloalkyl; and
R₁₄ and R₁₅ independently of each other are C₁-C₁₀haloalkyl;
or R₁₄ and R₁₅, together with —SO₂—N⁻—SO₂— to which they are attached, form a 5-, 6- or 7-membered ring which is substituted by one or more halogen.

2. A polymer comprising at least one repeating unit derived from the compound of the formula I according to claim 1.

3. A polymer according to claim 2 additionally to the at least one repeating unit derived from the compound of the formula I according to claim 1 comprising one or more identical or different repeating units derived from ethylenically unsaturated compounds of formula II 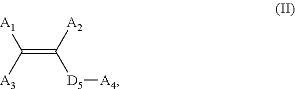
wherein
A₁, A₂ and A₃ independently of each other are hydrogen, halogen, CN, C₁-C₁₈alkyl, C₁-C₁₀haloalkyl, (CO)R₈, (CO)OR₄, (CO)NR₅R₆ or C₁-C₁₈alkyl which is substituted by OR₄;
A₄ is C₁-C₁₈alkyl, C₂-C₁₈alkyl which is interrupted by one or more O, S, NR₇, CO, SO and/or SO₂, C₃-C₃₀cycloalkyl, C₃-C₃₀cycloalkyl which is interrupted by one or more O, S, NR₇, CO, SO and/or SO₂, C₂-C₁₂alkenyl, C₂-C₁₂alkenyl which is interrupted by one or more O, S, NR₇, CO, SO and/or SO₂, C₄-C₃₀cycloalkenyl, C₄-C₃₀cycloalkenyl which is interrupted by one or more O, S, NR₇, CO, SO and/or SO₂,
wherein the groups C₁-C₁₈alkyl, interrupted C₂-C₁₈alkyl, C₃-C₃₀cycloalkyl, interrupted C₃-C₃₀cycloalkyl, C₂-C₁₂alkenyl, interrupted C₂-C₁₂alkenyl, C₄-C₃₀cycloalkenyl and interrupted C₄-C₃₀cycloalkenyl optionally are substituted by one or more Ar, OR₄, (CO)OR₄, O(CO)R₈, halogen, NO₂, CN, NR₅R₆, C₁-C₁₂alkylthio, C₁-C₁₈alkylsulfonyloxy, phenylsulfonyloxy, and/or (4-methylphenyl)sulfonyloxy;
or A₄ is hydrogen, halogen, NO₂, CN, Ar, (CO)R₈, (CO)OR₄, (CO)NR₅R₆, O(CO)R₈, O(CO)OR₄, O(CO)NR₅R₆, NR₇(CO)R₈, NR₇(CO)OR₄, OR₄, NR₅R₆, SR₇, SOR₈, SO₂R₈ and/or OSO₂R₈;
D₅ is a direct bond, O, CO, (CO)O, (CO)S, (CO)NR₇, SO₂, OSO₂, C₁-C₁₈alkylene or Ar₁;
optionally A₃ and D₅, together with the ethylenically unsaturated double bond to which they are attached form C₃-C₃₀cycloalkenyl which optionally is interrupted by one or more O, S, NR₇, CO, SO and/or SO₂;
or optionally the radicals A₂ and D₅ together with the carbon atom of the ethylenically unsaturated double bond to which they are attached form C₃-C₃₀cycloalkyl which optionally is interrupted by one or more O, S, N, NR₇, CO, SO and/or SO₂;
Ar₁ is phenylene, biphenylene, naphthylene, 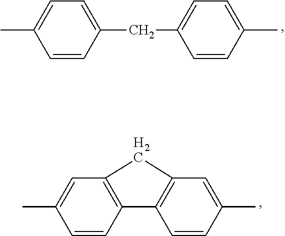
heteroarylene, oxydiphenylene or 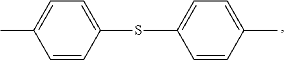
all of which are optionally substituted by one or more C₃-C₃₀cycloalkyl, C₁-C₁₈alkyl, C₁-C₁₀haloalkyl, C₂-C₁₂alkenyl, C₄-C₃₀cycloalkenyl, phenyl-C₁-C₃-alkyl,
or are substituted by C₂-C₁₈alkyl which is interrupted by one or more O, S, NR₇, O(CO), (CO)O, (CO)NR₇ and/or NR₇(CO);
or are substituted by C₃-C₃₀cycloalkyl which is interrupted by one or more O, S, NR₇, O(CO), (CO)O, (CO)NR₇ and/or NR₇(CO);
or are substituted by C₄-C₃₀cycloalkenyl which is interrupted by one or more O, S, NR₇, O(CO), (CO)O, (CO)NR₇ and/or NR₇(CO);
or are substituted by halogen, NO₂, CN, Ar, (CO)R₈, (CO)OR₄, (CO)NR₅R₆, O(CO)R₈, O(CO)OR₄, O(CO)NR₅R₆, NR₇(CO)R₈, NR₇(CO)OR₄, OR₄, NR₅R₆, SR₇, SOR₈, SO₂R₈ and/or —OSO₂R₈,
wherein optionally the substituents C₁-C₁₈alkyl, C₂-C₁₂alkenyl, (CO)R₈, (CO)OR₄, (CO)NR₅R₆, O(CO)R₈, O(CO)OR₄, O(CO)NR₅R₆, NR₇(CO)R₈, NR₇(CO)OR₄, OR₄, NR₅R₆, SR₇, SOR₈, SO₂R₈
and/or OSO₂R₈ form 5-, 6- or 7-membered rings, via the radicals C₁-C₁₈alkyl, C₂-C₁₂alkenyl, R₄, R₅, R₆, R₇ and/or R₈, with further substituents on the phenylene, biphenylene, naphthylene, 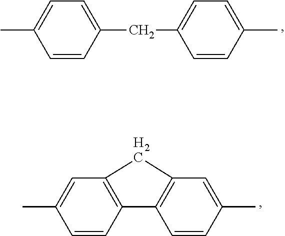
heteroarylene, oxydiphenylene or 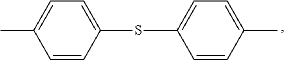
or with one of the carbon atoms of the phenylene, biphenylene, naphthylene, 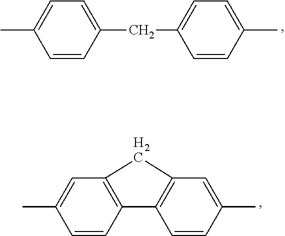
heteroarylene, oxydiphenylene or 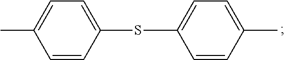
wherein all Ar₁ optionally additionally are substituted by a group having a —O—C-bond or a —O—Si-bond which cleaves upon the action of an acid;
R₄ is hydrogen, C₃-C₃₀cycloalkyl, C₁-C₁₀haloalkyl, C₂-C₁₂alkenyl, O₄—C₃₀cycloalkenyl, phenyl-C₁-C₃-alkyl;
or is C₂-C₁₈alkyl which is interrupted by one or more O, S, NR₇, O(CO), (CO)O, (CO)NR₇ and/or NR₇(CO);
or is C₃-C₃₀cycloalkyl which is interrupted by one or more O, S, NR₇, O(CO), (CO)O, (CO)NR₇ and/or NR₇(CO);
or is C₄-C₃₀cycloalkenyl which is interrupted by one or more O, S, NR₇, O(CO), (CO)O, (CO)NR₇ and/or NR₇(CO);
or R₄ is Ar, (CO)R₈, (CO)OR₈, (CO)NR₅R₆, and/or SO₂R₈;
wherein R₄ as C₃-C₃₀cycloalkyl, C₁-C₁₈alkyl, C₁-C₁₀haloalkyl, C₂-C₁₂alkenyl, C₄-C₃₀cycloalkenyl, phenyl-C₁-C₃-alkyl, interrupted C₂-C₁₈alkyl, interrupted C₃-C₃₀cycloalkyl, interrupted C₄-C₃₀cycloalkenyl and Ar optionally is substituted by one or more Ar, OH, C₁-C₁₈alkyl, C₁-C₁₀haloalkyl, phenyl-C₁-C₃-alkyl, C₃-C₃₀cycloalkyl, halogen, NO₂, CN, C₁-C₁₈alkoxy, phenoxy, phenoxycarbonyl, phenylthio, phenylthiocarbonyl, NR₅R₆, C₁-C₁₂alkoxycarbonyl, C₂-C₁₈alkoxycarbonyl, C₂-C₁₀haloalkanoyl, halobenzoyl, C₁-C₁₈alkylsulfonyl, phenylsulfonyl, (4-methylphenyl)sulfonyl, C₁-C₁₈alkylsulfonyloxy, phenylsulfonyloxy, (4-methylphenyl)sulfonyloxy, C₂-C₁₈alkanoyl, C₂-C₁₈alkanoyloxy, benzoyl and/or by benzoyloxy;
R₅ and R₆ independently of each other are hydrogen, C₃-C₃₀cycloalkyl, C₁-C₁₈alkyl, C₁-C₁₀haloalkyl, C₂-C₁₂alkenyl, C₄-C₃₀cycloalkenyl, phenyl-C₁-C₃-alkyl;
or independently of each other are C₂-C₁₈alkyl which is interrupted by one or more O, S, NR₇, O(CO), (CO)O, (CO)NR₇ and/or NR₇(CO);
or independently of each other are C₃-C₃₀cycloalkyl which is interrupted by one or more O, S, NR₇, O(CO), (CO)O, (CO)NR₇ and/or NR₇(CO);
or independently of each other are C₄-C₃₀cycloalkenyl which is interrupted by one or more O, S, NR₇, O(CO), (CO)O, (CO)NR₇, and/or NR₇(CO);
or R₅ and R₆ independently of each other are Ar, (CO)R₈, (CO)OR₄ and/or —SO₂R₈;
wherein R₅ and R₆ as C₃-C₃₀cycloalkyl, C₁-C₁₈alkyl, C₁-C₁₀haloalkyl, C₂-C₁₂alkenyl, O₄—C₃₀cycloalkenyl, phenyl-C₁-C₃-alkyl, interrupted C₂-C₁₈alkyl, interrupted C₃-C₃₀cycloalkyl, interrupted C₄-C₃₀cycloalkenyl and Ar optionally are substituted by one or more Ar, OH, C₁-C₁₈alkyl, C₁-C₁₀haloalkyl, phenyl-C₁-C₃-alkyl, C₃-C₃₀cycloalkyl, halogen, NO₂, CN, C₁-C₁₈alkoxy, phenoxy, phenoxycarbonyl, phenylthio, phenylthiocarbonyl, C₁-C₁₈dialkylamino, C₁-C₁₂alkylthio, C₂-C₁₈alkoxycarbonyl, C₂-C₁₀haloalkanoyl, halobenzoyl, C₁-C₁₈alkylsulfonyl, phenylsulfonyl, (4-methylphenyl)sulfonyl, C₁-C₁₈alkylsulfonyloxy, phenylsulfonyloxy, (4-methylphenyl)sulfonyloxy, C₂-C₁₈alkanoyl, C₂-C₁₈alkanoyloxy, benzoyl and/or by benzoyloxy;
or R₅ and R₆, together with the nitrogen atom to which they are attached, form a 5-, 6- or 7-membered ring which optionally is interrupted by one or more O, NR₇ or CO;
R₇ is hydrogen, C₃-C₃₀cycloalkyl, C₁-C₁₈alkyl, C₁-C₁₀haloalkyl, C₂-C₁₂alkenyl, O₄—C₃₀cycloalkenyl, phenyl-C₁-C₃-alkyl;
or is C₂-C₁₈alkyl which is interrupted by one or more O, S, O(CO) and/or (CO)O;
or is C₃-C₃₀cycloalkyl which is interrupted by one or more O, S, O(CO) and/or (CO)O;
or is C₄-C₃₀cycloalkenyl which is interrupted by one or more O, S, O(CO) and/or (CO)O; or R₇ is Ar, (CO)R₈, (CO)OR₄, (CO)NR₅R₆, and/or SO₂R₈;
wherein R₇ as C₃-C₃₀cycloalkyl, C₁-C₁₈alkyl, C₁-C₁₀haloalkyl, C₂-C₁₂alkenyl, C₄-C₃₀cycloalkenyl, phenyl-C₁-C₃-alkyl, interrupted C₂-C₁₈alkyl, interrupted C₃-C₃₀cycloalkyl, interrupted C₄-C₃₀cycloalkenyl and Ar optionally is substituted by one or more Ar, OH, C₁-C₁₈alkyl, C₁-C₁₀haloalkyl, phenyl-C₁-C₃-alkyl, C₃-C₃₀cycloalkyl, halogen, NO₂, CN, C₁-C₁₈alkoxy, phenoxy, phenoxycarbonyl, phenylthio, phenylthiocarbonyl, NR₅R₆, C₁-C₁₂alkylthio, C₂-C₁₈alkoxycarbonyl, C₂-C₁₀haloalkanoyl, halobenzoyl, C₁-C₁₈alkylsulfonyl, phenylsulfonyl, (4-methylphenyl)sulfonyl, C₁-C₁₈alkylsulfonyloxy, phenylsulfonyloxy, (4-methylphenyl)sulfonyloxy, C₂-C₁₈alkanoyl, C₂-C₁₈alkanoyloxy, benzoyl and/or by benzoyloxy;
R₈ is hydrogen, C₃-C₃₀cycloalkyl, C₁-C₁₀haloalkyl, C₂-C₁₂alkenyl, O₄—C₃₀cycloalkenyl, phenyl-C₁-C₃-alkyl, Ar, NR₅R₆;
or is C₂-C₁₈alkyl which is interrupted by one or more O, S, NR₇, O(CO), (CO)O, (CO)NR₇, and/or NR₇(CO);
or is C₃-C₃₀cycloalkyl which is interrupted by one or more O, S, NR₇, CO, O(CO), (CO)O, (CO)NR₇ and/or NR₇(CO);
or is C₄-C₃₀cycloalkenyl which is interrupted by one or more O, S, NR₇, O(CO), (CO)O, (CO)NR₇ and/or NR₇(CO);
wherein R₈ as C₃-C₃₀cycloalkyl, C₁-C₁₈alkyl, C₁-C₁₀haloalkyl, C₂-C₁₂alkenyl, C₄-C₃₀cycloalkenyl, phenyl-C₁-C₃-alkyl, Ar interrupted C₂-C₁₈alkyl, interrupted C₃-C₃₀cycloalkyl and interrupted C₄-C₃₀cycloalkenyl optionally is substituted by one or more Ar, OH, C₁-C₁₈alkyl, C₁-C₁₀haloalkyl, phenyl-C₁-C₃-alkyl, C₃-C₃₀cycloalkyl, halogen, NO₂, CN, C₁-C₁₈alkoxy, phenoxy, phenoxycarbonyl, phenylthio, phenylthiocarbonyl, NR₅R₆, C₁-C₁₂alkylthio, C₂-C₁₈alkoxycarbonyl, C₂-C₁₀haloalkanoyl, halobenzoyl, C₁-C₁₈alkylsulfonyl, phenylsulfonyl, (4-methylphenyl)sulfonyl, C₁-C₁₈alkylsulfonyloxy, phenylsulfonyloxy, (4-methylphenyl)sulfonyloxy, C₂-C₁₈alkanoyl, C₂-C₁₈alkanoyloxy, benzoyl and/or by benzoyloxy; and
Ar is phenyl, biphenyl, fluorenyl, naphthyl, anthracyl, phenanthryl, or heteroaryl, wherein the phenyl, biphenyl, fluorenyl, naphthyl, anthracyl, phenanthryl, or heteroaryl optionally are substituted by one or more C₃-C₃₀cycloalkyl, C₁-C₁₈alkyl, C₁-C₁₀haloalkyl, C₂-C₁₂alkenyl, C₄-C₃₀cycloalkenyl, phenyl-C₁-C₃-alkyl;
or are substituted by C₂-C₁₈alkyl which is interrupted by one or more O, S, NR₇, O(CO), (CO)O, (CO)NR₇ and/or NR₇(CO);
or are substituted by C₃-C₃₀cycloalkyl which is interrupted by one or more O, S, NR₇, O(CO), (CO)O, (CO)NR₇ and/or NR₇(CO);
or are substituted by C₄-C₃₀cycloalkenyl which is interrupted by one or more O, S, NR₇, O(CO), (CO)O, (CO)NR₇, and/or NR₇(CO);
or are substituted by halogen, NO₂, CN, phenyl, biphenyl, naphthyl, heteroaryl, (CO)R₈, (CO)OR₄, (CO)NR₅R₆, O(CO)R₈, O(CO)OR₄, O(CO)NR₅R₆, NR₇(CO)R₈, NR₇(CO)OR₄, OR₄, NR₅R₆, SR₇, SOR₈, SO₂R₈ and/or OSO₂R₈, optionally the substituents C₁-C₁₈alkyl, C₂-C₁₂alkenyl, (CO)R₈, (CO)OR₄, (CO)NR₅R₆, O(CO)R₈, O(CO)OR₄, O(CO)NR₅R₆, NR₇(CO)R₈, NR₇(CO)OR₄, OR₄, NR₅R₆, SR₇, SOR₈, SO₂R₈ and/or OSO₂R₈, form 5-, 6- or 7-membered rings, via the radicals C₁-C₁₈alkyl, C₂-C₁₂alkenyl, R₄, R₅, R₆, R₇ and/or R₈, with further substituents on the phenyl, biphenyl, fluorenyl, naphthyl, anthracyl, phenanthryl or heteroaryl or with one of the carbon atoms of phenyl, biphenyl, fluorenyl, naphthyl, anthracyl, phenanthryl, or heteroaryl.

4. A chemically amplified photoresist composition comprising
(a) a compound which cures upon the action of an acid or a compound whose solubility is increased upon the action of an acid; and/or
(b) as photosensitive acid donor and optionally as compound whose solubility is increased upon the action of an acid, at least one compound of the formula I according to claim 1; and/or a polymer comprising at least one repeating unit derived from the compound of the formula I according to claim 1 and optionally repeating units derived from ethylenically unsaturated compounds of formula II according to claim 3.

5. A chemically amplified photoresist composition according to claim 4, which is a positive resist.

6. A chemically amplified photoresist composition according to claim 4, which is a negative resist.

7. A chemically amplified photoresist composition according to claim 4, in addition to components (b) or components (a) and (b), comprising further additives (c), further photosensitive acid donor compounds (b1), further photoinitiators (d), and/or sensitizers (e).

8. A process for the preparation of a photoresist by
(1) applying to a substrate a composition according to claim 4;
(2) post apply baking the composition at temperatures between 60° C. and 160° C.;
(3) image-wise irradiating with light of wavelengths between 10 nm and 1500 nm;
(4) optionally post exposure baking the composition at temperatures between 60° C. and 160° C.; and
(5) developing with a solvent or with an aqueous alkaline developer.

9. A composition comprising
(b) as photosensitive acid donor and as compound whose solubility is increased upon the action of an acid, at least one compound of the formula I according to claim 1; and/or a polymer comprising at least one repeating unit derived from the compound of the formula I according to claim 1 and repeating units derived from ethylenically unsaturated compounds of formula II according to claim 3.

10. A composition comprising
(a) a compound which cures upon the action of an acid or a compound whose solubility is increased upon the action of an acid; and
(b) as photosensitive acid donor and optionally as compound whose solubility is increased upon the action of an acid, at least one compound of the formula I according to claim 1; and/or a polymer comprising at least one repeating unit derived from the compound of the formula I according to claim 1 and optionally repeating units derived from ethylenically unsaturated compounds of formula II according to claim 3.

11. A method of using the compounds of the formula I according to claim 1; or polymers comprising at least one repeating unit derived from the compound of the formula I according to claim 1 and optionally repeating units derived from ethylenically unsaturated compounds of formula II according to claim 3 as photosensitive acid donors in compositions that can be crosslinked under the action of an acid and/or as dissolution enhancers in compositions wherein the solubility is increased under the action of an acid.

12. A method of using the compounds of the formula I according to claim 1; or polymers comprising at least one repeating unit derived from the compound of the formula I according to claim 1 and optionally repeating units derived from ethylenically unsaturated compounds of formula II according to claim 3, as photosensitive acid donors in the preparation of surface coatings, printing inks, printing plates, dental compositions, colour filters, resists or image-recording materials, or image-recording materials for recording holographic images.

13. A method of using the compounds of the formula I according to claim 1; or polymers comprising at least one repeating unit derived from the compound of the formula I according to claim 1 and optionally repeating units derived from ethylenically unsaturated compounds selected from the of formula II according to claim 3, as photosensitive acid donors in the preparation of colour filters or chemically amplified resists.

14. A color filter prepared by providing red, green and blue picture elements and a black matrix, all comprising a photosensitive resin and a pigment and/or dye on a transparent substrate and providing a transparent electrode either on the surface of the substrate or on the surface of the color filter layer, wherein said photosensitive resin comprises polymers comprising at least one repeating unit derived from the compound of the formula I according to claim 1 and optionally repeating units derived from ethylenically unsaturated compounds of formula II according to claim 3, as photosensitive acid donors.

15. A chemically amplified photoresist composition according to claim 5, in addition to components (b) or components (a) and (b), comprising further additives (c), further photosensitive acid donor compounds (b1), further photoinitiators (d), and/or sensitizers (e).

16. A chemically amplified photoresist composition according to claim 6, in addition to components (b) or components (a) and (b), comprising further additives (c), further photosensitive acid donor compounds (b1), further photoinitiators (d), and/or sensitizers (e).

17. A compound of formula I according to claim 1, wherein the compound is select from (a)-(o) of the following: 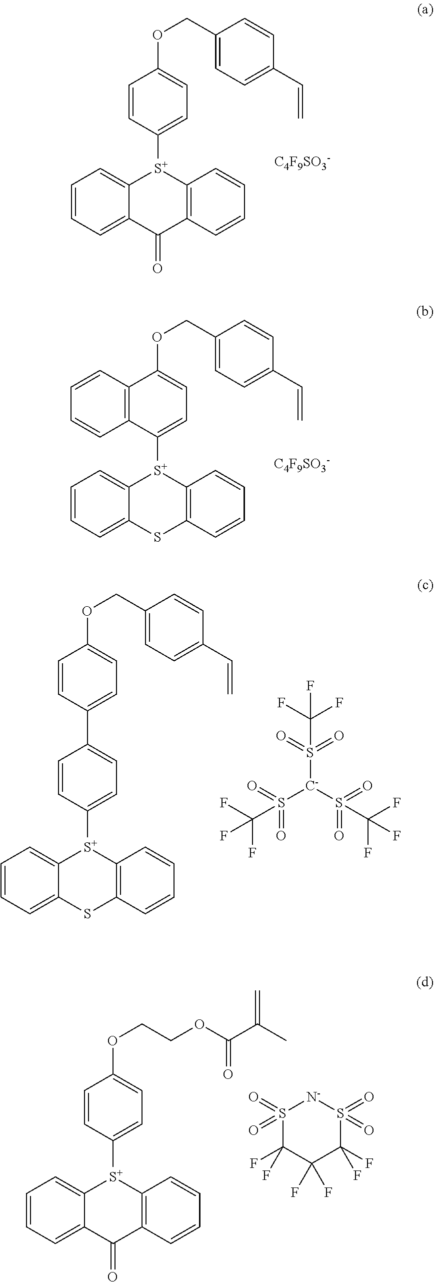 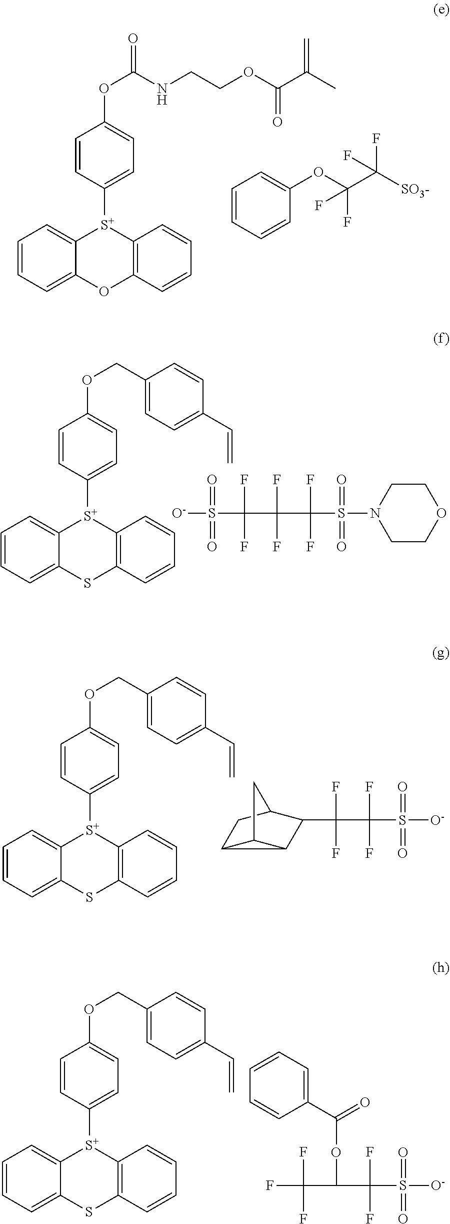 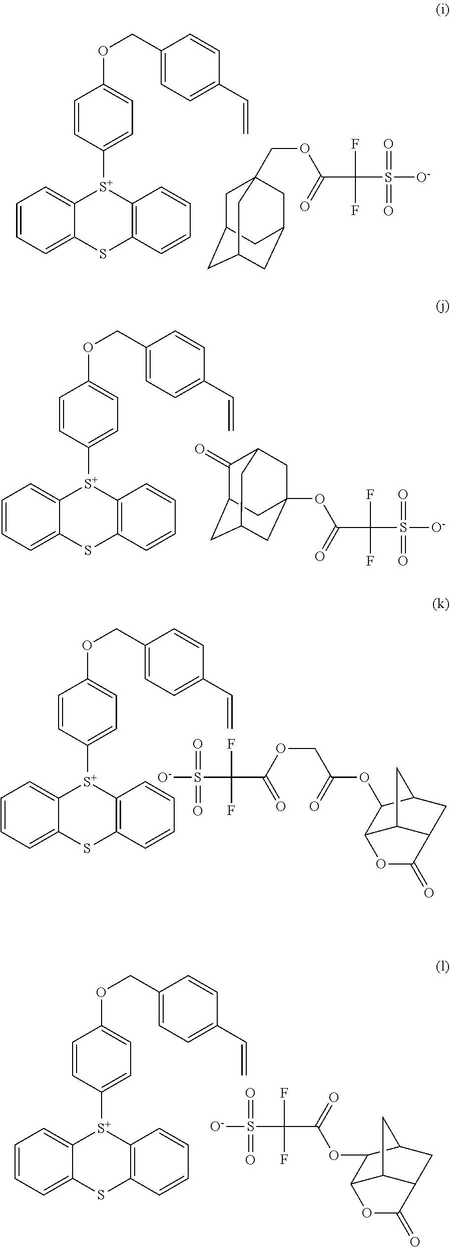 [chemical structure]
